(19) United States
(12) Patent Application Publication
(10) Pub. No.: US 2016/0058751 A1
(43) Pub. Date: Mar. 3, 2016

(54) COMPOSITION AND METHOD FOR TREATING CANCER

(71) Applicant: CELLWORKS GROUP, INC., Saratoga, CA (US)

(72) Inventors: Shireen VALI, Irvine, CA (US); Shahabuddin Usmani, Bangalore, Karnataka (IN); Zeba Sultanan, Bangalore, Karnataka (IN); Shweta Kapoor, Bangalore, Karnataka (IN); Ansu Kumar, Bangalore, Karnataka (IN); Ashish Kumar Agrawal, Bangalore, Karnataka (IN)

(73) Assignee: CELLWORKS GROUP, INC., Saratoga, CA (US)

(21) Appl. No.: 14/780,724

(22) PCT Filed: Mar. 25, 2014
(86) PCT No.: PCT/US14/31769

§ 371 Date: Sep. 28, 2015

(30) Foreign Application Priority Data
Mar. 28, 2013 (IN) 1422/CHE/2013

(51) Int. Cl. A61K 31/4745; A61K 31/4709; A61K 31/18; A61K 31/4545; A61K 31/44; A61K 31/40

(52) U.S. Cl. CPC A61K 31/4745; A61K 31/44; A61K 31/40; A61K 31/18; A61K 31/4545; A61K 31/4709

(57) ABSTRACT

The present disclosure relates to a pharmaceutical composition and a kit to treat cancer. The disclosure provides a combination of compounds for use in the treatment of cancer. The disclosure further provides a process of preparing the composition and a method of treating a cancer associated with a RAS mutation or a RAS mutation along with any other mutation.

### CROSS REFERENCE

This application claims the benefit of Indian Provisional Application No. 1422/CHE/2013, filed on Mar. 28, 2013, the contents of which is incorporated by reference in its entirety.

### BACKGROUND

Cancer, an uncontrolled proliferation of cells, is a multifactorial disease characterized by tumor formation, growth, and in some instances, metastasis. Current cancer therapies vary depending upon the localization and stage of the cancer but generally include a combination of surgery, systemic therapy, radiation therapy, and chemotherapy. Despite the effort that has been devoted to the development of anti-cancer strategies, many of them remain unefficacious for specific cancers. Many anti-cancer agents exist, but many cancers remain difficult to remedy.

### SUMMARY OF THE INVENTION

In some embodiments, the invention provides a pharmaceutical composition comprising: a) i) a kinsase inhibitor or a pharmaceutically-acceptable salt thereof; and ii) an inhibitor of an enzyme that is involved in lipid metabolism or a pharmaceutically-acceptable salt thereof; and b) a pharmaceutically-acceptable excipient, wherein the composition is a unit dosage form.

In some embodiments, the invention provides a kit comprising: a) i) a kinsase inhibitor or a pharmaceutically-acceptable salt thereof; and ii) an inhibitor of an enzyme that is involved in lipid metabolism or a pharmaceutically-acceptable salt thereof; and b) written instructions on use of the kit for treatment of a condition.

In some embodiments, the invention provides a method for treating a condition in a subject in need thereof, the method comprising administering to the subject: i) a therapeutically-effective amount of a kinsase inhibitor or a pharmaceutically-acceptable salt thereof; and ii) a therapeutically-effective amount of an inhibitor of an enzyme that is involved in lipid metabolism or a pharmaceutically-acceptable salt thereof.

### BRIEF DESCRIPTION OF THE FIGURES

FIG. 1 depicts the effect of the combination of CW137 and CW231a on a non-triggered normal epithelial cell.

FIG. 2 depicts the effect of the combination CW137 and CW231b on a non-triggered normal epithelial cell.

FIG. 3 depicts the effect of the combination of CW147 and CW231a on a non-triggered normal epithelial cell.

FIG. 4 depicts the effect of the combination CW147 and CW231b on a non-triggered normal epithelial cell.

FIG. 5 depicts the effect of CW137 alone, CW231a alone, or a combination of CW137 and CW231a on the viability of RAS and non-RAS cell lines. Cell lines and the respective profiles include A549 (KRAS profile), HCT116 (KRAS and PIK3CA profile), H1299 (NRAS and PTEN profile), IM9 (NRAS profile), H1650 (EGFR and PTEN profile), SW48 (EGFR profile), CaCo2 (CTNNB1 and P53 profile) and SKMM2 (CDKN2C and P53 profile). In the two drug combinations, each drug was administered at an IC₂₅ concentration, i.e. a concentration that causes a 25% reduction in viability calculated based on viability change in HCT116 profile.

FIG. 6 depicts the effect of CW137 alone, CW231a alone, or a combination of CW137 and CW231a on the proliferation of RAS and non-RAS cell lines. In the two drug combinations, each drug was administered at an IC₂₅ concentration.

FIG. 7 depicts the effect of CW137 alone, CW231b alone, or a combination of CW137 and CW231b on the viability of RAS and non-RAS cell lines. In the two drug combinations, each drug was administered at an IC₂₅ concentration.

FIG. 8 depicts the effect of CW137 alone, CW231b alone, or a combination of CW137 and CW231b on the proliferation of RAS and non-RAS cell lines. In the two drug combinations, each drug was administered at an IC₂₅ concentration.

FIG. 9 depicts the effect of CW147 alone, CW231a alone, or a combination of CW147 and CW231a on the viability of RAS and non-RAS cell lines. In the two drug combinations, each drug was administered at an IC₂₅ concentration.

FIG. 10 depicts the effect of CW147 alone, CW231a alone, or a combination of CW147 and CW231a on the proliferation of RAS and non-RAS cell lines. In the two drug combinations, each drug was administered at an IC₂₅ concentration.

FIG. 11 depicts the effect of CW147 alone, CW231b alone, or a combination of CW147 and CW231b on the viability of RAS and non-RAS cell lines. In the two drug combinations, each drug was administered at an IC₂₅ concentration.

FIG. 12 depicts the effect of CW147 alone, CW231b alone, or a combination of CW147 and CW231b on the proliferation of RAS and non-RAS cell lines. In the two drug combinations, each drug was administered at an IC₂₅ concentration.

FIG. 13 depicts the effect of CW137 alone, CW231a alone, or a combination of CW137 and CW231a on the viability of the multiple myeloma cell lines IM9 and SKMM2. In the two drug combinations, each drug was administered at an IC₂₅ concentration.

FIG. 14 depicts the effect of CW137 alone, CW231a alone, or a combination of CW137 and CW231a on the proliferation of the multiple myeloma cell lines IM9 and SKMM2. In the two drug combinations, each drug was administered at an IC₂₅ concentration.

FIG. 15 depicts the effect of CW137 alone, CW231b alone, or a combination of CW137 and CW231b on the viability of the multiple myeloma cell lines IM9 and SKMM2. In the two drug combinations, each drug was administered at an IC₂₅ concentration.

FIG. 16 depicts the effect of CW137 alone, CW231b alone, or a combination of CW137 and CW231b on the proliferation of the multiple myeloma cell lines IM9 and SKMM2. In the two drug combinations, each drug was administered at an IC₂₅ concentration.

FIG. 17 depicts the effect of CW147 alone, CW231a alone, or a combination of CW147 and CW231a on the viability of the multiple myeloma cell lines IM9 and SKMM2. In the two drug combinations, each drug was administered at an IC₂₅ concentration.

FIG. 18 depicts the effect of CW147 alone, CW231a alone, or a combination of CW147 and CW231a on the proliferation of the multiple myeloma cell lines IM9 and SKMM2. In the two drug combinations, each drug was administered at an IC₂₅ concentration.

FIG. 19 depicts the effect of CW147 alone, CW231b alone, or a combination of CW147 and CW231b on the viability of the multiple myeloma cell lines IM9 and SKMM2. In the two drug combinations, each drug was administered at an IC₂₅ concentration.

FIG. 20 depicts the effect of CW147 alone, CW231b alone, or a combination of CW147 and CW231b on the proliferation of the multiple myeloma cell lines IM9 and SKMM2. In the two drug combinations, each drug was administered at an IC₂₅ concentration.

FIG. 21 depicts the effect of CW137 alone, CW231a alone, or a combination of CW137 and CW231a on BAX levels in RAS and non-RAS cell lines. BAX is a key biomarker for apoptosis. Cell lines and the respective profiles include A549 (KRAS profile), HCT116 (KRAS and PIK3CA profile), H1299 (NRAS and PTEN profile), IM9 (NRAS profile), H1650 (EGFR and PTEN profile), SW48 (EGFR profile), CaCo2 (CTNNB1 and P53 profile) and SKMM2 (CDKN2C and p53 profile). In the two drug combinations, each drug was administered at an IC₂₅ concentration.

FIG. 22 depicts the effect of CW137 alone, CW231a alone, or a combination of CW137 and CW231a on Casp9 levels in RAS and non-RAS cell lines. Casp9 is a key biomarker for apoptosis. In the two drug combinations, each drug was administered at an IC₂₅ concentration.

FIG. 23 depicts the effect of CW137 alone, CW231a alone, or a combination of CW137 and CW231a on CDK4-CCNDI complex levels in RAS and non-RAS cell lines. CDK4-CCND1 is a key biomarker for proliferation. In the two drug combinations, each drug was administered at an IC₂₅ concentration.

FIG. 24 depicts the effect of CW137 alone, CW231a alone, or a combination of CW137 and CW231a on CDC2-CCNB1 complex levels in RAS and non-RAS cell lines. CDC2-CCNB1 is a key biomarker for proliferation. In the two drug combinations, each drug was administered at an IC₂₅ concentration.

FIG. 25 depicts the effect of CW137 alone, CW231a alone, or a combination of CW137 and CW231a on PTTG1 levels in RAS and non-RAS cell lines. PTTG1 is a key biomarker for protein translation and cell growth. In the two drug combinations, each drug was administered at an IC₂₅ concentration.

FIG. 26 depicts the effect of CW137 alone, CW231a alone, or a combination of CW137 and CW231a on MYC-MAX complex levels in RAS and non-RAS cell lines. The MYC-MAX complex is a key biomarker for cell growth. In the two drug combinations, each drug was administered at an IC₂₅ concentration.

FIG. 27 depicts the effect of CW137 alone, CW231a alone, or a combination of CW137 and CW231a on p-AKT levels in RAS and non-RAS cell lines. p-AKT is a key biomarker for cell survival. In the two drug combinations, each drug was administered at an IC₂₅ concentration.

FIG. 28 depicts the effect of CW137 alone, CW231a alone, or a combination of CW137 and CW231a on BIRC2 levels in RAS and non-RAS cell lines. BIRC2 is a key biomarker for cell survival. In the two drug combinations, each drug was administered at an IC₂₅ concentration.

FIG. 29 depicts the effect of CW137 alone, CW231b alone, or a combination of CW137 and CW231b on BAX levels in RAS and non-RAS cell lines. In the two drug combinations, each drug was administered at an IC₂₅ concentration.

FIG. 30 depicts the effect of CW137 alone, CW231b alone, or a combination of CW137 and CW231b on Casp9 levels in RAS and non-RAS cell lines. In the two drug combinations, each drug was administered at an IC₂₅ concentration.

FIG. 31 depicts the effect of CW137 alone, CW231b alone, or a combination of CW137 and CW231b on CDK4-CCND1 complex levels in RAS and non-RAS cell lines. In the two drug combinations, each drug was administered at an IC₂₅ concentration.

FIG. 32 depicts the effect of CW137 alone, CW231b alone, or a combination CW137 and CW231b on CDC2-CCNB1 complex levels in RAS and non-RAS cell lines. In the two drug combinations, each drug was administered at an IC₂₅ concentration.

FIG. 33 depicts the effect of CW137 alone, CW231b alone, or a combination of CW137 and CW231b on PTTG1 levels in RAS and non-RAS cell lines. In the two drug combinations, each drug was administered at an IC₂₅ concentration.

FIG. 34 depicts the effect of CW137 alone, CW231b alone, or a combination of CW137 and CW231b on MYC-MAX complex levels in RAS and non-RAS cell lines. In the two drug combinations, each drug was administered at an IC₂₅ concentration.

FIG. 35 depicts the effect of CW137 alone, CW231b alone, or a combination of CW137 and CW231b on p-AKT levels in RAS and non-RAS cell lines. In the two drug combinations, each drug was administered at an IC₂₅ concentration.

FIG. 36 depicts the effect of CW137 alone, CW231b alone, or a combination of CW137 and CW231b on BIRC2 levels in RAS and non-RAS cell lines. In the two drug combinations, each drug was administered at an IC₂₅ concentration.

FIG. 37 depicts the effect of CW147 alone, CW231a alone, or a combination of CW147 and CW231a on BAX levels in RAS and non-RAS cell lines. In the two drug combinations, each drug was administered at an IC₂₅ concentration.

FIG. 38 depicts the effect of CW147 alone, CW231a alone, or a combination of CW147 and CW231a on CASP9 levels in RAS and non-RAS cell lines. In the two drug combinations, each drug was administered at an IC₂₅ concentration.

FIG. 39 depicts the effect of CW147 alone, CW231a alone, or a combination of CW147 and CW231a on CDK4-CCND1 complex levels in RAS and non-RAS cell lines. In the two drug combinations, each drug was administered at an IC₂₅ concentration.

FIG. 40 depicts the effect of CW147 alone, CW231a alone, or a combination of CW147 and CW231a on CDC2-CCNB1 complex levels in RAS and non-RAS cell lines. In the two drug combinations, each drug was administered at an IC₂₅ concentration.

FIG. 41 depicts the effect of CW147 alone, CW231a alone, or a combination of CW147 and CW231a on PTTG1 levels in RAS and non-RAS cell lines. In the two drug combinations, each drug was administered at an IC₂₅ concentration.

FIG. 42 depicts the effect of CW147 alone, CW231a alone, or a combination of CW147 and CW231a on MYC-MAX complex levels in RAS and non-RAS cell lines. In the two drug combinations, each drug was administered at an IC₂₅ concentration.

FIG. 43 depicts the effect of CW147 alone, CW231a alone, or a combination of CW147 and CW231a on p-AKT levels in RAS and non-RAS cell lines. In the two drug combinations, each drug was administered at an IC₂₅ concentration.

FIG. 44 depicts the effect of CW147 alone, CW231a alone, or a combination of CW147 and CW231a on BIRC2 levels in RAS and non-RAS cell lines. In the two drug combinations, each drug was administered at an IC₂₅ concentration.

FIG. 45 depicts the effect of CW147 alone, CW231b alone, or a combination of CW147 and CW231b on BAX levels in RAS and non-RAS cell lines. In the two drug combinations, each drug was administered at an IC₂₅ concentration.

FIG. 46 depicts the effect of CW147 alone, CW231b alone, or a combination of CW147 and CW231b on CASP9 levels in RAS and non-RAS cell lines. In the two drug combinations, each drug was administered at an IC₂₅ concentration.

FIG. 47 depicts the effect of CW147 alone, CW231b alone, or a combination of CW147 and CW231b on CDK4-CCND1 complex levels in RAS and non-RAS cell lines. In the two drug combinations, each drug was administered at an IC₂₅ concentration.

FIG. 48 depicts the effect of CW147 alone, CW231b alone, or a combination of CW147 and CW231b on CDC2-CCNB1 complex levels in RAS and non-RAS cell lines. In the two drug combinations, each drug was administered at an IC₂₅ concentration.

FIG. 49 depicts the effect of CW147 alone, CW231b alone, or a combination of CW147 and CW231b on PTTG1 levels in RAS and non-RAS cell lines. In the two drug combinations, each drug was administered at an IC₂₅ concentration.

FIG. 50 depicts the effect of CW147 alone, CW231b alone, or a combination of CW147 and CW231b on MYC-MAX complex levels in RAS and non-RAS cell lines. In the two drug combinations, each drug was administered at an IC₂₅ concentration.

FIG. 51 depicts the effect of CW147 alone, CW231b alone, or a combination of CW147 and CW231b on p-AKT levels in RAS and non-RAS cell lines. In the two drug combinations, each drug was administered at an IC₂₅ concentration.

FIG. 52 depicts the effect of CW147 alone, CW231b alone, or a combination of CW147 and CW231b on BIRC2 levels in RAS and non-RAS cell lines. In the two drug combinations, each drug was administered at an IC₂₅ concentration.

FIG. 53 depicts the scientific rationale for the effect of different combinations of CW137 and CW231a on the key phenotypes of RAS driven cancer.

FIG. 54 depicts the scientific rationale for the effect of different combinations of CW137 and CW231b on the key phenotypes of RAS driven cancer.

FIG. 55 depicts the scientific rationale for the effect of different combinations of CW147 and CW231a on the key phenotypes of RAS driven cancer.

FIG. 56 depicts the scientific rationale for the effect of different combinations of CW147 and CW231b on the key phenotypes of RAS driven cancer.

FIG. 57 depicts the effect of a combination of CW137 and CW231a versus Erlotinib on the viability of three KRAS mutated cell lines—A549, HCT116 and H1299.

FIG. 58 depicts the effect of a combination of CW137 and CW231a versus Erlotinib on the proliferation of three KRAS mutated cell lines—A549, HCT116 and H1299.

FIG. 59 depicts the effect of a combination of CW137 and CW231b versus Erlotinib on the viability of three KRAS mutated cell lines—A549, HCT116 and H1299.

FIG. 60 depicts the effect of a combination of CW137 and CW231b versus Erlotinib on the proliferation of three KRAS mutated cell lines—A549, HCT116 and H1299.

FIG. 61 depicts the effect of a combination of CW147 and CW231a versus Erlotinib on the viability of three KRAS mutated cell lines—A549, HCT116 and H1299.

FIG. 62 depicts the effect of a combination of CW147 and CW231a versus Erlotinib on the proliferation of three KRAS mutated cell lines—A549, HCT116 and H1299.

FIG. 63 depicts the effect of a combination of CW147 and CW231b versus Erlotinib on the viability of three KRAS mutated cell lines—A549, HCT116 and H1299.

FIG. 64 depicts the effect of a combination of CW147 and CW231b versus Erlotinib on the proliferation of three KRAS mutated cell lines—A549, HCT116 and H1299.

FIG. 65 depicts the dose response of single drug CW137 on the viability of KRAS and non-KRAS cell lines.

FIG. 66 depicts the dose response of single drug CW147 on the viability of KRAS and non-KRAS cell lines.

FIG. 67 depicts the dose response of single drug CW231a on the viability of KRAS and non-KRAS cell lines.

FIG. 68 depicts the dose response of single drug CW231b on the viability of KRAS and non-KRAS cell lines.

FIG. 69 depicts the effect of a low concentration of the RAS inhibitor CW137 on the KRAS mutant cell line A549.

FIG. 70 depicts the effect of a low concentration of the RAS inhibitor CW137 on the KRAS wild-type cell line H1650.

FIG. 71 depicts the percentage change in the relative growth of the KRAS cell line A549 using single and two drug combinations of CW137 and CW231a. On the x-axis is the increasing dose of CW231a tested individually and with two fixed doses of CW137 at 0.5 μM and 5 μM.

FIG. 72 depicts the percentage change in the relative growth of the non-KRAS cell line H1650 using single and two drug combinations of CW137 and CW231a. On the x-axis is the increasing dose of CW231a tested individually and in combination with two fixed doses of CW137 at 0.5 μM and 5 μM.

FIG. 73 depicts the percentage change in the relative growth of the KRAS cell line HCT116 using single and two drug combinations of CW137 and CW231a. On the x-axis is the increasing dose of CW231a tested individually and in combination with two fixed doses of CW137 at 0.5 μm and 5 μM.

FIG. 74 depicts the percentage change in the relative growth of the non-KRAS cell line CaCo2 using single and two drug combinations of CW137 and CW231a. On the x-axis is the increasing dose of CW231a tested individually and in combination with two fixed doses of CW137 at 0.5 μM and 5 μM.

FIG. 75 depicts the percentage change in the relative growth of the non-KRAS cell line SW48 using single and two drug combinations of CW137 and CW231a. On the x-axis is the increasing dose of CW231a tested individually and in combination with two fixed doses of CW137 at 0.5 μM and 5 μM.

FIG. 76 depicts the percentage change in the relative growth of the cell lines harboring KRAS mutations and cell lines with wild type KRAS upon administration of single and two drug combinations of CW137 and CW231a at a concentration of 5 μM and 10 μM, respectively.

FIG. 77 depicts the percentage change in the relative growth of the cell lines harboring KRAS mutations and cell lines with wild type KRAS upon administration of single and two drug combination of CW137 and CW231a at a concentration of 5 μM and 40 μM, respectively.

FIG. 78 depicts the percentage change in the relative growth of the KRAS cell line A549 using single and two drug combinations of CW137 and CW231b. On the x-axis is the increasing dose of CW231b tested individually and in combination with two fixed doses of CW137 at 3 μM and 5 μM.

FIG. 79 depicts the percentage change in the relative growth of the non-KRAS cell line H1650 using single and two drug combinations of CW137 and CW231b. On the x-axis is the increasing dose of CW231b tested individually and in combination with two fixed doses of CW137 at 3 μM and 5 μM.

FIG. 80 depicts the percentage change in the relative growth of the KRAS cell line HCT116 using single and two drug combinations of CW137 and CW231b. On the x-axis is the increasing dose of CW231b tested individually and in combination with two fixed doses of CW137 at 3 μM and 5 μM.

FIG. 81 depicts the percentage change in the relative growth of the non-KRAS cell line SW48 using single and two drug combinations of CW137 and CW231b. On the x-axis is the increasing dose of CW231b tested individually and in combination with two fixed doses of CW137 at 3 μM and 5 μM.

FIG. 82 depicts the percentage change in the relative growth of the cell lines harboring a KRAS mutation and cell lines with wild type KRAS upon administration of single and two drug combinations of CW137 and CW231b at a concentration of 3 μM and 20 μM, respectively

FIG. 83 depicts the percentage change in the relative growth of the cell lines harboring KRAS mutation and cell lines with wild type KRAS upon administration of single and two drug combination of CW137 and CW231b at a concentration of 5 μM and 20 μM, respectively.

FIG. 84 depicts the percentage change in the relative growth of the KRAS cell line A549 using single and two drug combinations of CW147 and CW231a. On the x-axis is the increasing dose of CW231a tested individually and in combination with two fixed doses of CW147 at 1 μM and 3 μM.

FIG. 85 depicts the percentage change in the relative growth of the non-KRAS cell line H1650 using single and two drug combinations of CW147 and CW231a. On the x-axis is the increasing dose of CW231a tested individually and in combination with two fixed doses of CW147 at 1 μM and 3 μM.

FIG. 86 depicts the percentage change in the relative growth of the KRAS cell line HCT116 using single and two drug combinations of CW147 and CW231a. On the x-axis is the increasing dose of CW231a tested individually and in combination with two fixed doses of CW147 at 1 μM and 3 μM.

FIG. 87 depicts the percentage change in the relative growth of the non-KRAS cell line SW48 using single and two drug combinations of CW147 and CW231a. On the x-axis is the increasing dose of CW231a tested individually and in combination with two fixed doses of CW147 at 1 μM and 3 μM.

FIG. 88 depicts the percentage change in the relative growth of the cell lines harboring KRAS mutation and cell lines with wild type KRAS upon administration of single and two drug combination of CW147 and CW231a at a concentration of 1 μM and 40 μM, respectively.

FIG. 89 depicts the percentage change in the relative growth of the cell lines harboring KRAS mutation and cell lines with wild type KRAS upon administration of single and two drug combination of CW147 and CW231a at a concentration of 3 μM and 40 μM, respectively.

FIG. 90 depicts the percentage change in the relative growth of the KRAS cell line A549 using single and two drug combinations of CW147 and CW231b. On the x-axis is the increasing dose of CW231b tested individually and in combination with two fixed doses of CW147 at 1 μM and 3 μM.

FIG. 91 depicts the percentage change in the relative growth of the non-KRAS cell line H1650 using single and two drug combinations of CW147 and CW231b. On the x-axis is the increasing dose of CW231b tested individually and in combination with two fixed doses of CW147 at 1 μM and 3 μM.

FIG. 92 depicts the percentage change in the relative growth of the KRAS cell line HCT116 using single and two drug combinations of CW147 and CW231a. On the x-axis is the increasing concentration of CW231a tested individually (reference made to DMSO), and in combination with fixed doses of CW147 at GI₁₅, GI₃₀ and GI₅₀ concentrations, respectively.

FIG. 93 depicts the zoomed in plot of FIG. 29.1 between the concentrations 1 to 31.6 μM of CW231a.

FIG. 94 depicts the percentage change in the relative growth of the KRAS cell line HCT116 upon administration of single and two drug combinations of CW147 and CW231a, wherein CW147 was at a concentration GI₁₅, GI₃₀ and GI₅₀, respectively, and CW231a was at a concentration of 10 μM.

FIG. 95 depicts the percentage change in the relative growth of the KRAS cell line CAPAN1 using single and two drug combinations of CW147 and CW231a. On the x-axis is the increasing concentration of CW231a tested individually (reference made to DMSO), and in combination with fixed doses of CW147 at GI15, GI30 and GI50 concentrations, respectively.

FIG. 96 depicts the zoomed in plot of FIG. 30.1 between the concentrations 1 to 31.6 μM of CW231a.

FIG. 97 depicts the percentage change in the relative growth of the KRAS cell line CAPAN1 upon administration of single and two drug combinations of CW147 and CW231a, wherein the concentration of CW147 was of GI₁₅, GI₃₀, GI₅₀, respectively and the concentration of CW231a was 10 μM.

FIG. 98 depicts the percentage change in the relative growth of the cell line SW480 using single and two drug combinations of CW147 and CW231a. On the x-axis is the increasing concentration of CW231a tested individually (reference made to DMSO), and in combination with fixed doses of CW147 at GI₁₅, GI₃₀ and GI₅₀ concentrations, respectively.

FIG. 99 depicts the zoomed in plot of FIG. 98 between the concentrations 1 to 31.6 μM of CW231a.

FIG. 100 depicts the percentage change in the relative growth of the SW480 cell lines upon administration of single and two drug combinations of CW147 and CW231a, wherein the concentration of CW147 was GI₁₅, GI₃₀, GI₅₀, respectively, and the concentration of CW231a was 10 μM.

FIG. 101 depicts the percentage change in the relative growth of the cell line DLD1 using single and two drug combinations of CW147 and CW231a. On the x-axis is the increasing concentrations of CW231a tested individually (reference made to DMSO), and in combination with fixed doses of CW147 at GI₁₅, GI₃₀ and GI₅₀ concentrations, respectively.

FIG. 102 depicts the zoomed in plot of FIG. 32.1 between the concentrations 1 to 31.6 μM of CW231a.

FIG. 103 depicts the percentage change in the relative growth of the DLD1 cell lines upon administration of single and two drug combinations of CW147 and CW231a, wherein the concentration of CW147 was at concentrations of GI₁₅, GI₃₀, GI₅₀, respectively, and concentration of CW231a was 10 μM.

FIG. 104 depicts the percentage change in the relative growth of the KRAS cell line HCT116 using single and two drug combinations of CW147 and CW231a. On the x-axis is the increasing dose of CW231a tested individually and in combination with fixed doses of CW147 at 4 μM, 8 μM and 12 μM, respectively.

FIG. 105 depicts the percentage change in the relative growth of the KRAS cell line HCT116 upon administration of single and two drug combinations of CW231a and CW147, wherein the concentration of CW231a was 12.5 μM, 25 μM and 50 μM and the concentration of CW147 was 8 μM.

FIG. 106 depicts the percentage change in the relative growth of the KRAS cell line HCT116 using single and two drug combinations of CW147 and CW231c. On the x-axis is the increasing concentration of CW231c tested individually and in combination with fixed doses of CW147 at 4 μM, 8 μM and 12 μM, respectively.

FIG. 107 depicts the percentage change in the relative growth of the KRAS cell line HCT116 upon administration of single and two drug combinations of CW231c and CW147, wherein the concentration of CW231c was 0.5 μM, 1 μM, 3 μM and the concentration of CW147 was 8 μM.

FIG. 108 depicts the effect of CW137 alone (Cohort SFB) and in combinations of CW137 and CW231a (Cohort AT & SFB) versus untreated on the tumor growth of the KRAS colorectal cancer profile HCT116 xenograft.

FIG. 109 depicts a Kaplan-Meier survival curve for the KRAS colorectal cancer profile HCT116 xenograft study for the effect of CW137 alone (cohort SFB) and a combination of CW137 with CW231a (cohort SFB-AT) versus untreated mice.

FIG. 110 depicts the effect of CW137 alone (cohort SFB) and a combination of CW137 with CW231a (cohort AT & SFB) versus untreated mice on the body weight of the KRAS colorectal cancer profile HCT116 xenograft, wherein the body weight plot indicates that the two drug combination was similar to the untreated and the drug combination did not have any unexpected toxicity effects that would relate to loss of body weight.

FIG. 111 depicts the effect of a combination of CW137 and CW231a (Cohort AT & SFB) versus untreated mice on the tumor growth of the KRAS pancreatic cancer profile CAPAN1 xenograft.

FIG. 112 depicts a Kaplan-Meier survival curve for the KRAS pancreatic cancer profile CAPAN1 xenograft due to the effect of CW137 alone (cohort SFB) and combination of CW137 and CW231a (cohort SFB-AT) versus untreated animals.

FIG. 113 depicts the effect of CW137 alone (cohort SFB) and combination of CW137 and CW231a (cohort AT & SFB) versus untreated animals on the body weight of the KRAS pancreatic cancer profile CAPAN1 xenograft.

### DETAILED DESCRIPTION

Cancer is a group of more than 100 diseases that develops over time and involves uncontrolled proliferation of cells. Although cancer can develop in virtually any of the body's tissues, and each type of cancer has unique features, the basic processes that cause cancer can be similar in all forms of the disease. Dis-regulation of multiple signaling pathways and loss of cross-talk between them can result in cancer.

Cancer can begin when a cell breaks free from the normal restraints on cell division and begins abnormal proliferation. Genetic mutations in the cell can preclude the ability of the cell to repair damaged DNA or execute apoptosis. These events, in combination with a host of other factors, can result in uncontrolled division and growth of cells. Genetic mutations can also affect oncogenes and tumor suppressors, potentially activating or suppressing their activating in an inappropriate manner, also leading to uncontrolled cell division.

All of the cells produced by division of this first, ancestral cell and its progeny can also display inappropriate proliferation. A tumor, or mass of cells, formed from these abnormal cells can remain within the original tissue (in situ cancer), or can invade nearby tissues (invasive cancer). An invasive tumor is said to be malignant, and cells shed into the blood or lymph from a malignant tumor can establish new tumors (metastases) throughout the body. Tumors threaten an individual's life when their growth disrupts the tissues and organs needed for survival.

Cancer can be classified into five broad categories. Carcinomas are characterized by cells that cover internal and external parts of the body such as lung, breast, and colon cancer. Sarcomas are characterized by cells that are located in bone, cartilage, fat, connective tissue, muscle, and other supportive tissues. Lymphomas are cancers that begin in the lymph nodes and immune system tissues. Leukemias are cancers that begin in the bone marrow and often accumulate in the bloodstream. Adenomas are cancers that arise in the thyroid, the pituitary gland, the adrenal gland, and other glandular tissues.

Treatment for colorectal cancer can involve surgery, systemic therapy, therapies in interventional radiology, radiation therapy and/or immunotherapy. Some patients may access one or more of these therapies in the management of their disease. For Colorectal cancers, standard chemotherapy regimens have included—Capecitabine (Xeloda™), Fluorouracil (95FU), Folinic Acid (Leucovorin), Irinotecan (Camptosar™), Mitomycin C, Oxaliplatin (Eloxatin™), Raltitrexed (Tomudex™) and Tegafur-Uracil (UFT).

Almost 40% of colorectal tumors harbor KRAS mutations; more than 60% of non small cell lung tumors harbor KRAS mutations, and almost 45% of multiple myeloma patients harbor NRAS mutations. In the RAS mutant cancers, chemotherapeutic options can be highly ineffective. Presence of RAS mutation in cancer indicates a poor prognosis and a low likelihood of successful treatment.

The Ras-Raf-MEK-ERK or mitogen-activated protein kinase (MAPK) pathway is a major signaling cascade in the cell that can be involved in cellular proliferation and differentiation. Ras is a small GTPase protein attached in the cell membrane via prenylation and palmitoylation, which are post-translational modifications important for RAS function.

Generally, RAS acts downstream of receptor tyrosine kinases, which are responsive to a variety of factors, including growth factors. c-MET is an example of a receptor tyrosine kinase, where upon binding of hepatocyte growth factor (HGF), the MAPK signaling cascade is initiated. Additionally, MET is often overexpressed in tumors with mutant RAS possibly due to the fact that MET is a transcriptional target for some of the effectors downstream of the MAPK pathway. Upon binding of a mitogen to c-Met, RAS hydrolyzes GTP allowing for activation and membrane recruitment and activation of RAF.

RAF is a serine/threonine kinase and the main initiator of the MAPK pathway. Regulation of RAF is tightly controlled by a series of phosphatases, kinases, and binding partners. Upon activation, RAF phosphorylates MEK, which in turn phosphorylates ERK. ERK is then able to activate many transcription factors, allowing for transcription and translation of proteins involved in cellular proliferation and differentiation.

HMG-CoA reductase is the rate limiting enzyme for cholesterol synthesis via the mevalonate pathway. It is also involved in the synthesis of other sterols, isoprenoids, and lipids. ATP citrate lyase is further upstream of HMG-CoA reductase in the lipid synthesis pathway. ATP citrate lyase catalyzes the conversion of citrate to acetyl CoA, an important precursor for lipogenesis.

Two examples of isoprenoids synthesized by HMG-CoA reductase- and ATP citrate lyase-mediated activity include farnesyl and geranylgeranyl groups. Isoprenoids can be added to proteins as a post-translational modification in a process known as isoprenylation.

Farnesyl groups are conjugated to proteins containing a CaaX (wherein a is any aliphatic amino acid) motif at the C-terminus of the protein. This occurs through the activity of specific farnesyltransferases (FTases). FTases catalyze the reaction between farnesyl diphosphate and the protein to allow conjugation of the farnesyl group to the cysteine contained within the protein. One of the targets of FTase includes RAS.

Geranylgeranyl groups are conjugated to proteins via the activity of a geranylgeranyl transferase (GGTase). The GGTase catalyzes the conjugation of the geranylgeranyl group onto the consensus site CaaL (where a is any aliphatic amino acid) at the C-terminus of specific proteins. RAS can also be geranylgeranylated.

In some embodiments, the present disclosure provides a composition comprising a combination of a Raf inhibitor or a c-MET inhibitor; and a HMG-CoA reductase inhibitor or ATP citrate lyase inhibitor or farnesyl diphosphate synthase inhibitor or farnesyltransferase inhibitor or geranylgeranyl transferase inhibitor; and optionally a pharmaceutically acceptable excipient.

In some embodiments, the RAF inhibitor and c-Met inhibitor belong to a class of anti-cancer agents.

In some embodiments, the HMG-CoA reductase inhibitor, ATP citrate lyase inhibitor, farnesyl diphosphate synthase inhibitor, farnesyltransferase inhibitor and feranylgeranyl transferase inhibitor belong to a class of prenylation inhibitors.

In some embodiments, the RAF inhibitor is Sorafenib, Vemurafenib, GDC-0879, PLX-4720, Regorafenib, RAF265, NVP-BHG712, SB590885, ZM 336372, AZ628, or a pharmaceutically-acceptable salt thereof or any combination thereof.

In some embodiments, the c-MET inhibitor is ARQ-197, Crizotinib, PF-04217903, JNJ38877605, PHA-665752, SUI1274, INCB28060, AMG-208, NVP-BVU972, BMS-777607, SGX-523, or a pharmaceutically-acceptable salt thereof or a combination thereof.

In some embodiments, the HMG-CoA reductase inhibitor is simvastatin, atorvastatin, cerivastatin, fluvastatin, lovastatin, mevastatin, pitavastatin, pravastatin, rosuvastatin, a pharmaceutically-acceptable salt thereof or any combination thereof.

In some embodiments, the ATP citrate lyase inhibitor is SB 204990, SB 201076, ETC-1002, BMS-303141, a pharmaceutically-acceptable salt thereof or any combination thereof.

In some embodiments, the Farnesyltransferase inhibitor is Tipifamib, Lonafamib, BMS-214662, L778123, L-731734, B1086, L-744832, BIM-46228, FTI-276, RPR-130401, FTI-2148, FTI-2628, FTI-277, FTase Inhibitor I, FTase Inhibitor II, FTase Inhibitor III, L-745631, L-739749, a pharmaceutically-acceptable salt thereof or any combination thereof.

In some embodiments, the Geranylgeranyltransferase inhibitor is GGTI-2418, GGTI-2133, GGTI-2147, GGTI-2154, GGTI-2166, GGTI-286, GGTI-287, GGTI-297, GGTI-298, a pharmaceutically-acceptable salt thereof or any combination thereof.

In some embodiments, the Farnesyl diphosphate synthatase inhibitor is Zoledronate, Risedronate, Ibandronate, Alendronate, Pamidronate, Etidronate, Clodronate, Neridronate, Tiludronate, Incadronate, Olpadronate, EB-1053, Minodronate, a pharmaceutically-acceptable salt thereof, or any combination thereof.

In some embodiments the RAF inhibitor is Sorafenib, the c-MET inhibitor is ARQ-197 or Crizotinib, the HMG-CoA reductase inhibitor is simvastatin or atorvastatin or rosuvastatin, the ATP citrate lyase inhibitor is SB 204990 or BMS303141, the Farnesyltransferase inhibitor is Tipifamib, the Geranylgeranyltransferase inhibitor is GGTI-298, and Farnesyl diphosphate synthatase inhibitor is Risedronate.

The present disclosure relates to a kit comprising a combination of a Raf inhibitor or a c-MET inhibitor; and a HMG-CoA reductase inhibitor or ATP citrate lyase inhibitor or Farnesyl diphosphate synthase inhibitor or Farnesyltransferase inhibitor or Geranylgeranyl transferase inhibitor; and optionally a pharmaceutically acceptable excipient, wherein the kit comprises one or a plurality of dosage forms.

The present disclosure further relates to a method for treating cancer and associated conditions in a subject in need or want of relief thereof, the method comprising administering to the subject a combination of: a therapeutically-effective amount of a Raf inhibitor or a c-MET inhibitor; and a therapeutically-effective amount of a HMG-CoA reductase inhibitor or ATP citrate lyase inhibitor or Farnesyl diphosphate synthase inhibitor or Farnesyltransferase inhibitor or Geranylgeranyl transferase inhibitor and wherein the administration uses one or a plurality of dosage forms, each dosage form comprising one or more inhibitors, and wherein each dosage form optionally further comprises a pharmaceutically-acceptable excipient.

The present disclosure further relates to a use of a combination of compounds in the preparation of a medicament for the treatment of cancer and associated conditions, the compounds comprising: a Raf inhibitor or a c-MET inhibitor; and a HMG-CoA reductase inhibitor or ATP citrate lyase inhibitor or Farnesyl diphosphate synthase inhibitor or Farnesyltransferase inhibitor or Geranylgeranyl transferase inhibitor.

The present disclosure further relates to a combination of compounds for use in the treatment of cancer and associated conditions, the compounds comprising: a Raf inhibitor or a c-MET inhibitor; and a HMG-CoA reductase inhibitor or ATP citrate lyase inhibitor or Farnesyl diphosphate synthase inhibitor or Farnesyltransferase inhibitor or Geranylgeranyl transferase inhibitor.

The present disclosure also relates to a process of preparing a composition described herein, said process comprising: combining a Raf inhibitor or a c-MET inhibitor; and a HMG-CoA reductase inhibitor or a ATP citrate lyase inhibitor or a Farnesyl diphosphate synthase inhibitor or a Farnesyltransferase inhibitor or a Geranylgeranyl transferase inhibitor, optionally along with a pharmaceutically acceptable excipient in any ratio, any concentration, or any order thereof.

In some embodiments, the RAF inhibitor and c-Met inhibitor belong to a class of anti-cancer agents.

In some embodiments, the HMG-CoA reductase inhibitor, ATP citrate lyase inhibitor, Farnesyl diphosphate synthase inhibitor, Farnesyltransferase inhibitor, and Geranylgeranyl transferase inhibitor belong to a class of prenylation inhibitors.

In some embodiments, the RAF inhibitor is Sorafenib, Vemurafenib, GDC-0879, PLX-4720, Regorafenib, RAF265, NVP-BHG712, SB590885, ZM 336372, AZ628, or a pharmaceutically-acceptable salt of any of the foregoing, or any combination thereof.

In some embodiments, the c-Met inhibitor is ARQ-197, CRIZOTINIB, PF-04217903, JNJ38877605, PHA-665752, SUI1274, INCB28060, AMG-208, NVP-BVU972, BMS-777607, SGX-523, or a pharmaceutically-acceptable salt of any of the foregoing, or a combination thereof.

In some embodiments, the HMG-CoA reductase inhibitor is simvastatin, atorvastatin, cerivastatin, fluvastatin, lovastatin, mevastatin, pitavastatin, pravastatin, rosuvastatin, a pharmaceutically-acceptable salt of any of the foregoing, or any combination thereof.

In some embodiments, the ATP citrate lyase inhibitor is SB 204990, SB 201076, ETC-1002, BMS-303141, a pharmaceutically-acceptable salt of any of the foregoing, or any combination thereof.

In some embodiments, the Farnesyltransferase inhibitor is Tipifamib, Lonafamib, BMS-214662, L778123, L-731734, B1086, L-744832, BIM-46228, FTI-276, RPR-130401, FTI-2148, FTI-2628, FTI-277, FTase Inhibitor I, FTase Inhibitor II, FTase Inhibitor III, L-745631, L-739749, a pharmaceutically-acceptable salt of any of the foregoing, or any combination thereof.

In some embodiments, the Geranylgeranyltransferase inhibitor is GGTI-2418, GGTI-2133, GGTI-2147, GGTI-2154, GGTI-2166, GGTI-286, GGTI-287, GGTI-297, GGTI-298, a pharmaceutically-acceptable salt of any of the foregoing, or any combination thereof.

In some embodiments, the Farnesyl diphosphate synthatase inhibitor is Zoledronate, Risedronate, Ibandronate, Alendronate, Pamidronate, Etidronate, Clodronate, Neridronate, Tiludronate, Incadronate, Olpadronate, EB-1053, Minodronate, a pharmaceutically-acceptable salt of any of the foregoing, or any combination thereof.

In some embodiments, the Raf inhibitor is Sorafenib, the c-MET inhibitor is ARQ-197 or Crizotinib, the HMG-CoA reductase inhibitor is simvastatin or atorvastatin or rosuvastatin, the ATP citrate lyase inhibitor is SB 204990 or BMS-303141, the Farnesyltransferase inhibitor is Tipifamib, the Geranylgeranyltransferase inhibitor is GGTI-298 and Farnesyl diphosphate synthatase inhibitor is Risedronate.

In some embodiments, the condition is any cancer with a RAS mutation. Non-limiting examples of such cancers include pancreatic cancer, colorectal cancer, lung cancer, brain cancer, head and neck cancer, breast cancer, multiple myeloma, acute non lymphocytic leukemia or myelodysplasia, or any combination of conditions thereof, or any condition having a mutation therein.

In some embodiments, the mutation is a RAS gene mutation or RAS gene mutation in combination with other gene mutations that support cancer. Non-limiting examples of other gene mutations include BRAF, EGFR, B-catenin, CDKN2A, P13KCA, TP53, APC, MYC, BCL2, SOCS1 and SMAD4 or any combination thereof.

The present disclosure further relates to a composition comprising: a) a combination of: i) a compound selected from: a compound of formula I, a compound of formula II and a compound of formula III; or a compound selected from: a compound of formula IV, a compound of formula V and a compound of formula VI; and ii) a compound selected from: a compound of formula VII, a compound of formula VIII, a compound of formula IX, a compound of formula X, a compound of formula XI, a compound of formula XII, a compound of formula XIII, a compound of formula XIV and a compound of formula XV; and b) optionally a pharmaceutically acceptable excipient.

In some embodiments, the compound of formula I is:

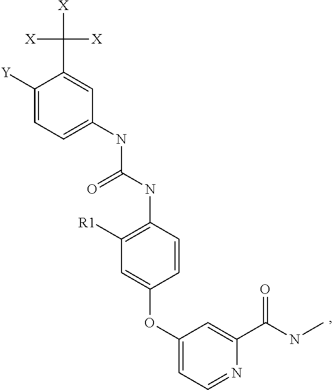

wherein, R1 is hydrogen or halogen; each of X and Y is independently a halogen; and the halogen of R1, X, and Y is independently selected from fluorine, chlorine, bromine, iodine, and astatine.

In some embodiments, the compound of formula II is:

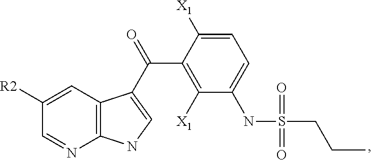

wherein R2 is halogen or halophenyl; X1 is halogen; and the halogen of R2 and X1 is each independently selected from fluorine, chlorine, bromine, iodine, and astatine.

In some embodiments, the compound of formula III is:

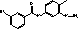

wherein, R3 is (CH₃)₂N— or

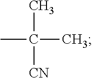

### and R4 is

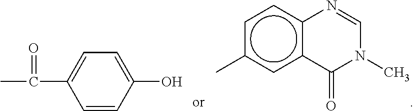

In some embodiments, the compound of formula IV is:

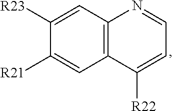

wherein, R21 is selected from

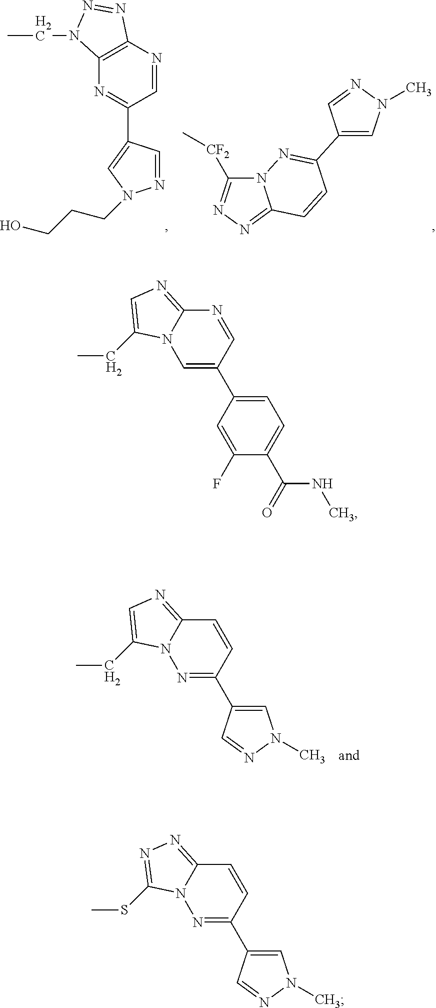

R22 is selected from hydrogen,

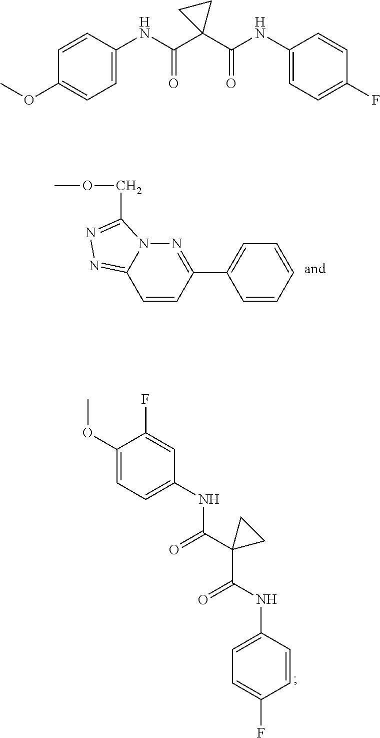 [SMILES: COc1ccc(NC(=O)C2(C(=O)Nc3ccc(F)cc3)CC2)cc1 ; COCc1nnc2ccc(-c3ccccc3)nn12 ; COc1ccc(NC(=O)C2(C(=O)Nc3ccc(F)cc3)CC2)cc1F]

and
R23 is selected from a hydrogen, hydroxy alkoxy and

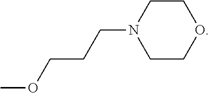

In some embodiments, the compound of formula V is:

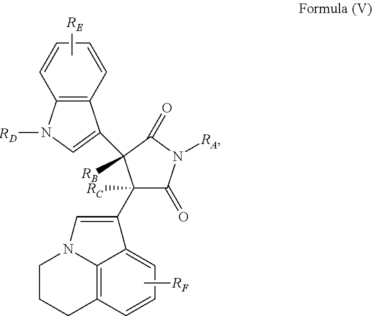

wherein each of R_{A}, R_{B}, R_{C}, R_{D}, R_{E} and R_{F} is independently selected from hydrogen, C₁₋₆alkyl, C₁₋₆aryl, C₁₋₆alkoxy, halogen, hydroxyl and ammonium or any substituted forms thereof.

In some embodiments, the compound of formula VI is:

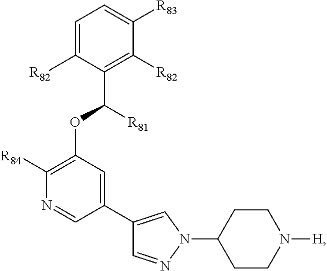

wherein, R81 is selected from hydrogen, alkyl and alkoxy; R82 and R83 are independently chlorine, fluorine, bromine, iodine, or astatine; and R84 is amino or alkylamino.

In some embodiments, the compound of formula VII is:

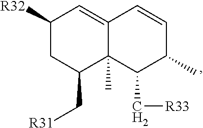

wherein, R31 is selected from a

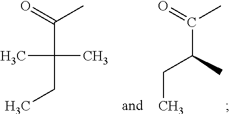

R32 is selected from hydrogen, alkyl and hydroxy; and R33 is selected from

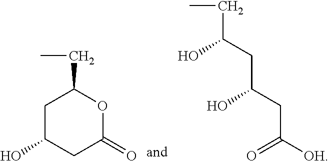

In some embodiments, the compound of formula VIII is:

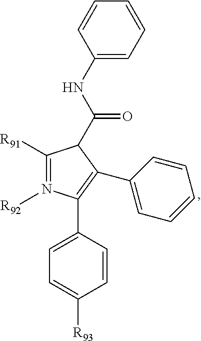

wherein R91 is selected from methyl, ethyl, propyl, isopropyl, butyl, and isobutyl; R92 is

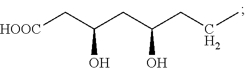

and R93 is selected from fluorine, chlorine, bromine, iodine, and astatine.

In some embodiments, the compound of formula IX is:

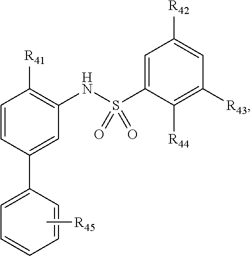

wherein R₄₁ is selected from C₁₋₆alkyl, C₁₋₆alkoxy, hydroxyl and hydrogen; each of R42 and R43 is independently selected from hydrogen, halogen, hydroxy, C₁₋₆alkyl and C₁₋₆alkoxy; R44 is selected from hydrogen, halogen, C₁₋₆alkyl, C₁₋₆alkoxy and hydroxy; and R45 is selected from hydrogen, halogen, C₁₋₆alkyl, C₁₋₆alkoxy and hydroxy.

In some embodiments, the compound of formula X is:

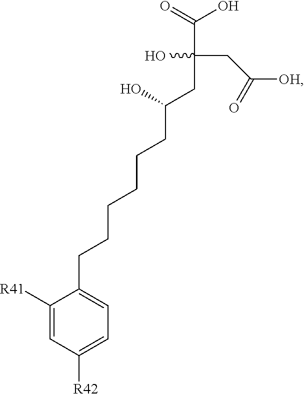

wherein, each of R41 and R42 is independently selected from hydrogen and halogen.

In some embodiments, the compound of formula XI is:

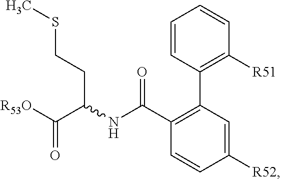

wherein R51 is hydrogen or alkyl; R52 is selected from

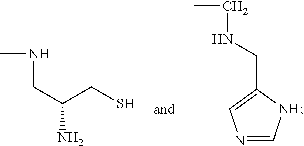

and R53 is selected from hydrogen, benzyl and alkyl.

In some embodiments, the compound of formula XII is:

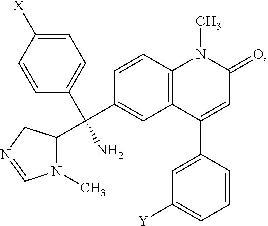

wherein each of X and Y is independently fluorine, chlorine, bromine, iodine, or astatine.

In some embodiments, the compound of formula XIII is:

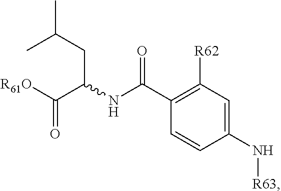

wherein R61 is hydrogen or alkyl; R62 is selected from

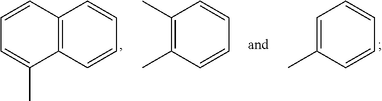

and R63 is selected from

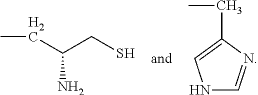

In some embodiments, the compound of formula XIV is:

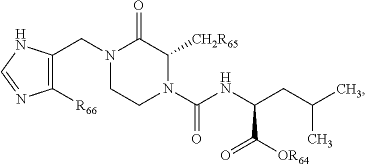

wherein R64 is selected from hydrogen and C₁₋₆alkyl; R65 is selected from

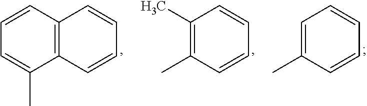

and R66 is selected from hydrogen, hydroxy, C₁₋₆alkyl, C₁₋₆alkoxy and halogen.

In some embodiments, the compound of formula XV is:

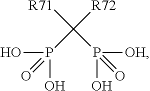

wherein R71 is selected from hydrogen, hydroxyl and halogen; and R72 is selected from alkyl, halogen,

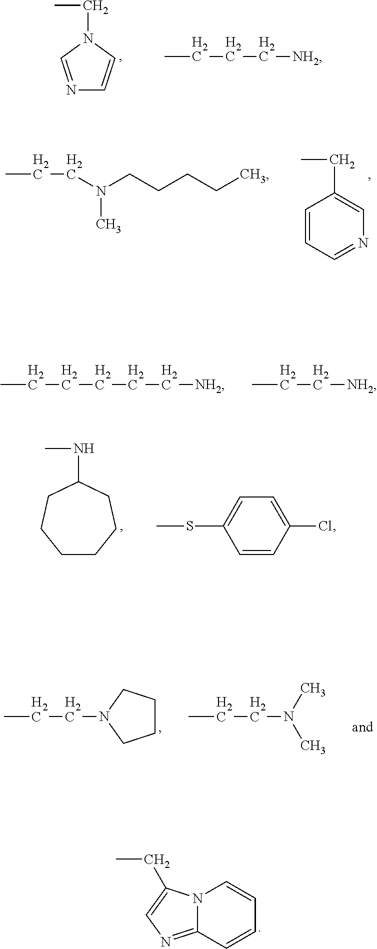

In some embodiments, the composition comprises a combination of: i) a compound of formula I with a compound selected from a compound of formula VIII and a compound of formula IX; or ii) a compound of formula V or a compound of formula VI with a compound selected from a compound of formula VIII and a compound of formula IX; and optionally a pharmaceutically acceptable excipient.

The present disclosure further provides a kit comprising: a) a combination of: i) a compound selected from a compound of formula I, a compound of formula II and a compound of formula III or a compound selected from a compound of formula IV, a compound of formula V and a compound of formula VI; and ii) a compound selected from a compound of formula VII, a compound of formula VIII, a compound of formula IX, a compound of formula X, a compound of formula XI, a compound of formula XII a compound of formula XIII, a compound of formula XIV and a compound of formula XV; and b) optionally a pharmaceutically acceptable excipient, wherein the kit comprises one or a plurality of dosage forms.

In some embodiments, the kit comprises a combination of a compound of formula I with a compound selected from a compound of formula VIII and a compound of formula IX; or a compound of formula V or compound of formula VI with a compound selected from a compound of formula VIII and a compound of formula IX; and optionally a pharmaceutically acceptable excipient, wherein the kit comprises one or a plurality of dosage forms.

The present disclosure further relates to a method for treating cancer and associated conditions in a subject in need of relief thereof, the method comprising administering to the subject a combination of a therapeutically-effective amount of a compound selected from a compound of formula I, a compound of formula II and a compound of formula III or a compound selected from a compound of formula IV, a compound of formula V, and a compound of formula VI; and a therapeutically effective amount of a compound selected from a a compound of formula VII, a compound of formula VIII, a compound of formula IX, a compound of formula X, a compound of formula XI, a compound of formula XII, a compound of formula XIII, a compound of formula XIV, and a compound of formula XV; and wherein the administration uses one or a plurality of dosage forms, each dosage form comprising one or more inhibitors, and wherein each dosage form optionally further comprises a pharmaceutically acceptable excipient.

In some embodiments, the method comprises a combination of: a therapeutically effective amount of a compound of formula I or a compound of formula V or compound of formula VI; and a therapeutically effective amount of a compound of formula VIII or a compound of formula IX; and wherein the administration uses one or a plurality of dosage forms, each dosage form comprising one or more inhibitors, and wherein each dosage form optionally further comprises a pharmaceutically acceptable excipient.

The present disclosure further relates to a use of a combination of compounds in the preparation of a medicament for the treatment of cancer and associated conditions, said combination comprising a compound selected from a compound of formula I, a compound of formula II and a compound of formula III or a compound selected from a group comprising a compound of formula IV, or a compound of formula V and a compound of formula VI with a compound selected from a compound of formula VII, a compound of formula VIII, a compound of formula IX, a compound of formula X, a compound of formula XI, a compound of formula XII, a compound of formula XIII, a compound of formula XIV and a compound of formula XV.

In some embodiments, the method comprises a combination of: a) a compound of formula I with a compound selected from a group comprising a compound of formula VIII and compound of formula IX; or b) a compound of formula V or compound of formula VI with a compound selected from a group comprising compound of formula VIII and compound of formula IX.

The present disclosure further relates to a combination of compounds for use in the treatment of cancer and associated conditions, said combination comprising a compound selected from compounds of formula I, compounds of formula II and compounds of formula III or a compound selected from compounds of formula IV, compounds of formula V and compounds of formula VI with a compound selected from compounds of formula VII, compounds of formula VIII, compounds of formula IX, compounds of formula X, compounds of formula XI, compounds of formula XII, compounds of formula XIII, compounds of formula XIV and compounds of formula XV.

In some embodiments, the combination is a compound of formula I with a compound selected from compounds of formula VIII and compounds of formula IX; or b) a compound of formula V or compounds of formula VI with a compound selected from a group comprising compounds of formula VIII and compounds of formula IX.

The present disclosure further relates to a process of preparing the composition, said process comprising combining a compound selected from compounds of formula I, compounds of formula II and compounds of formula III or compounds selected from compounds of formula IV, compounds of formula V and compounds of formula VI with a compound selected from compounds of formula VII, compounds of formula VIII, compounds of formula IX, compounds of formula X, compounds of formula XI, compounds of formula XII, compounds of formula XIII, compounds of formula XIV and compounds of formula XV, optionally along with pharmaceutically acceptable excipients in any ratio, any concentration or any order thereof.

In some embodiments, the inhibitors or the compounds are present in a dose not exceeding their maximum tolerated dose, for example, in a human, such as a lethal or toxic dose.

In some embodiments, the excipient is selected from granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, and plant cellulosic material and spheronization agents, or any combination thereof.

In some embodiments, the present disclosure provides two-drug combinations, which provide a multi-targeted combinatorial therapeutic approach to treat symptoms associated with RAS mutant cancers and associated conditions. The drug combinations were validated using a Virtual Tumor Cell Platform as described herein.

In some embodiments, the present disclosure provides therapy for RAS mutant tumors irrespective of tissue subtype or origin, with a RAS mutation being a significant factor in disease pathogenesis. One aspect of the present disclosure is to study the biochemical dynamics of cancers carrying mutations in RAS isoforms and design therapies that are effective in this aggressive subset of cancers that are challenging to treat and difficult to survive.

The two-drug combinations of the present disclosure provide synergistic efficacy on the end-point markers where dosing is as low as ¼, ⅓ or ½ of the recommended therapeutic dose of the drug in humans. Using a lower dose of the individual drug also provides an advantage in terms of minimizing the intensity of side-effects or toxicities associated with the drugs. Also, the drug combination works by inhibiting multiple targets minimally, so that an amplified effect is observed on all of the primary end-point markers and at the same time ensuring that all the targets have primary response ability, so as to negate the possibility of immune suppression and secondary infections. Use of smaller doses of individual drugs also lowers the cost of manufacture and formulation, providing an improved effect at a lower price to the subject. Smaller doses can mitigate against wasteful administration of a drug to a physiological system that has been saturated or has reached a peak therapeutic response from smaller, synergistic doses.

In some embodiments, the present disclosure provides a composition comprising a combination of a Raf inhibitor and a 3-hydroxyl-3-methylglutaryl coenzyme A (HMG-CoA) reductase inhibitor or an ATP citrate lyase (ACLY) inhibitor or a Farnesyl diphosphate synthase (FDPS) inhibitor or a Farnesyltransferase (FTase) inhibitor or a Geranylgeranyl transferase (GGTase) inhibitor along with pharmaceutically acceptable excipients.

In some embodiments, the present disclosure provides a kit comprising a composition of a Raf inhibitor and a 3-hydroxyl-3-methylglutaryl coenzyme A (HMG-CoA) reductase inhibitor or an ATP citrate lyase (ACLY) inhibitor or a Farnesyl diphosphate synthase (FDPS) inhibitor or a Farnesyltransferase (FTase) inhibitor or a Geranylgeranyl transferase (GGTase) inhibitor along with pharmaceutically acceptable excipients, wherein the kit comprises one or a plurality of dosage forms.

In some embodiments, the present disclosure provides a composition comprising a combination of a c-MET inhibitor and a 3-hydroxyl-3-methylglutaryl coenzyme A (HMG-CoA) reductase inhibitor or an ATP citrate lyase (ACLY) inhibitor or a Farnesyl diphosphate synthase (FDPS) inhibitor or a Farnesyltransferase (FTase) inhibitor or a Geranylgeranyl transferase (GGTase) inhibitor along with pharmaceutically acceptable excipients.

In some embodiments, the present disclosure provides a kit comprising a composition of a c-MET inhibitor and a 3-hydroxyl-3-methylglutaryl coenzyme A (HMG-CoA) reductase inhibitor or an ATP citrate lyase (ACLY) inhibitor or a Farnesyl diphosphate synthase (FDPS) inhibitor or a Farnesyltransferase (FTase) inhibitor or a Geranylgeranyl transferase (GGTase) inhibitor along with pharmaceutically acceptable excipients, wherein the kit comprises one or a plurality of dosage forms.

In some embodiments, the CW137 and CW147 class of drugs are anti-cancer agents and function as Raf inhibitors and c-MET inhibitors, respectively.

In some embodiments, the CW231 class of drugs are prenylation inhibitors and function as HMG-CoA reductase inhibitors or ATP citrate lyase inhibitors or Farnesyl diphosphate synthase inhibitors or Farnesyltransferase inhibitors or Geranylgeranyl transferase inhibitors.

The CW137 class of drugs are kinase inhibitors with RAF1 as a primary target. In cells harboring RAS mutations (KRAS/NRAS/HRAS), MEK/ERK signaling is hyper-activated through the RAS/RAF/ERK pathway. Inhibition of RAF1 by CW137 can bring about a reduction in MEK/ERK signaling in cells with mutated RAS. Inhibition by CW137 can also result in the reduction of NFκB signaling via RAF. Thus, CW137 can lead to a reduction in proliferation and viability of the tumor cell. Other targets of CW137 include RTKs including FLT1, FLT4, PDGFRB and KDR, inhibition of which can cause a reduction in angiogenesis apart from impacting viability and proliferation.

The CW137 representative class of drugs is a kinase inhibitor having potent inhibition activity against RAF kinase. Apart from RAF kinase, CW137 can also inhibit KDR, FLT1, FLT4, PDGFRA, PDGFRB, and KIT kinases. In a biochemical assay, the activity of CW137 was as shown in TABLE 1.

TABLE 1

| Target | IC₅₀ (μM) |
|---|---|
| Raf-1 | 0.006 |
| BRAF wild-type | 0.022 |
| V599E BRAF mutant | 0.038 |
| VEGFR-2 | 0.09 |
| mVEGFR-2 (flk-1) | 0.015 |
| mVEGR-3 | 0.02 |
| mPDGFR-B | 0.057 |
| Flt-3 | 0.058 |
| c-KIT | 0.07 |
| FGFR-1 | 0.58 |
| ERK-1, MEK-1, EGFR, HER-2, IGFR-1, c-met, PKB, | >10 |
| PKA, cdk1/cyclinB, PKC-alpha, PKC-gamma, pim-1 | |

In patients, the steady state plasma level concentration of CW137 has been found in the range of 15-20 μM. At such high concentrations, the on-target inhibition of all of the above listed targets can be very high. Such high inhibition, especially of VEGF receptor signaling, can lead to poorly controlled hypertension, reduced capillary density, contractile dysfunction, fibrosis, and heart failure in extreme cases.

However, much lower doses could show a therapeutic benefit in KRAS mutant patients where tumorigenesis is majorly driven by the RAS-RAF-MEK-ERK pathway. In patients with KRAS mutations, much lower doses of CW137 can inhibit tumor development and progression by effective down-regulation of MAPK signaling.

In A549 cells, which are KRAS mutant, ERK is one of the major components driving tumorogenesis. Inhibition of RAF by CW137 at a low concentration results in a reduction in viability of the cells. However, in H1650 cells (KRAS wild type), a similar reduction in ERK and RAF1 is not able to reduce viability.

Use of CW137 at a lower dose can provide significant efficacy in patients with a KRAS mutation over those who are KRAS wild type, while bringing down CW137 mediated toxicity.

The CW147 class of drugs are inhibitors of MET, which is found to be overexpressed in multiple cancers, especially in cancers harboring RAS mutations. Transcription of MET is activated by transcription factors such as AP1, ETS1, SP1 and HIF most of which are downstream of ERK signaling. Thus, in RAS mutant cells, high ERK signaling can lead to a high expression of MET. Overexpressed or dysregulated MET can induce hyperproliferation, an increase in viability, increased angiogenesis and invasion of cancer cells by activation of different oncogenes like RAS, AKT, and SRC. Additionally, stromal cells in the tumor microenvironment expressHGF, which can activate MET receptors on tumor cells and potentially cause those cells to gain resistance to molecularly targeted drugs. CW147 inhibits MET signaling and can decrease MET-mediated RAS, PI3K and SRC activation.

The CW147 class of drugs also include multikinase inhibitors that can also inhibit c-MET. In an enzymatic assay, Crizotinib, one of the compounds of this class, can inhibit 13 different kinases. In cellular assays, crizotinib demonstrated potent inhibition of c-MET/ALK, with a 10-fold selectivity window for RON. The cellular IC₅₀ values for the different kinases are shown in TABLE 2.

TABLE 2

| | c-MET | ALK | RON | AXL | TIE2 | TRKA | TRKB | ABL | IR | LCK |
|---|---|---|---|---|---|---|---|---|---|---|
| Cell IC₅₀ | 8 | 20 | 80 | 294 | 448 | 580 | 399 | 1159 | 2887 | 2741 |
| (nM) | | | | | | | | | | |

The CW231 class of drugs includes 3-hydroxy-3-methylglutaryl coenzyme A (HMG-CoA) reductase inhibitors, ATP Citrate lyase (ACLY) inhibitors, Farnesyltransferase Inhibitors, Geranylgeranyltransferase inhibitors or Farnesyl diphosphate synthatase inhibitors. Collectively these are the inhibitors of prenylation process. By inhibiting HMG-CoA reductase or ATP Citrate lyase (ACLY) or Farnesyltransferase or Geranylgeranyltransferase or Farnesyl diphosphate synthatase, CW231 inhibits the process of prenylation, a post-translational modification of many GTPases.

Prenylation is facilitated by mevalonate-derived prenylgroups, farnesylpyrophosphate (FPP) and geranylgeranylpyrophosphate (GGPP). The process of prenylation facilitates protein-protein/protein-membrane interactions of important proteins including, but not limited to, RAS, RHO, RAC1, CDC42, and RHEB. In the case of RAS mutants, these pathways can become highly activated. RAS can also mediate the activation of RHO, RAC1, CDC42, PI3K and MEK. This mediation can lead to increased tumor growth mediated by over-activated RAS. Therefore, inhibiting the prenylation process can inhibit RAS-mediated tumor growth by decreasing prenylation of RAS as well as its downstream effectors.

In some embodiments, the combination of a compound from the CW137 class and a compound from the CW231 class in any amount, ratio, concentration, or order thereof can inhibit the RAS-RAF-ERK pathway, which can be hyper-activated in RAS mutant cells, via RAF1 inhibition. Further, the above signaling cascade is altered due to the inhibition of the prenylation of RAS. In this way, the combination can hit the RAS signaling pathway at two different points in the cascade and can show a significant reduction in ERK activity and its downstream tumorigenic effects.

Conventionally, the dose range for any of the CW231 compounds is 5-40 mg/day for a patient having a high risk of coronary heart disease (CHD). Higher doses of this drug can also be tolerated for cancer therapy, but carries an increased risk of myopathy. In cancer patients, the drug can be given at doses as high as the maximum tolerated dose of 15 mg/kg/day, which is 25-fold higher than a typical dose. However, at higher doses the drug can inhibit RAS signaling in normal cells leading to serious cytotoxic effects. In the case of a KRAS mutation, where the RAS pathway is the major driver for tumorogenesis, inhibition of prenylation can result in a significant reduction in tumorigenesis even at lower dosages. In contrast, for non-KRAS mutations, where other signaling pathways drive the tumor progression, the minimal inhibition of the prenylation of KRAS with the lower dose of CW231 does not cause a sufficient reduction in tumor viability.

As provided herein, a lower dose of CW137 along with a lower dose of CW231 can provide an enhanced therapeutic effect in KRAS mutant cells by inhibiting the KRAS pathway along with other key proteins of RAS super-family.

In some embodiments, the combination of any compound CW147 and any compound CW231 in any amount, ratio, concentration, or order thereof can provide simultaneous inhibition of the RAS/RAF/ERK pathway by reduced prenylation as a result of mevalonate pathway inhibition and inhibition of the MET signaling pathway, both of which can be hyperactive in cells with a RAS mutation, show an enhanced potential to reduce growth of tumor cells.

In some embodiments, the combination of any compound CW147 and any compound CW231 in any amount, ratio, concentration, or order thereof can provide an enhanced and synergistic therapeutic effect on RAS mutant cells by inhibiting the dominant RAS signaling pathway as well as by removing a possible resistance mechanism exerted by overexpressed MET or by paracrine HGF-MET signaling from stromal cells in the tumor microenvironment.

As used herein, the term, “CW137231,” refers to a combination of any CW137 compound and any CW231 compound in any amount, ratio, concentration, or order thereof.

As used herein, the term, “CW147231,” refers to a combination of any CW147 compound and any CW231 compound in any amount, ratio, concentration, or order thereof.

Non-limiting examples of CW137 include: a) Nexavar™ or Sorafenibtosylate or BAY 43-9006 or 4-[4-[[4-chloro-3-(trifluoromethyl)phenyl]carbamoylamino]phenoxy]-N-methylpyridine-2-carboxamide or a pharmaceutically acceptable salt thereof; b) Vemurafenib PLX4032 Or RG7204 Or Zelboraf or RO5185426 Or N-[3-[5-(4-chlorophenyl)-1H-pyrrolo[2,3-b]pyridine-3-carbonyl]-2,4-difluorophenyl]propane-1-sulfonamide or a pharmaceutically acceptable salt thereof; c) GDC-0879 or 2-[4-[(1E)-1-hydroxyimino-2,3-dihydroinden-5-yl]-3-pyridin-4-ylpyrazol-1-yl]ethanol or a pharmaceutically acceptable salt thereof; d) PLX-4720 Or N-[3-(5-chloro-1H-pyrrolo[2,3-b]pyridine-3-carbonyl)-2,4-difluorophenyl]propane-1-sulfonamide or a pharmaceutically acceptable salt thereof; e) Regorafenib or BAY 73-4506 Or 4-[4-[[4-chloro-3-(trifluoromethyl)phenyl]carbamoylamino]-3-fluorophenoxy]-N-methylpyridine-2-carboxamide or a pharmaceutically acceptable salt thereof; f) RAF265 or CHIR-265 or 1-methyl-5-[2-[5-(trifluoromethyl)-1H-imidazol-2-yl]pyridin-4-yl]oxy-N-[4-(trifluoromethyl)phenyl]benzimidazol-2-amine or a pharmaceutically acceptable salt thereof; g) NVP-BHG712 or 4-methyl-3-[(1-methyl-6-pyridin-3-ylpyrazolo[3,4-d]pyrimidin-4-yl)amino]-N-[3-(trifluoromethyl)phenyl]benzamide or a pharmaceutically acceptable salt thereof; h) SB590885 or GSK2118436 Or N,N-dimethyl-2-[4-[(4Z)-4-(1-nitroso-2,3-dihydroinden-5-ylidene)-5-(1H-pyridin-4-ylidene)-1H-imidazol-2-yl]phenoxy]ethanamine or a pharmaceutically acceptable salt thereof; i) ZM 336372 or 3-(dimethylamino)-N-[3-[(4-hydroxybenzoyl)amino]-4-methylphenyl]benzamide or a pharmaceutically acceptable salt thereof; and j) AZ628 Or 3-(2-cyanopropan-2-yl)-N-[4-methyl-3-[(3-methyl-4-oxoquinazolin-6-yl)amino]phenyl]benzamide or a pharmaceutically acceptable salt thereof.

Non-limiting examples of CW137 include the following:

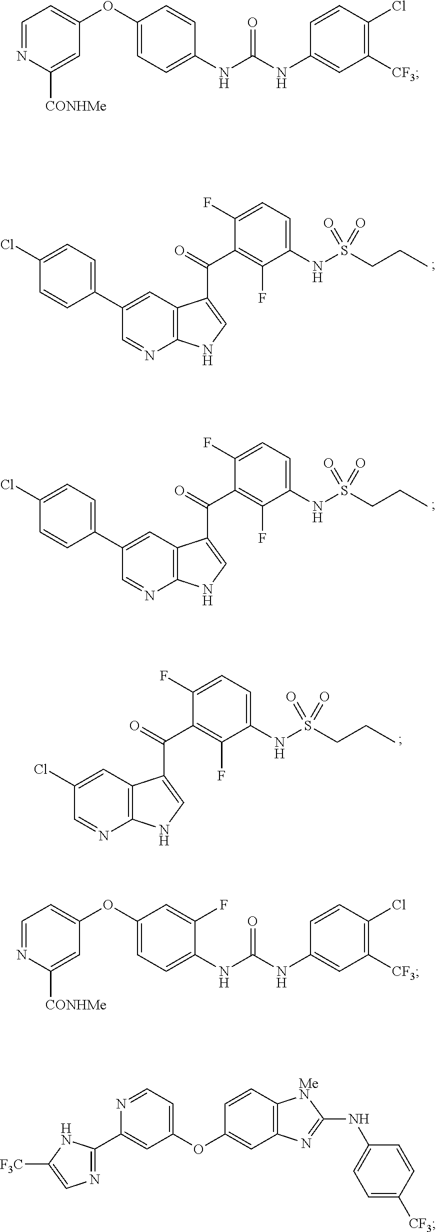

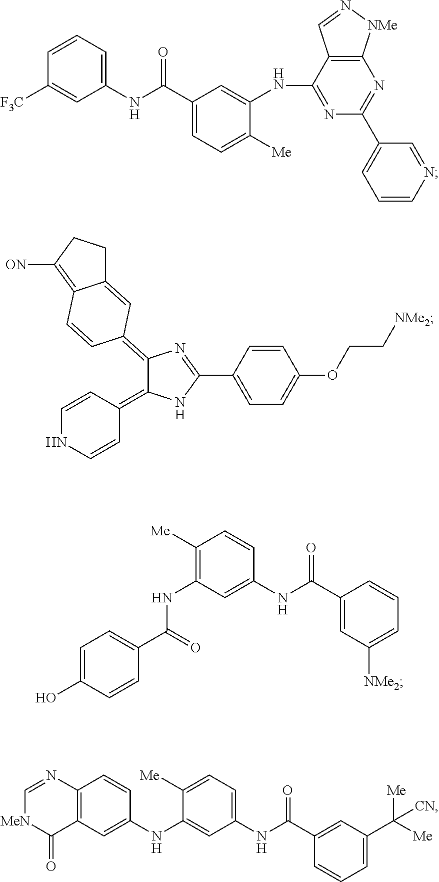

and pharmaceutically-acceptable salts thereof.

In some embodiments, CW137 is a Raf inhibitor represented by a compound of formula (I) or (II) or (III).

In some embodiments, the compound of formula I is:

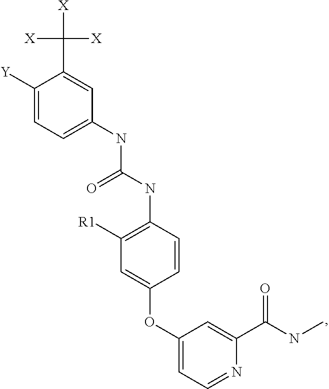

wherein, R1 is hydrogen or halogen; each of X and Y is independently a fluorine, chlorine, bromine, iodine, or astatine.

In some embodiments, the compound of formula II is:

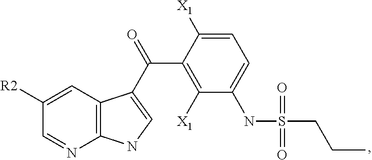

wherein, R2 is halogen or halophenyl; X1 is halogen; and wherein the halogen of R2 and X1 is selected from fluorine, chlorine, bromine, iodine and astatine.

In some embodiments, the compound of formula III is:

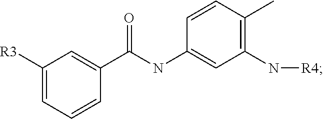

wherein, R3 is (CH₃)₂N- or

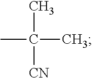

### and R4 is

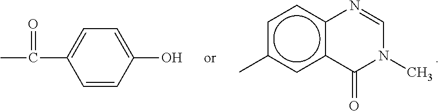

In some embodiments, Raf inhibitor is compound of formula I:

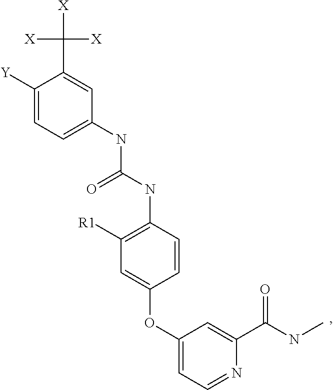

wherein, R1 is hydrogen; and each of X and Y is independently fluorine, chlorine, bromine, iodine, or astatine. In some embodiments, X is fluorine and Y is chlorine.

Non-limiting examples of CW147 include: a) ARQ-197 or Tivantinib™ or (3R,4R)-3-(5,6-dihydro-4H-pyrrolo[3,2,1-ij]quinolin-1-yl)-4-(1H-indol-3-yl)pyrrolidine-2,5-dione or a pharmaceutically acceptable salt thereof; b) PF-04217903 or 2-[4-[3-(quinolin-6-ylmethyl) triazolo[4,5-b]pyrazin-5-yl]pyrazol-1-yl]ethanol or a pharmaceutically acceptable salt thereof; c) JNJ-38877605 Or 6-[difluoro-[6-(1-methylpyrazol-4-yl)-[1,2,4]triazolo[4,3-b]pyridazin-3-yl]methyl]quinoline or a pharmaceutically acceptable salt thereof; d) PHA-665752 or (3Z)-5-[(2,6-dichlorophenyl)methylsulfonyl]-3-[[3,5-dimethyl-4-[(2R)-2-(pyrrolidin-1-ylmethyl)pyrrolidine-1-carbonyl]-1H-pyrrol-2-yl]methylidene]-1H-indol-2-one or a pharmaceutically acceptable salt thereof; e) SU11274 or (3Z)—N-(3-chlorophenyl)-3-[[3,5-dimethyl-4-(4-methylpiperazine-1-carbonyl)-1H pyrrol-2-yl]methylidene]-N-methyl-2-oxo-1H-indole-5-sulfonamide or a pharmaceutically acceptable salt thereof; f) INCB28060 or 2-fluoro-N-methyl-4-[7-(quinolin-6-ylmethyl)imidazo[1,2-b][1,2,4]triazin-2-yl]benzamide or a pharmaceutically acceptable salt thereof; g) AMG-208 or Triazolopyridazine or 7-methoxy-4-[(6-phenyl-[1,2,4]triazolo[4,3-b]pyridazin-3-yl)methoxy]quinoline or a pharmaceutically acceptable salt thereof; h) NVP-BVU972 or 6-[[6-(1-methylpyrazol-4-yl)imidazo[1,2-b]pyridazin-3-yl]methyl]quinoline or a pharmaceutically acceptable salt thereof; i) BMS-777607 or N-[4-(2-amino-3-chloropyridin-4-yl)oxy-3-fluorophenyl]-4-ethoxy-1-(4-fluorophenyl)-2-oxopyridine-3-carboxamide or a pharmaceutically acceptable salt thereof; j) SGX-523 or 6-[[6-(1-methylpyrazol-4-yl)-[1,2,4]triazolo[4,3-b]pyridazin-3-yl]sulfanyl]quinoline or a pharmaceutically acceptable salt thereof; k) Crizotinib or PF-2341066 or Xalkori™ Or PF-02341066 Or PF 2341066 Or 3-[(1R)-1-(2,6-dichloro-3-fluorophenyl)ethoxy]-5-(1-piperidin-4-ylpyrazol-4-yl)pyridin-2-amine Or a pharmaceutically acceptable salt thereof; 1) Cabozantinib or XL184 or BMS-907351 Or 1-N-[4-(6,7-dimethoxyquinolin-4-yl)oxyphenyl]-1-N′-(4-fluorophenyl)cyclopropane-1,1-dicarboxamide or a pharmaceutically acceptable salt thereof; m) Foretinib or GSK 1363089 or XL880 or 1-N′-[3-fluoro-4-[6-methoxy-7-(3-morpholin-4-ylpropoxy)quinolin-4-yl]oxyphenyl]-1-N-(4-fluorophenyl)cyclopropane-1,1-dicarboxamide or a pharmaceutically acceptable salt thereof; n) MGCD265 or N-[[3-fluoro-4-[2-(1-methylimidazol-4-yl)thieno[3,2-b]pyridin-7-yl]oxyphenyl]carbamothioyl]-2-phenylacetamide or a pharmaceutically acceptable salt thereof; o) Golvatinib or E7050 or 1-N′-[2-fluoro-4-[2-[[4-(4-methylpiperazin-1-yl)piperidine-1-carbonyl]amino]pyridin-4-yl]oxyphenyl]-1-N-(4-fluorophenyl)cyclopropane-1,1-dicarboxamide or a pharmaceutically acceptable salt thereof; and p) MK-2461 or 9-[[[(2R)-1,4-dioxan-2-yl]methyl-methylsulfamoyl]amino]-2-(1-methylpyrazol-4-yl)-11-oxobenzo[1,2]cyclohepta[2,4-b]pyridine or a pharmaceutically acceptable salt thereof.

Non-limiting examples of CW147 include the following:

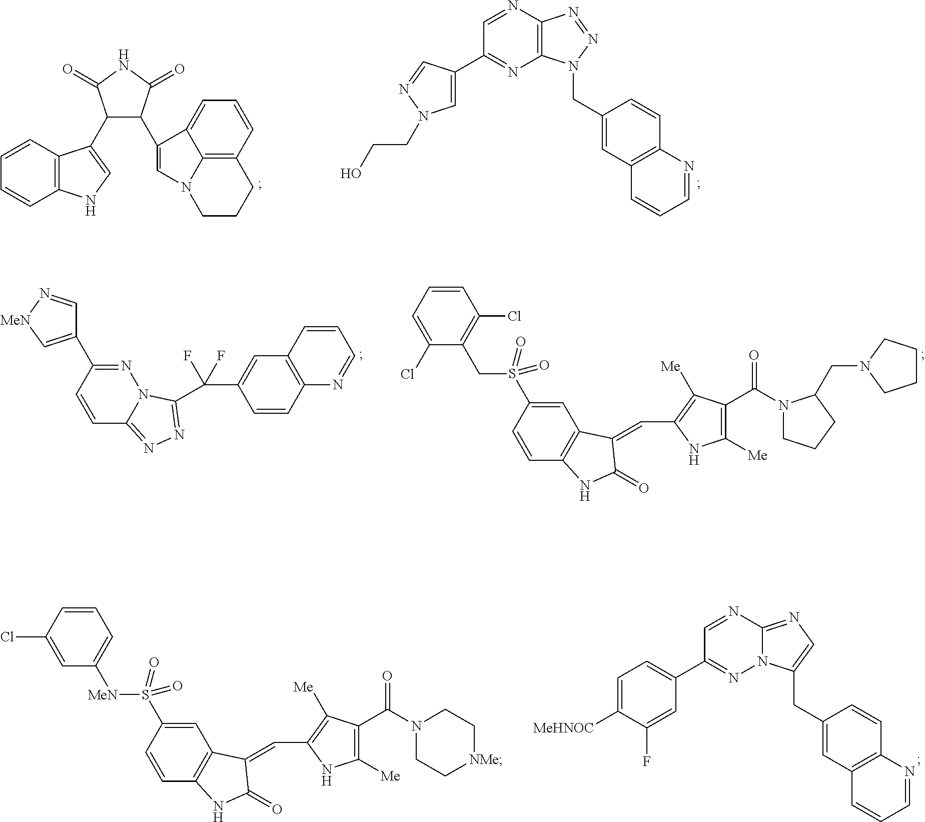

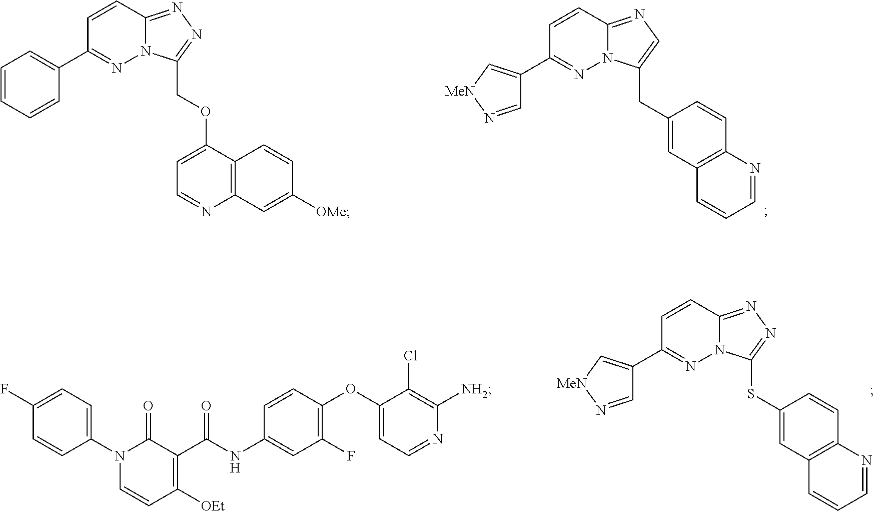 [SMILES: COc1ccc2c(OCc3nnc4ccc(-c5ccccc5)nn34)ccnc2c1 ; Cn1cc(-c2ccc3ncc(Cc4ccc5ncccc5c4)n3n2)cn1 ; CCOc1ccn(-c2ccc(F)cc2)c(=O)c1C(=O)Nc1ccc(Oc2ccnc(N)c2Cl)c(F)c1 ; Cn1cc(-c2ccc3nnc(Sc4ccc5ncccc5c4)n3n2)cn1]

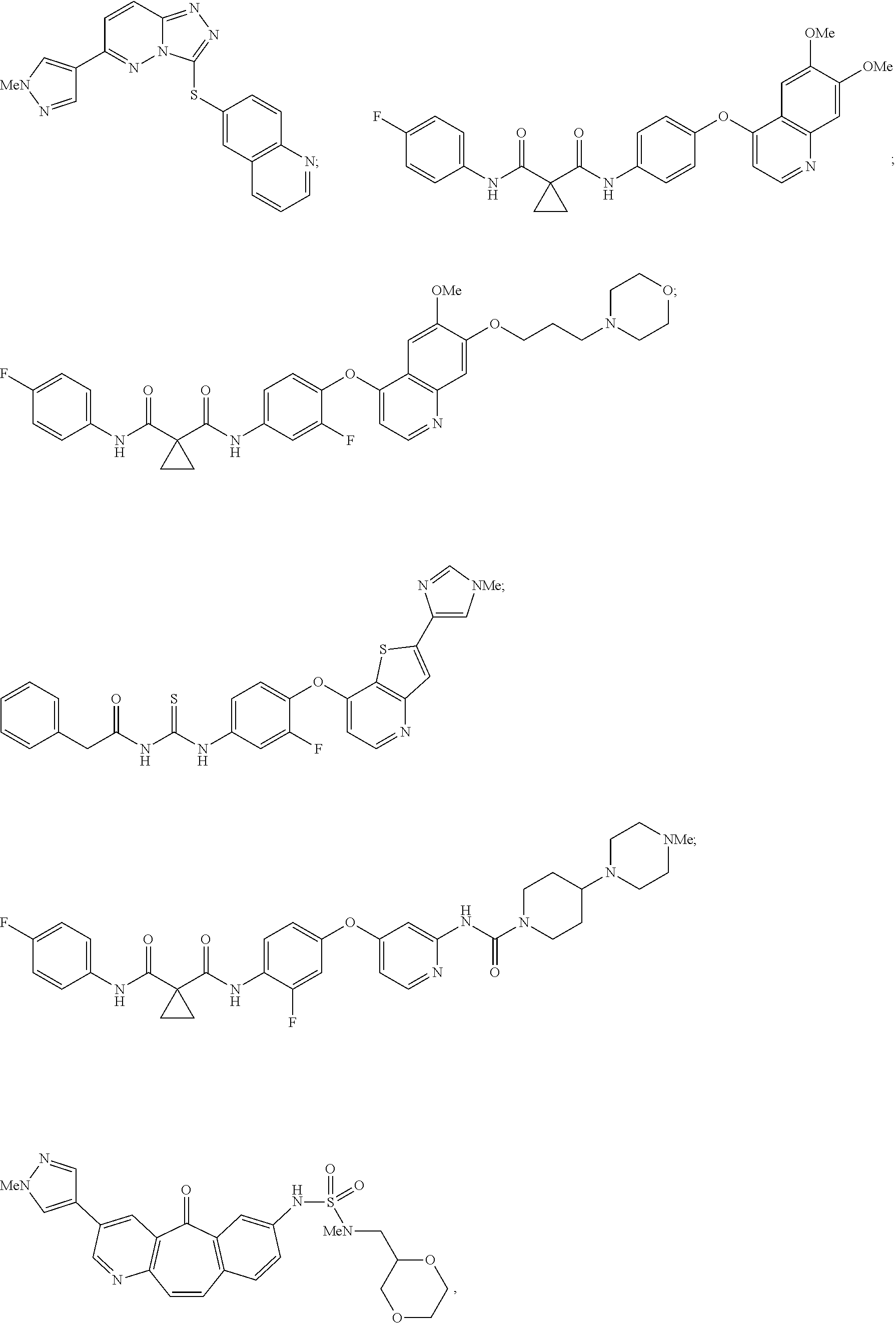 [SMILES: Cn1cc(-c2ccc3nnc(Sc4ccc5ncccc5c4)n3n2)cn1 ; COc1cc2nccc(Oc3ccc(NC(=O)C4(C(=O)Nc5ccc(F)cc5)CC4)cc3)c2cc1OC ; COc1cc2c(Oc3ccc(NC(=O)C4(C(=O)Nc5ccc(F)cc5)CC4)cc3F)ccnc2cc1OCCCN1CCOCC1 ; Cn1cnc(-c2cc3nccc(Oc4ccc(NC(=S)NC(=O)Cc5ccccc5)cc4F)c3s2)c1 ; CN1CCN(C2CCN(C(=O)Nc3cc(Oc4ccc(NC(=O)C5(C(=O)Nc6ccc(F)cc6)CC5)c(F)c4)ccn3)CC2)CC1 ; CN(CC1COCCO1)S(=O)(=O)Nc1ccc2ccc3ncc(-c4cnn(C)c4)cc3c(=O)c2c1]

and pharmaceutically-acceptable salts thereof.

In some embodiments, CW147 is a cMET inhibitor represented by compound of formula IV or V or VI.

In some embodiments, the compound of formula IV is:

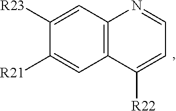

wherein, R21 is selected from hydrogen,

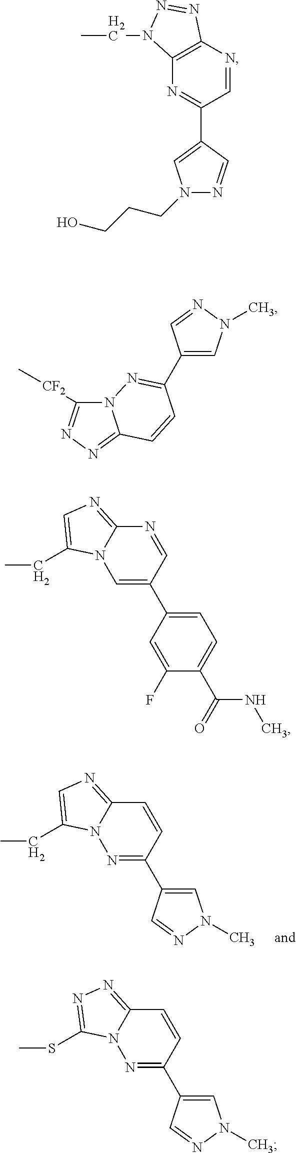

R22 is selected from hydrogen,

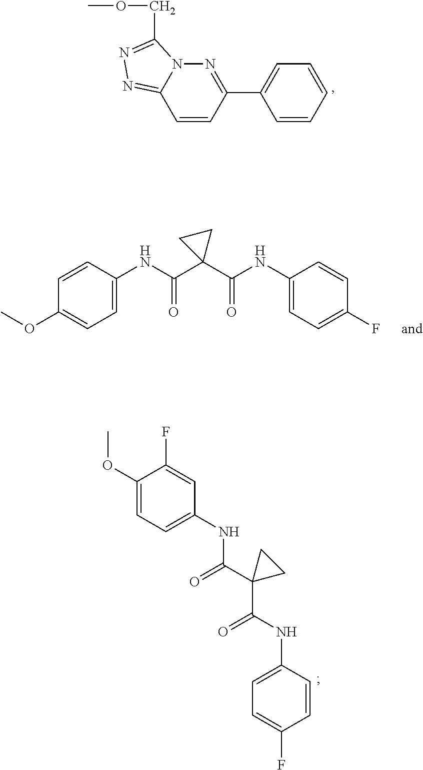 [SMILES: COCc1nnc2ccc(-c3ccccc3)nn12 ; COc1ccc(NC(=O)C2(C(=O)Nc3ccc(F)cc3)CC2)cc1 ; COc1ccc(NC(=O)C2(C(=O)Nc3ccc(F)cc3)CC2)cc1F]

and R23 is selected from hydrogen, hydroxyl, alkoxy and

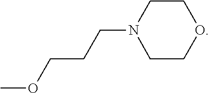

In some embodiments, the compound of formula V is:

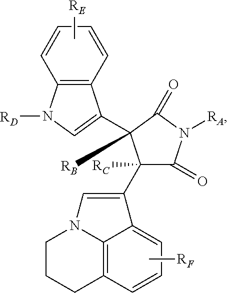

wherein each of R_{A}, R_{B}, R_{C}, R_{D}, R_{E}, and R_{F} is independently selected from hydrogen, C₁₋₆alkyl, C₁₋₆aryl, C₁₋₆alkoxy, halogen, hydroxyl and ammonium or any substituted form thereof.

In some embodiments, the compound of formula VI is:

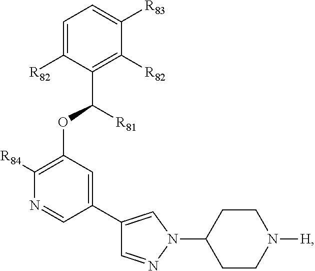

wherein, R81 is selected from a group comprising hydrogen, alkyl and alkoxy; R82 and R83 are independently halogen selected from chlorine, fluorine, bromine, iodine, and astatine; and R84 is selected from a group comprising amino, alkylamino.

In some embodiments, a cMet inhibitor is compound of formula V:

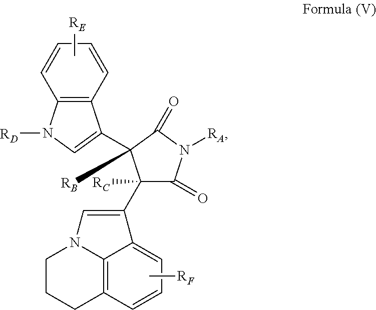

wherein each of R_{A}, R_{B}, R_{C}, R_{D}, R_{E}, and R_{F} is independently hydrogen.

In some embodiments, a cMet inhibitor is compound of formula VI

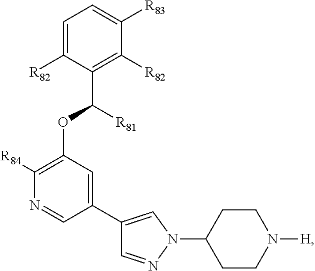

wherein, R81 is alkyl; R82 is chlorine; R83 is fluorine; and R84 is amino.

Non-limiting examples of CW231 include: HMG-CoA Reductase Inhibitors (CW231a): a) SIMVASTATIN or Zocor™ or [(1S,3R,7S,8S,8aR)-8-[2-[(2R,4R)-4-hydroxy-6-oxooxan-2-yl]ethyl]-3,7-dimethyl-1,2,3,7,8,8a-hexahydronaphthalen-1-yl]2,2-dimethylbutanoate or a pharmaceutically acceptable salt thereof; b) ATORVASTATIN Or Lipitor™ or Sotis, Torvast, Tozalip, Xavator, Atorvastatina, Atorvastatine, Atorvastatinium or 3R,5R)-7-[2-(4-fluorophenyl)-3-phenyl-4-(phenylcarbamoyl)-5-propan-2-ylpyrrol-1-yl]-3,5-dihydroxyheptanoic acid or a pharmaceutically acceptable salt thereof; c) CERIVASTATIN or Baycol™ or Cerivastatin sodium or Rivastatin or Lipobay, or Bay W6228; (E,3R,5S)-7-[4-(4-fluorophenyl)-5-(methoxymethyl)-2,6-di(propan-2-yl)pyridin-3-yl]-3,5-dihydroxyhept-6-enoate or a pharmaceutically acceptable salt thereof; d) FLUVASTATIN or Lescol™ or Fluindostatin or Cranoc (E,3R,5S)-7-[3-(4-fluorophenyl)-1-propan-2-ylindol-2-yl]-3,5-dihydroxyhept-6-enoic acid or a pharmaceutically acceptable salt thereof; e) LOVASTATIN or Mevacor™ or Altocor or Altoprev, Lovalip, Lovalord, Mevinacor, Mevinolin, Monacolin K, Nergadan [(1S,3R,7S,8S,8aR)-8-[2-[(2R,4R)-4-hydroxy-6-oxooxan-2-yl]ethyl]-3,7-dimethyl-1,2,3,7,8,8a-hexahydronaphthalen-1-yl](2S)-2-methylbutanoate or a pharmaceutically acceptable salt thereof; f) MEVASTATIN or Compactin or ML-236B or CS 500 [(1S,7S,8S,8aR)-8-[2-[(2R,4R)-4-hydroxy-6-oxooxan-2-yl]ethyl]-7-methyl-1,2,3,7,8,8a-hexahydronaphthalen-1-yl](2S)-2-methylbutanoate or a pharmaceutically acceptable salt thereof; g) PITAVASTATIN or NK-104 or AC1NR03J Or AKOS005145916 or Alpiza or Itavastatin or Itavastatin calcium or Livazo Or LS-74594 (E,3R,5S)-7-[2-cyclopropyl-4-(4-fluorophenyl)quinolin-3-yl]-3,5-dihydroxyhept-6-enoic acid or a pharmaceutically acceptable salt thereof; h) PRAVASTATIN or Pravachol™ or Eptastatin (3R,5R)-7-[(1S,2S,6S,8S,8aR)-6-hydroxy-2-methyl-8-[(2S)-2-methylbutanoyl]oxy-1,2,6,7,8,8a-hexahydronaphthalen-1-yl]-3,5-dihydroxyheptanoic acid or a pharmaceutically acceptable salt thereof; and i) ROSUVASTATIN or Crestor™ or (E,3R,5S)-7-[4-(4-fluorophenyl)-2-[methyl(methylsulfonyl)amino]-6-propan-2-ylpyrimidin-5-yl]-3,5-dihydroxyhept-6-enoic acid or a pharmaceutically acceptable salt thereof; ATP Citrate Lyase Inhibitors (CW231b): a) SB 204990 or (2R)-2-[(2S)-8-(2,4-dichlorophenyl)-2-hydroxyoctyl]-2-hydroxybutanedioic Acid or a pharmaceutically acceptable salt thereof; b) SB 201076 or (2S)-2-[(2S)-8-(2,4-dichlorophenyl)-2-hydroxyoctyl]-2-hydroxybutanedioic Acid or a pharmaceutically acceptable salt thereof; c) ETC-1002 Or ESP-55016 or ESP 55016 cr ETC-1002 cr 8-hydroxy-2,2,14,14-tetramethylpentadecanedioic acid cr a pharmaceutically acceptable salt thereof; and d) BMS-303141 or 3,5-Dichloro-2-hydroxy-N-(4-methoxy-[1,1′-biphenyl]-3-yl)-benzenesulfonamide or a pharmaceutically acceptable salt thereof; Farnesyltransferase Inhibitors (CW231c): a) Tipifamib or Zarnestra™ or R115777 or 6-[(R)-amino-(4-chlorophenyl)-(3-methylimidazol-4-yl)methyl]-4-(3-chlorophenyl)-1-methylquinolin-2-one or a pharmaceutically acceptable salt thereof; b) Lonafamib or Sarasar™ or SCH66336 or 4-[2-[4-[(11R)-3,10-dibromo-8-chloro-6,11-dihydro-5H-benzo[1, 2]cyclohepta[2,4-b]pyridin-11-yl]piperidin-1-yl]-2-oxoethyl]piperidine-1-carboxamide or a pharmaceutically acceptable salt thereof; c) BMS-214662 or (3R)-3-benzyl-1-(1H-imidazol-5-ylmethyl)-4-thiophen-2-ylsulfonyl-3,5-dihydro-2H-1,4-benzodiazepine-7-carbonitrile or a pharmaceutically acceptable salt thereof; d) L778123 or L-778123 or L-778,123 or ″4-[[5-[[4-(3-chlorophenyl)-3-oxopiperazin-1-yl]methyl]imidazol-1-yl]methyl]benzonitrile; hydrochloride or a pharmaceutically acceptable salt thereof; e) L-731734 or Nampamp-ile-hse lactone or 2-[[2-[(2-amino-3-sulfanylpropyl)amino]-3-methylpentyl]amino]-3-methyl-N-(2-oxooxolan-3-yl)pentanamide or a pharmaceutically acceptable salt thereof; f) B1086 or B956 or N-(4-methoxyanilino)-N-(4-methoxyphenyl)iminobenzenecarboximidamide or a pharmaceutically acceptable salt thereof; g) L-744832 or propan-2-yl (2S)-2-[[2-[(2S,3S)-2-[[(2R)-2-amino-3-sulfanylpropyl]amino]-3-methylpentoxy]-3-phenylpropanoyl]amino]-4-methylsulfonylbutanoate or a pharmaceutically acceptable salt thereof; h) BIM-46228 or a pharmaceutically acceptable salt thereof; i) FTI-276 or (2S)-2-[[4-[[(2R)-2-azaniumyl-3-sulfanylpropyl]amino]-2-phenylbenzoyl]amino]-4-methylsulfanylbutanoate or a pharmaceutically acceptable salt thereof; j) RPR-130401 or (3aRS,4SR,9SR,9aRS)-2-[2-(2-Methoxyphenyl)-2-propenoyl]-9-(4-methylphenyl)-2,3,3a,4,9,9a-hexahydro-1H-4,9-ethanobenzo[f]isoindole-3a-carboxylic acid or a pharmaceutically acceptable salt thereof; k) FTI-2148 or CTK8F9945 or AG-L-65695 or (2R)-2-[[4-[(1H-imidazol-5-ylmethylamino)methyl]-2-(2-methylphenyl)benzoyl]amino]-4-methylsulfanylbutanoic acid; 2,2,2-trifluoroacetic acid or a pharmaceutically acceptable salt thereof; 1) FTI-2628 or CTK8F9946 or AG-L-65696 or benzyl (2R)-2-[[4-[(1H-imidazol-5-ylmethylamino)methyl]-2-(2-methylphenyl)benzoyl]amino]-4-methylsulfanylbutanoate or a pharmaceutically acceptable salt thereof; m) FTI-277 or methyl (2S)-2-[[4-[[(2R)-2-amino-3-sulfanylpropyl]amino]-2-phenylbenzoyl]amino]-4-methylsulfanylbutanoate or a pharmaceutically acceptable salt thereof; n) FTase Inhibitor I or B581 or (2S)-2-[[(2S)-2-[[(2S)-2-[[(2R)-2-amino-3-sulfanylpropyl]amino]-3-methylbutyl]amino]-3-phenylpropanoyl]amino]-4-methylsulfanylbutanoic acid or a pharmaceutically acceptable salt thereof; o) FTase Inhibitor II or (2S)-2-[[4-[[(2R)-2-azaniumyl-3-sulfanylpropanoyl]amino]benzoyl]amino]-4-methylsulfanylbutanoate or a pharmaceutically acceptable salt thereof; p) FTase Inhibitor III or 2-[[2-[[2-[(2-amino-3-sulfanylpropanoyl)amino]-3-methylbutanoyl]amino]-3-naphthalen-2-ylpropanoyl]amino]-4-methylsulfanylbutanoic acid or a pharmaceutically acceptable salt thereof; q) L-745631 or [(3R)-4-[(2S)-2-amino-3-sulfanylpropyl]-3-(2-methoxyethyl)piperazin-1-yl]-naphthalen-1-ylmethanone or a pharmaceutically acceptable salt thereof; and r) L-739749 or methyl (2S)-2-[[(2S)-2-[(2S)-2-[[(2R)-2-amino-3-sulfanylpropyl]amino]-3-methylpentoxy]-3-phenylpropanoyl]amino]-4-methylsulfonylbutanoate or a pharmaceutically acceptable salt thereof; Geranylgeranyltransferase Inhibitors (CW231d): a) GGTI-2418 Or (S)-2-((S)-2-benzyl-4-((4-methyl-1H-imidazol-5-yl)methyl)-3-oxopiperazine-1-carboxamido)-4-methylpentanoic acid or a pharmaceutically acceptable salt thereof; b) GGTI-2133 or (2S)-2-[[4-(1H-imidazol-5-ylmethylamino)-2-naphthalen-1-ylbenzoyl]amino]-4-methylpentanoic acid; 2,2,2-trifluoroacetic acid or a pharmaceutically acceptable salt thereof; c) GGTI-2147 or methyl (2S)-2-[[4-(1H-imidazol-5-ylmethylamino)-2-naphthalen-1-ylbenzoyl]amino]-4-methylpentanoate or a pharmaceutically acceptable salt thereof; d) GGTI-2154 or a pharmaceutically acceptable salt thereof; e) GGTI-2166 or a pharmaceutically acceptable salt thereof; f) GGTI-286 or methyl (2S)-2-[[4-[[(2R)-2-amino-3-sulfanylpropyl]amino]-2-phenylbenzoyl]amino]-4-methylpentanoate or a pharmaceutically acceptable salt thereof; g) GGTI-287 or (2S)-2-[[4-[[(2R)-2-azaniumyl-3-sulfanylpropyl]amino]-2-phenylbenzoyl]amino]-4-methylpentanoate or a pharmaceutically acceptable salt thereof; h) GGTI-297 or (2S)-2-[[4-[[(2R)-2-azaniumyl-3-sulfanylpropyl]amino]-2-naphthalen-1-ylbenzoyl]amino]-4-methylpentanoate or a pharmaceutically acceptable salt thereof; and i) GGTI-298 or methyl (2S)-2-[[4-[[(2R)-2-amino-3-sulfanylpropyl]amino]-2-naphthalen-1-ylbenzoyl]amino]-4-methylpentanoate or a pharmaceutically acceptable salt thereof; and Farnesyl diphosphate synthatase inhibitors (CW231e): a) Zoledronate or Zoledronic acid or Zometa™ or Reclast™ or Aclasta™ or (1-hydroxy-2-imidazol-1-yl-1-phosphonoethyl)phosphonic acid or a pharmaceutically acceptable salt thereof; b) Risedronate or Risedronic acid or 105462-24-6 or Benet™ or Acido risedronico or Bisphosphonate 1 or (1-hydroxy-1-phosphono-2-pyridin-3-ylethyl)phosphonic acid or a pharmaceutically acceptable salt thereof; c) Ibandronate or Bondronat™ or Bonviva™ or Boniva™ or ibandronic acid or [1-hydroxy-3-[methyl(pentyl)amino]-1-phosphonopropyl]phosphonic acid or a pharmaceutically acceptable salt thereof; d) Alendronate or Alendronic acid or Fosamax™ or Arendal or (4-amino-1-hydroxy-1-phosphonobutyl)phosphonic acid or a pharmaceutically acceptable salt thereof; e) Pamidronate or Pamidronic acid or amidronate or Aredia™ or Aminomux™ or (3-amino-1-hydroxy-1-phosphonopropyl)phosphonic acid or a pharmaceutically acceptable salt thereof; f) Etidronate or Etidronic Acid or HEDP or EHDP or Etidronsaeure or Turpinal SL™ or Acetodiphosphonic acid or (1-hydroxy-1-phosphonoethyl)phosphonic acid or a pharmaceutically acceptable salt thereof; g) Clodronate or Clodronic Acid or Dichloromethanediphosphonic acid or Bonefos™ or Dichloromethylidene diphosphonate or Clodronsaeure or [dichloro(phosphono)methyl]phosphonic acid or a pharmaceutically acceptable salt thereof; h) Neridronate or Neridronic acid or (6-amino-1-hydroxy-1-phosphonohexyl)phosphonic acid or a pharmaceutically acceptable salt thereof; i) Tiludronate or Tiludronic acid or [(4-chlorophenyl)sulfanyl-phosphonomethyl]phosphonic acid or a pharmaceutically acceptable salt thereof; j) Incadronate or Cimadronate or Bisphonal™ or Cimadronate sodium or disodium; [(cycloheptylamino)-[hydroxy(oxido)phosphoryl]methyl]-hydroxyphosphinate or a pharmaceutically acceptable salt thereof; k) Olpadronate or Olpadronic acid or AHGA diphosphonate or dimethyl-pamidronate or [3-(dimethylamino)-1-hydroxy-1-phosphonopropyl]phosphonic acid or a pharmaceutically acceptable salt thereof; 1) EB-1053 or disodium; (1-hydroxy-1-phosphonato-3-pyrrolidin-1-ylpropyl)phosphonic acid or a pharmaceutically acceptable salt thereof; and m) Minodronate or Minodronic acid or Minodronate or Bonoteo™ or Recalbon™ or YM-529 or (1-hydroxy-2-imidazo[1,2-a]pyridin-3-yl-1-phosphonoethyl)phosphonic acid or a pharmaceutically acceptable salt thereof.

Non-limiting examples of CW231 include the following:

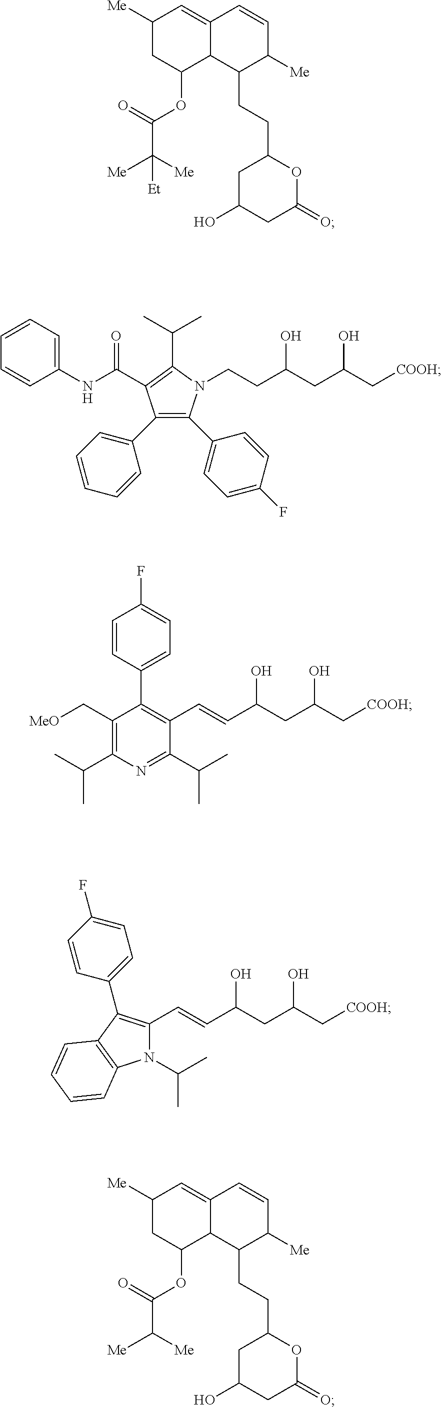 [SMILES: CCC(C)(C)C(=O)OC1CC(C)C=C2C=CC(C)C(CCC3CC(O)CC(=O)O3)C21 ; CC(C)c1c(C(=O)Nc2ccccc2)c(-c2ccccc2)c(-c2ccc(F)cc2)n1CCC(O)CC(O)CC(=O)O ; COCc1c(C(C)C)nc(C(C)C)c(/C=C/C(O)CC(O)CC(=O)O)c1-c1ccc(F)cc1 ; CC(C)n1c(/C=C/C(O)CC(O)CC(=O)O)c(-c2ccc(F)cc2)c2ccccc21 ; CC1C=C2C=CC(C)C(CCC3CC(O)CC(=O)O3)C2C(OC(=O)C(C)C)C1]

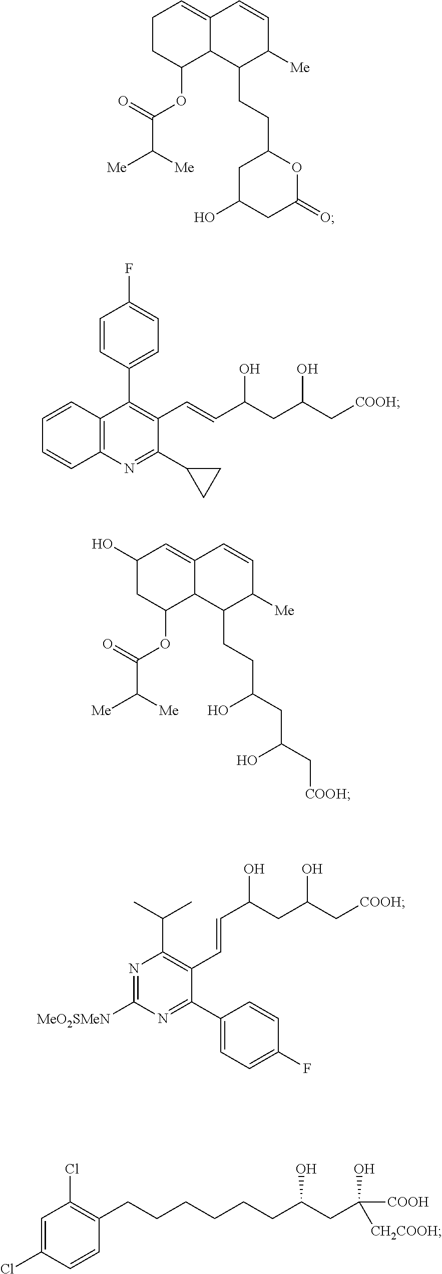

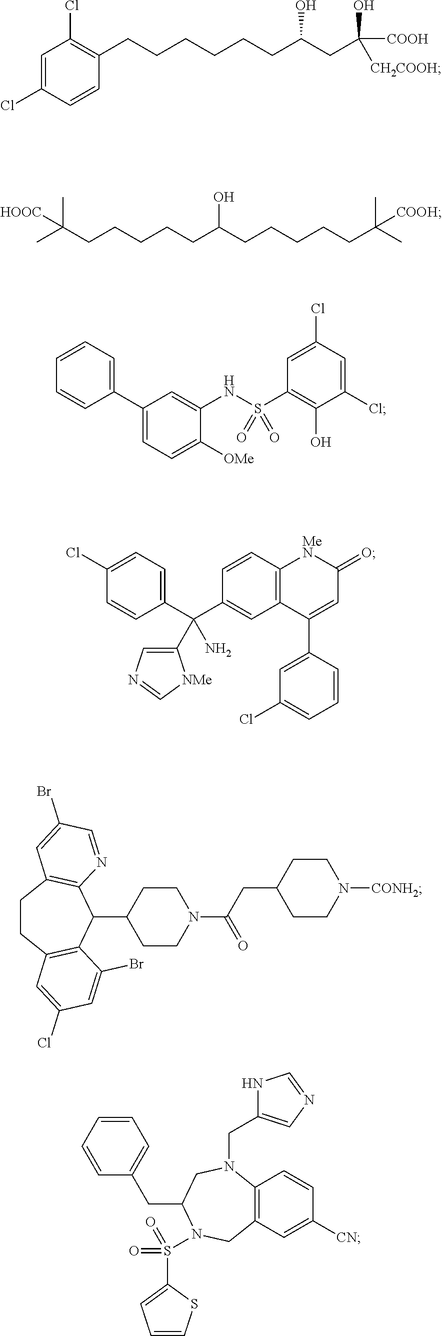

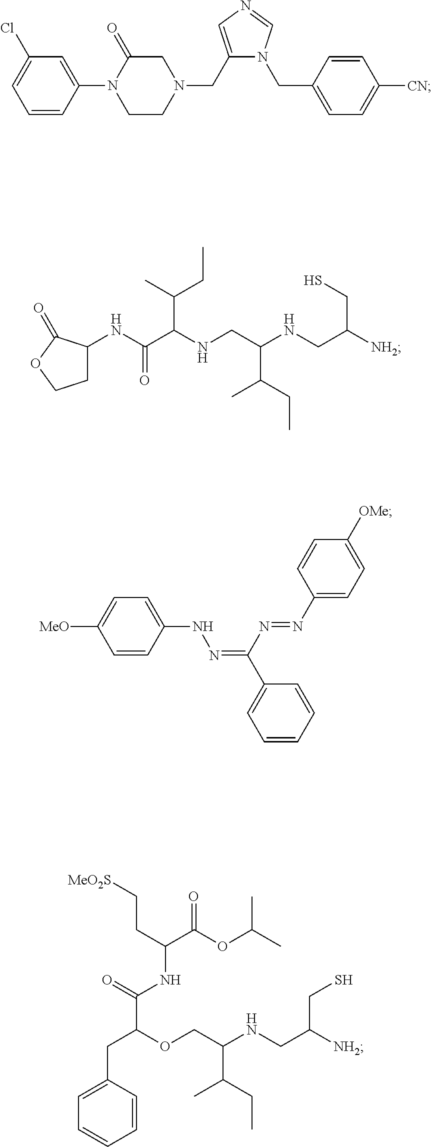 [SMILES: N#Cc1ccc(Cn2cncc2CN2CCN(c3cccc(Cl)c3)C(=O)C2)cc1 ; CCC(C)C(CNC(C(=O)NC1CCOC1=O)C(C)CC)NCC(N)CS ; COc1ccc(/N=N/C(=N\Nc2ccc(OC)cc2)c2ccccc2)cc1 ; CCC(C)C(COC(Cc1ccccc1)C(=O)NC(CCS(C)(=O)=O)C(=O)OC(C)C)NCC(N)CS]

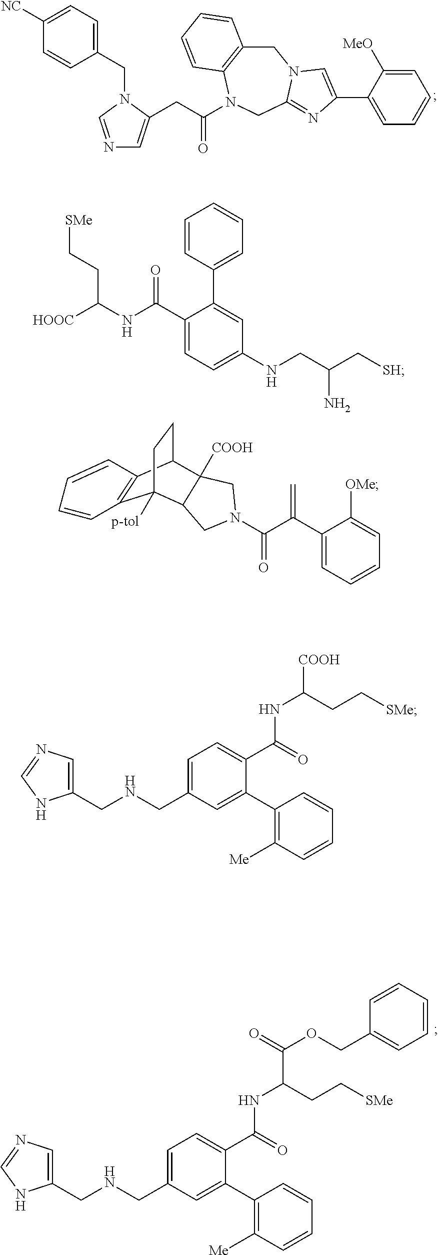

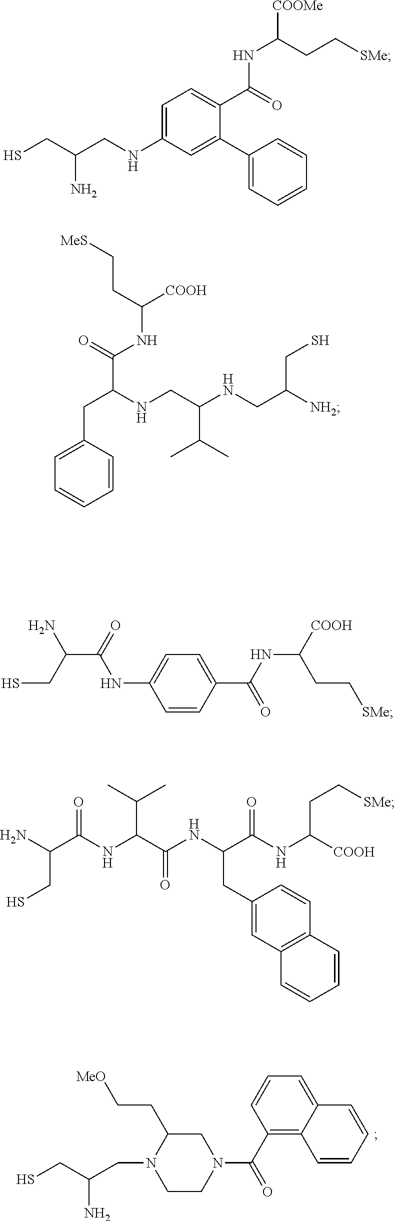 [SMILES: COC(=O)C(CCSC)NC(=O)c1ccc(NCC(N)CS)cc1-c1ccccc1 ; CSCCC(NC(=O)C(Cc1ccccc1)NCC(NCC(N)CS)C(C)C)C(=O)O ; CSCCC(NC(=O)c1ccc(NC(=O)C(N)CS)cc1)C(=O)O ; CSCCC(NC(=O)C(Cc1ccc2ccccc2c1)NC(=O)C(NC(=O)C(N)CS)C(C)C)C(=O)O ; COCCC1CN(C(=O)c2cccc3ccccc23)CCN1CC(N)CS]

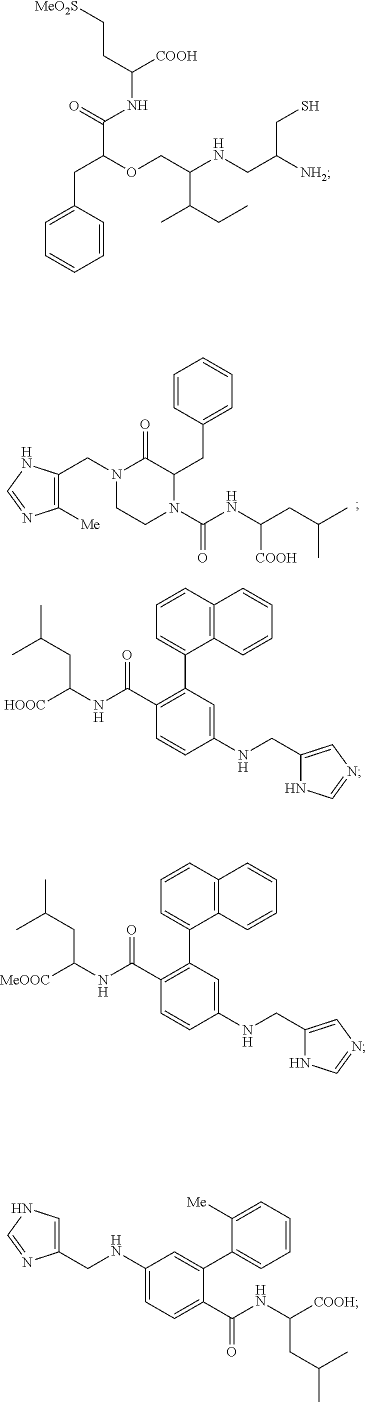

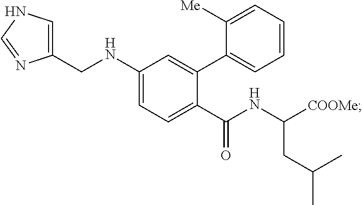 [SMILES: COC(=O)C(CC(C)C)NC(=O)c1ccc(NCc2c[nH]cn2)cc1-c1ccccc1C]

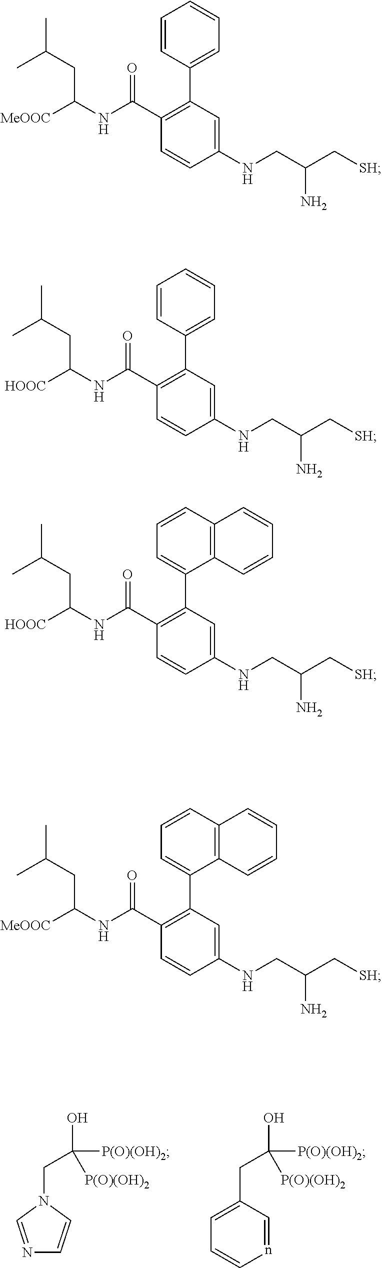

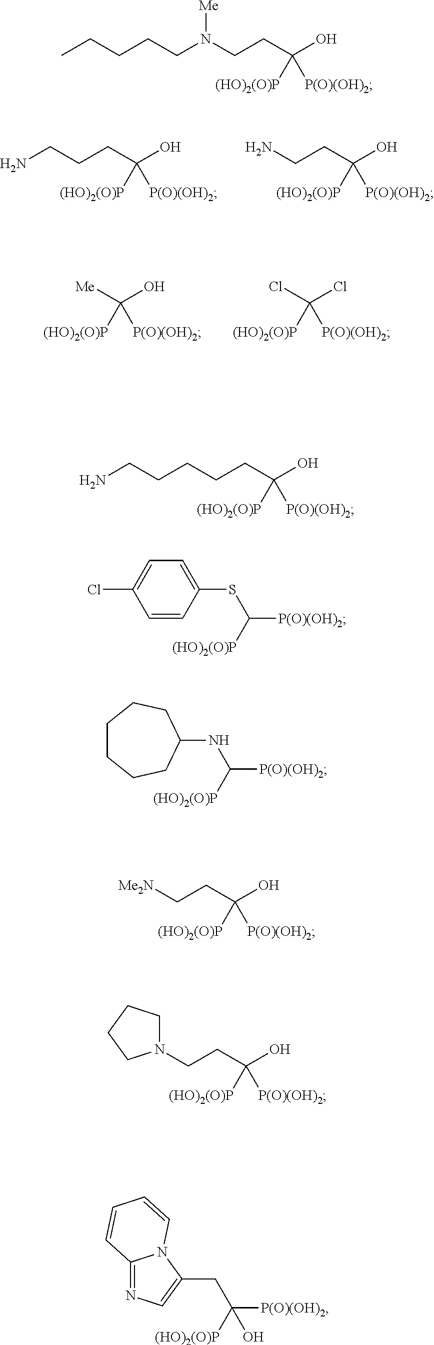

and pharmaceutically-acceptable salts thereof.

In some embodiments, CW231a is HMG CoA reductase inhibitor represented by a compound of formula VII or VIII.

In some embodiments, the compound of formula VII is:

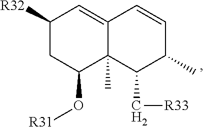

wherein, R31 is selected from

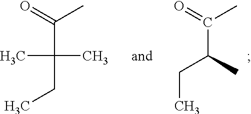

R32 is selected from hydrogen, alkyl and hydroxy; and R33 is selected from

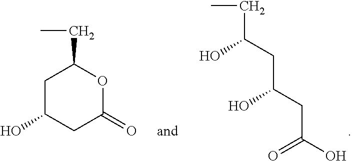

In some embodiments, the compound of formula VIII is

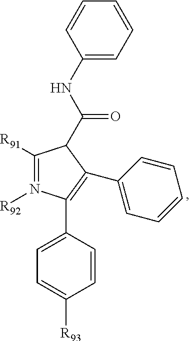

wherein R91 is selected from methyl, ethyl, propyl, isopropyl, butyl, and isobutyl; R92 is

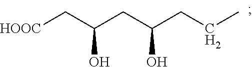

and R93 is selected from fluorine, chlorine, bromine, iodine, and astatine.

In some embodiments, an HMG CoA reductase inhibitor is compound of formula VII

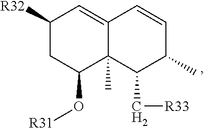

wherein, R31 is

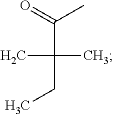

R32 is alkyl; and R33 is

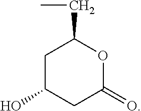

In some embodiments, HMG CoA reductase inhibitor is compound of formula VIII

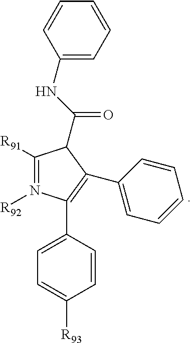

wherein R91 is ethyl; R92 is

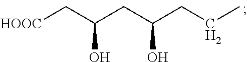

and R93 is fluorine.

In some embodiments, CW231b is ATP citrate lysase inhibitor represented by a compound of formula IX or X.

In some embodiments, the compound of formula IX is

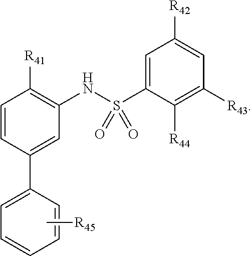

wherein, R41 is selected from C₁₋₆alkyl, C₁₋₆alkoxy, hydroxyl and hydrogen; each of R42 and R43 is independently selected from hydrogen, halogen, hydroxy, C₁₋₆alkyl and C₁₋₆alkoxy; R44 is selected from hydrogen, halogen, C₁₋₆alkyl, C₁₋₆alkoxy, and hydroxy; and R45 is selected from hydrogen, halogen, C₁₋₆alkyl, C₁₋₆alkoxy and hydroxy.

In some embodiments, the compound of formula X is

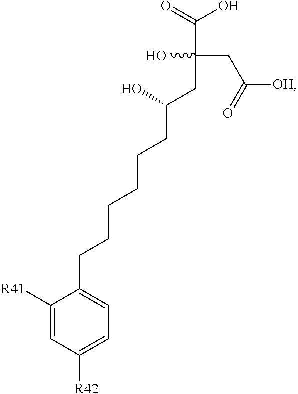

wherein, each of R41 and R42 is independently selected from hydrogen and halogen.

In some embodiments, an ATP citrate lyase inhibitor is compound of formula IX:

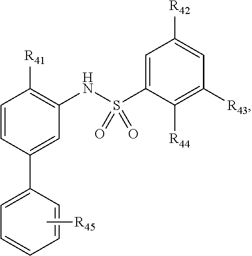

wherein, R41 is methoxy; each of R42 and R43 is independently halogen, selected from fluorine, chlorine, bromine, idodine, and astatine; and R44 is hydroxy.

In some embodiments, CW231c is a farnesyl transferase inhibitor represented by a compound of formula XI or XII.

In some embodiments, the compound of formula XI is:

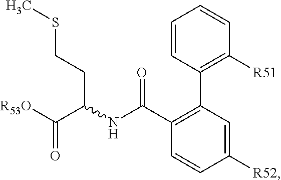

wherein, R51 is hydrogen or alkyl; R52 is selected from

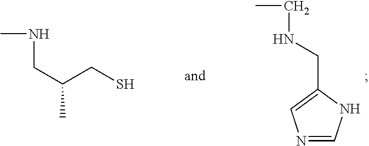

and R53 is selected from hydrogen, benzyl and alkyl.

In some embodiments, the compound of formula XII is:

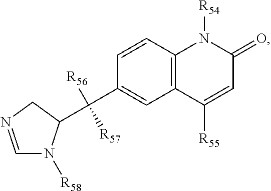

wherein, R54 is selected from hydrogen, C1-6alkyl, C1-6alkoxy and hydroxy; R55 is selected from hydrogen, C1-6alkyl and halobenzene; R56 is selected from hydrogen, C1-6alkyl and halobenzene; R57 is selected from hydrogen, hydroxyl and amino; and R58 is selected from hydrogen, C1-6alkyl, C1-6alkoxy and hydroxy.

In some embodiments, a farnesyl transferase inhibitor is compound of formula XII wherein, R54 is C1-6alkyl; each of R55 and R56 is independently halophenyl; R57 is amino group; and R58 is C1-6alkyl, as provided below:

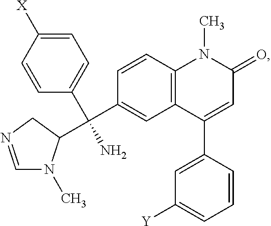

wherein, each of X and Y is independently selected from fluorine, chlorine, bromine, iodine, and astatine.

In some embodiments, CW231d is geranyl geranyl transferase inhibitor represented by compound of formula XIII or XIV.

In some embodiments, the compound of formula XIII is:

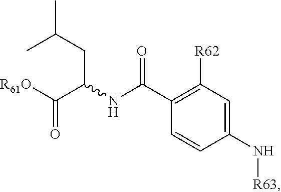

wherein, R61 is hydrogen or alkyl; R62 is selected from:

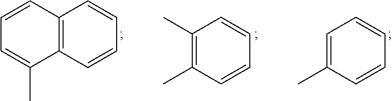

and R63 is selected from

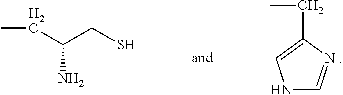

In some embodiments, the compound of formula XIV is:

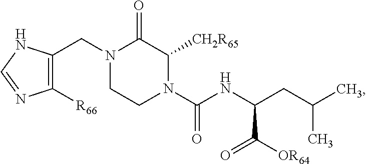

wherein, R64 is selected from hydrogen and C₁₋₆alkyl; R65 is selected from:

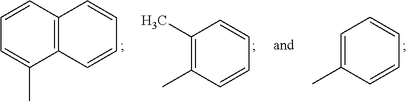

and R66 is selected from hydrogen, hydroxy, C₁₋₆alkyl, C₁₋₆alkoxy, and halogen.

In some embodiments, a geranyl geranyl transferase inhibitor is compound of formula XIV wherein, R64 is hydrogen; R65 is

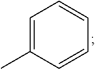

and R66 is C₁₋₆alkyl, as provided below:

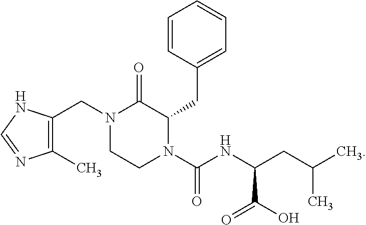 [SMILES: Cc1nc[nH]c1CN1CCN(C(=O)N[C@@H](CC(C)C)C(=O)O)[C@@H](Cc2ccccc2)C1=O]

In some embodiments, CW231e is a farnesyl diphosphate synthatase inhibitor represented by a compound of formula XV:

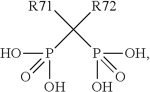

wherein, R71 is selected from hydrogen, hydroxyl, and halogen; and R72 is selected from alkyl, halogen,

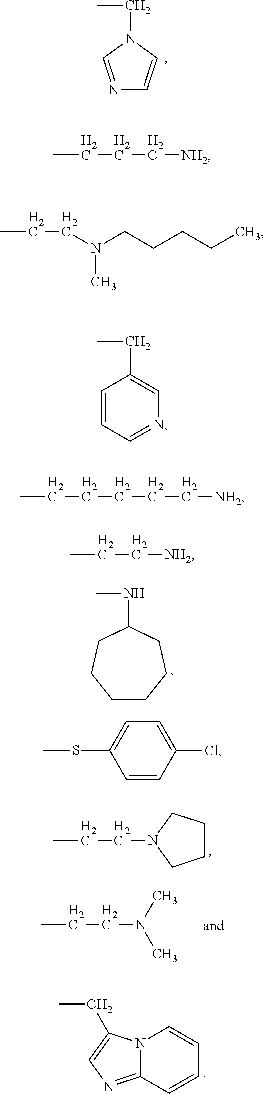

In some embodiments, a farnesyl diphosphate synthatase inhibitor is a compound of formula XV: wherein R71 is hydroxyl; and R72 is

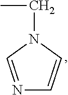

as provided below:

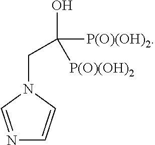

In some embodiments, a compound used herein is of the formula:

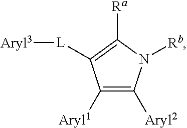

wherein
Aryl¹ is an aryl group or a heteroaryl group, any of which is substituted or unsubstituted;
Aryl² is an aryl group or a heteroaryl group, any of which is substituted or unsubstituted;
Aryl³ is an aryl group or a heteroaryl group, any of which is substituted or unsubstituted;
L is an alkylene group, an alkenylene group, an alkynylene group, an ether linkage, an ester linkage, an amide linkage, a carbamate linkage, a carbonate linkage, a urethane linkage, a sulfone linkage, a thioether linkage, or a thioester linkage, any of which is substituted or unsubstituted, or a chemical bond;
R^{a} is H, an alkyl group, an alkenyl group, an alkynyl group, an aryl group, and aralkyl group, a heterocyclyl group, a heterocyclylalkyl group, a heteroaryl group, a heterocyclylalkyl group, an alkoxy group, or a halogen, any of which is substituted or unsubstituted; and
R^{b} is H, an alkyl group, an alkenyl group, an alkynyl group, an aryl group, and aralkyl group, a heterocyclyl group, a heterocyclylalkyl group, a heteroaryl group, or a heterocyclylalkyl group, any of which is substituted or unsubstituted,
   or a pharmaceutically-acceptable salt thereof.

In some embodiments: Aryl¹ is an aryl group, which is substituted or unsubstituted; Aryl² is an aryl group, which is substituted or unsubstituted; Aryl³ is an aryl group, which is substituted or unsubstituted; L is an ether linkage, an ester linkage, an amide linkage, a carbamate linkage, a carbonate linkage, or a urethane linkage, any of which is substituted or unsubstituted; R^{a} is an alkyl group, which is substituted or unsubstituted; and R^{b} is an alkyl group, which is substituted or unsubstituted.

In some embodiments, the compound is:

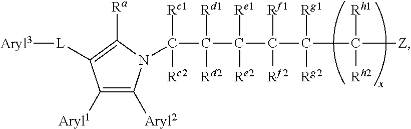

wherein:
x is 0, 1, 2, 3, 4, 5, 6, 7, 8, 9, or 10;
each of R^{c1}, R^{c2}, R^{d1}, R^{d2}, R^{e1}, R^{e2}, R^{f1}, R^{f2}, R^{g1}, R^{g2}, R^{h1}, and R^{h2} is independently H, OH, SH, alkyl, alkenyl, alkynyl, alkoxy, amino, an ester, an amide, a carboxylic acid, nitro, halo, or cyano; and
Z is H, OH, SH, a carboxylic acid, an ester, or an amide, or
any of Z, R^{c1}, R^{c2}, R^{d1}, R^{d2}, R^{e1}, R^{e2}, R^{f1}, R^{f2}, R^{g1}, R^{g2}, R^{h1}, and R^{h2} together form a ring that is substituted or unsubstituted.

In some embodiments, Aryl¹ is a phenyl group, which is substituted or unsubstituted; Aryl² is a phenyl group, which is substituted or unsubstituted; and Aryl³ is a phenyl group, which is substituted or unsubstituted. In some embodiments, Aryl¹ is a phenyl group; Aryl² is a phenyl group, which is para-substituted with a halogen; and Aryl³ is a phenyl group. The halogen can be F, Br, Cl, or Br. In some embodiments, the halogen is F.

In some embodiments, L is an amide linkage.

In some embodiments, x is 1.

In some embodiments, the compound is:

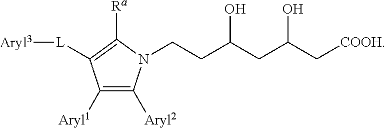

In some embodiments, the compound is:

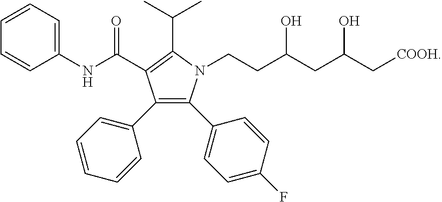 [SMILES: CC(C)c1c(C(=O)Nc2ccccc2)c(-c2ccccc2)c(-c2ccc(F)cc2)n1CCC(O)CC(O)CC(=O)O]

In some embodiments, the compound is:

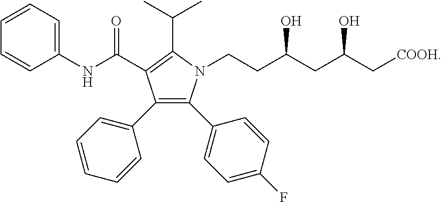 [SMILES: CC(C)c1c(C(=O)Nc2ccccc2)c(-c2ccccc2)c(-c2ccc(F)cc2)n1CC[C@@H](O)C[C@@H](O)CC(=O)O]

In some embodiments, a compound used herein is of the formula:

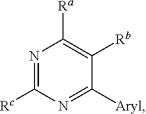

wherein:
Aryl is an aryl group or a heteroaryl group, any of which is substituted or unsubstituted;
R^{a} is H, an alkyl group, an alkenyl group, an alkynyl group, an aryl group, and aralkyl group, a heterocyclyl group, a heterocyclylalkyl group, a heteroaryl group, a heterocyclylalkyl group, an alkoxy group, or a halogen, any of which is substituted or unsubstituted;
R^{b} is H, an alkyl group, an alkenyl group, an alkynyl group, an aryl group, and aralkyl group, a heterocyclyl group, a heterocyclylalkyl group, a heteroaryl group, or a heterocyclylalkyl group, any of which is substituted or unsubstituted;
R^{c} is H, an amino group, an amido group, a sulfonamide, a sulfonic acid, or a sulfamic acid,
   or a pharmaceutically-acceptable salt thereof.

In some embodiments: Aryl is an aryl group, which is substituted or unsubstituted; R^{a} is an alkyl group, which is substituted or unsubstituted; and R^{b} is an alkeneyl group, which is substituted or unsubstituted.

In some embodiments, the compound is:

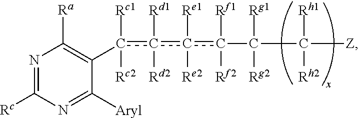

wherein
x is 0, 1, 2, 3, 4, 5, 6, 7, 8, 9, or 10;
each [symbol] is independently a single or double bond of E or Z configuration;
each of R^{c1}, R^{c2}, R^{d1}, R^{d2}, R^{e1}, R^{e2}, R^{f1}, R^{f2}, R^{g1}, R^{g2}, R^{h1}, and R^{h2} is independently H, OH, SH, alkyl, alkenyl, alkynyl, alkoxy, amino, an ester, an amide, a carboxylic acid, nitro, halo, cyano, or absent; and
Z is H, OH, SH, a carboxylic acid, an ester, or an amide, or
any of Z, R^{c1}, R^{c2}, R^{d1}, R^{d2}, R^{e1}, R^{e2}, R^{f1}, R^{f2}, R^{g1}, R^{g2}, R^{h1}, and R^{h2} together form a ring that is substituted or unsubstituted.

In some embodiments, the compound is:

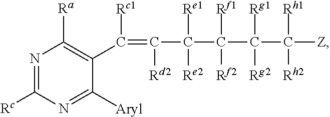

wherein the double bond is of the E configuration.

In some embodiments, Aryl is a phenyl group, which is substituted or unsubstituted. In some embodiments, Aryl is a phenyl group, which is para-substituted with a halogen. The halogen can be F, Br, Cl, or Br. In some embodiments, the halogen is F.

In some embodiments, x is 1.

In some embodiments, R^{c} is a sulfonamide.

In some embodiments, the compound is:

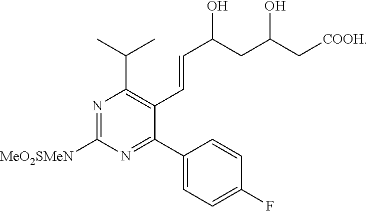

In some embodiments, the compound is:

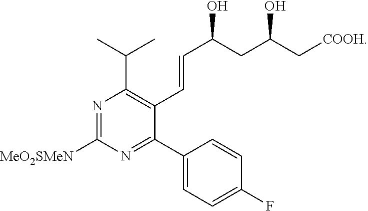

In some embodiments, a compound used herein is of the formula:

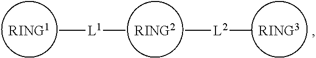

wherein:
L¹ is alkylene, alkeneylene, alkynylene, a cyclic group, a heterocyclic group, an ether linkage, an ester linkage, an amide linkage, a carbamate linkage, a carbonate linkage, a urethane linkage, a thioether linkage, or a thioester linkage, any of which is substituted or unsubstituted, or

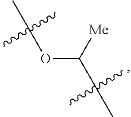 [SMILES: *OC(*)C]

or a chemical bond;
L² is alkylene, alkeneylene, alkynylene, a cyclic group, a heterocyclic group, an ether linkage, an ester linkage, an amide linkage, a carbamate linkage, a carbonate linkage, a urethane linkage, a thioether linkage, or a thioester linkage, any of which is substituted or unsubstituted, or

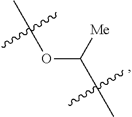 [SMILES: *OC(*)C]

or a chemical bond;
RING¹ is:

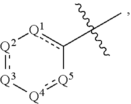

wherein:
   Q¹ is N or CR^{j1}; Q² is N or CR^{j2}; Q³ is N or CR^{j3}; Q⁴ is N or CR^{j4}; Q⁵ is N or CR^{j5};
   each [symbol] is independently saturated or unsaturated; and
   each of R^{j1}, R^{j2}, R^{j3}, R^{j4}, and R^{j5} is independently H, halogen, alkyl, OH, SH, an amino group, an amido group, an ester group, or a carbamate group, any of which is substituted or unsubstituted, or is absent;
RING² is:

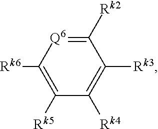

wherein:
   Q⁶ is N or CR^{k1};
   R^{k1} is H, halogen, alkyl, OH, SH, an amino group, an amido group, an ester group, or a carbamate group, any of which is substituted or unsubstituted, a connection to L¹, a connection to L², or is absent; and
   each of R^{k2}, R^{k3}, R^{k4}, R^{k5}, and R^{k6} is independently H, halogen, alkyl, OH, SH, an amino group, an amido group, an ester group, or a carbamate group, any of which is substituted or unsubstituted, a connection to L¹, or a connection to L²; and
RING³ is:

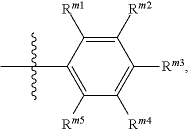

wherein:
   each of R^{m1}, R^{m2}, R^{m3}, R^{m4}, and R^{m5} is independently H, halogen, alkyl, OH, SH, an amino group, an amido group, an ester group, or a carbamate group, any of which is substituted or unsubstituted,
      or a pharmaceutically-acceptable salt thereof.

In some embodiments, each of R^{m1}, R^{m2}, R^{m3}, R^{m4}, and R^{m5} is independently H, halogen, or alkyl that is substituted with halogen. In some embodiments, each of R^{m1}, R^{m2}, R^{m3}, R^{m4}, and R^{m5} is independently H, F, Cl, or CF₃.

In some embodiments, R^{j2} is an amido group.

In some embodiments, RING¹ is:

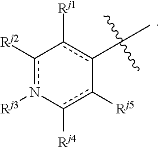

In some embodiments, RING¹ is:

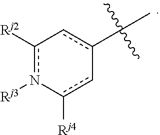

In some embodiments, RING¹ is:

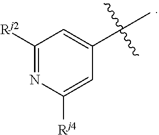

In some embodiments, RING¹ is:

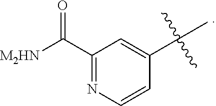

In some embodiments, RING¹ is:

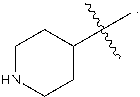 [SMILES: *C1CCNCC1]

In some embodiments, RING² is:

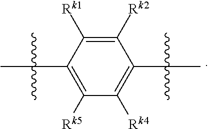

In some embodiments, RING² is:

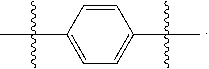 [SMILES: *c1ccc(*)cc1]

In some embodiments, RING² is:

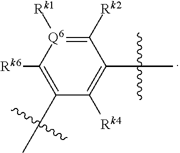

In some embodiments, RING² is:

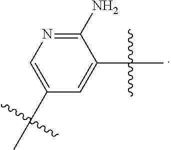 [SMILES: *c1cnc(N)c(*)c1]

In some embodiments, RING³ is:

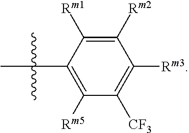

In some embodiments, RING³ is:

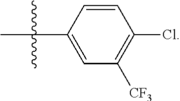 [SMILES: *c1ccc(Cl)c(C(F)(F)F)c1]

In some embodiments, RING³ is:

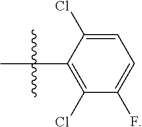 [SMILES: *c1c(Cl)ccc(F)c1Cl]

In some embodiments, L¹ is alkylene, a cyclic group, a heterocyclic group, an ether linkage, an ester linkage, an amide linkage, a carbamate linkage, a urethane linkage, any of which is substituted or unsubstituted, or

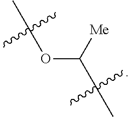 [SMILES: *OC(*)C]

In some embodiments, L² is alkylene, a cyclic group, a heterocyclic group, an ether linkage, an ester linkage, an amide linkage, a carbamate linkage, a urethane linkage, any of which is substituted or unsubstituted, or

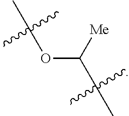 [SMILES: *OC(*)C]

In some embodiments, L¹ is a heterocyclic group, an ether linkage, a urethane linkage, any of which is substituted or unsubstituted, or

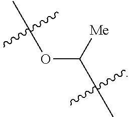 [SMILES: *OC(*)C]

In some embodiments, L² is a heterocyclic group, an ether linkage, a urethane linkage, any of which is substituted or unsubstituted, or

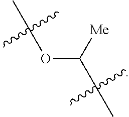 [SMILES: *OC(*)C]

In some embodiments, L¹ is an ether linkage.

In some embodiments, L¹ is

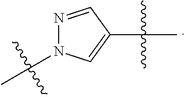 [SMILES: *c1cnn(*)c1]

In some embodiments, L² is a urethane linkage

In some embodiments, L² is

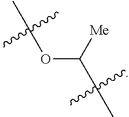 [SMILES: *OC(*)C]

In some embodiments, L² is

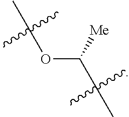 [SMILES: *O[C@@H](*)C]

In some embodiments, the compound is:

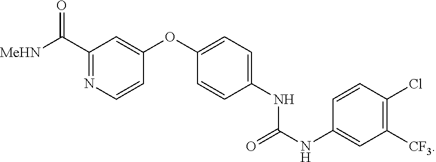 [SMILES: CNC(=O)c1cc(Oc2ccc(NC(=O)Nc3ccc(Cl)c(C(F)(F)F)c3)cc2)ccn1]

In some embodiments, the compound is:

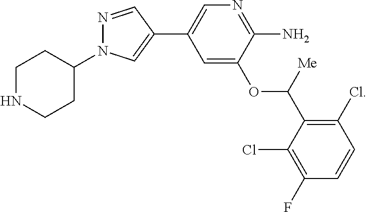 [SMILES: CC(Oc1cc(-c2cnn(C3CCNCC3)c2)cnc1N)c1c(Cl)ccc(F)c1Cl]

In some embodiments, the compound is:

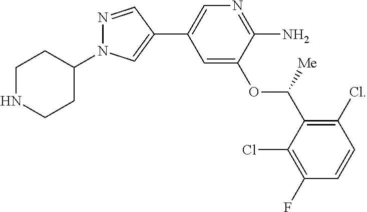 [SMILES: C[C@@H](Oc1cc(-c2cnn(C3CCNCC3)c2)cnc1N)c1c(Cl)ccc(F)c1Cl]

### Pharmaceutically-Acceptable Salts.

The disclosure further provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal.

In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, a iron salt, a calcium salt, a strontium salt, a cobalt salt, a titanium salt, an aluminum salt, a copper salt, a cadmium salt, or a zinc salt. Ammonium salts can arise from the addition of ammonia or an organic amine to a compound of the disclosure.

In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the disclosure.

In some embodiments, the acid is organic.

In some embodiments, the acid is inorganic.

In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Pharmaceutical Compositions.

The present disclosure further relates to a process for obtaining a composition of a compound of CW137 and a compound of CW231, optionally along with pharmaceutically acceptable excipient(s), the process comprising combining the compounds in any order thereof.

The present disclosure also further relates to a process for obtaining a composition of a compound of CW147 and a compound of CW231, optionally along with pharmaceutically acceptable excipient(s), the process comprising combining the compounds in any order thereof.

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by any form and route known in the art including, for example, intravenous, subcutaneous, intramuscular, oral, rectal, aerosol, parenteral, ophthalmic, pulmonary, transdermal, vaginal, otic, nasal, and topical administration.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the active compounds with pharmaceutically acceptable carriers or excipients well known in the art. Such carriers can be used to formulate tablets, powders, pills, dragees, capsules, liquids, gels, syrups, elixirs, slurries, suspensions and the like, for oral ingestion by a subject.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which may optionally contain an excipient such as gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline processed. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parental injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

Formulations suitable for transdermal administration of the active compounds can employ transdermal delivery devices and transdermal delivery patches, and can be lipophilic emulsions or buffered aqueous solutions, dissolved and/or dispersed in a polymer or an adhesive. Such patches may be constructed for continuous, pulsatile, or on demand delivery of pharmaceutical compounds. Transdermal delivery can be accomplished by means of iontophoretic patches and the like. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and means to secure the device to the skin.

For administration by inhalation, the active compounds can be in a form as an aerosol, a mist, or a powder. Pharmaceutical compositions are conveniently delivered in the form of an aerosol spray presentation from pressurized packs or a nebuliser, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compounds and a suitable powder base such as lactose or starch.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, is first melted.

In practicing the methods of treatment or use provided herein, therapeutically-effective amounts of the compounds described herein are administered in pharmaceutical compositions to a subject having a disease or condition to be treated.

In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compounds described herein can be manufactured in a conventional manner, for example, by means of conventional mixing, dissolving, granulating, dragee-making, levigating, emulsifying, encapsulating, entrapping or compression processes.

The pharmaceutical compositions can include at least one pharmaceutically acceptable carrier, diluent, or excipient and compounds described herein as free-base or pharmaceutically-acceptable salt form. The methods and pharmaceutical compositions described herein include the use crystalline forms (also known as polymorphs), and active metabolites of these compounds having the same type of activity.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellarvesicle (SUV), and the large unilamellar vesicle (LUV). Liposomal physicochemical properties can be modulated to optimize penetration through biological barriers and retention at the site of administration, and to prevent premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Compounds can be delivered via antibody-drug conjugates (ADCs) technology. Here, drugs are conjugated/fused to tumor-specific antibodies so as to deliver the drug to the site of tumor and increase their therapeutic efficacy. ADCs have been developed for targeted delivery of anti-cancer drugs to tumor in the patient body with the aim of bypassing the morbidity common to conventional drug delivery.

Non-limiting examples of dosage forms suitable for use in the disclosure include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, plant cellulosic material and spheronization agents, and any combination thereof.

A composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, granular masses, and the like.

Compositions of the disclosure can be delivered via a time-controlled delivery system. An example of a suitable time-controlled delivery system is the PULSINCAP® system, or a variant thereof. The time-controlled delivery system can further comprise pH-dependent systems, microbially-triggered delivery systems, or a combination thereof. The time-controlled system may comprise a water insoluble capsule body enclosing a drug reservoir. The capsule body can be closed at one end with a hydrogel plug. The hydrogel plug can comprise swellable polymers, erodible compressed polymers, congealed melted polymers, enzymatically-controlled erodible polymers, or a combination thereof. The swellable polymers can include polymethacrylates. Non-limiting examples of erodible compressed polymers include hydroxypropyl methylcellulose, polyvinyl alcohol, polyvinyl acetate, polyethylene oxide, and combinations thereof. Non-limiting examples of congealed melted polymers include saturated polyglycolated glycerides, glycerylmonooleate, and combinations thereof. Non-limiting examples of enzymatically-controlled erodible polymers include polysaccharides; amylose; guar gum; pectin; chitosan; inulin; cyclodextrin; chondroitin sulphate; dextrans; locust bean gum; arabinogalactan; chondroitin sulfate; xylan; calcium pectinate; pectin/chitosan mixtures; amidated pectin; and combinations thereof.

The time-controlled delivery system can comprise a capsule, which further comprises an organic acid. The organic acid can be filled into the body of a hard gelatine capsule. The capsule can be coated with multiple layers of polymers. The capsule can be coated first with an acid soluble polymer, such as EUDRAGIT® E, then with a hydrophilic polymer, such as hydroxypropyl methylcellulose, and finally with an enteric coating, such as EUDRAGIT® L.

An additional example of a suitable time-controlled delivery system is the CHRONOTROPIC® system, or a variant thereof, which comprises a drug core that is coated with hydroxypropyl methylcellulose and an outer enteric film.

An additional example of a suitable time-controlled delivery system is the CODES™ system, or a variant thereof. The time-controlled delivery system can comprise a capsule body, which can house, for example, a drug-containing tablet, an erodible tablet, a swelling expulsion excipient, or any combination thereof. The capsule can comprise an ethyl cellulose coat. The time-controlled delivery system can comprise two different sized capsules, one inside the other. The space between the capsules can comprise a hydrophilic polymer. The drug-containing core canay be housed within the inner capsule. The drug delivery system can comprise an impermeable shell, a drug-containing core, and erodible outer layers at each open end. When the outer layers erode, the drug is released.

Examples of suitable multiparticulate drug delivery systems include DIFFUCAPS®, DIFFUTAB®, ORBEXA®, EURAND MINITABS®, MICROCAPS®, and variants thereof. The drug delivery system can comprise multiparticulate beads, which are comprised of multiple layers of the drug compound, excipients, and release-controlling polymers. The multiparticulate beads can comprise an organic acid or alkaline buffer. The multiparticulate beads can comprise a solid solution of the drug compound and crystallization inhibitor. The drug delivery system can comprise a matrix tablet containing water-soluble particles and the drug compound. The matrix tablet can further comprise hydrophilic and hydrophobic polymers. In some multiparticulate delivery systems, particles in the micron size range are used. In some multiparticulate delivery systems, nanoparticle colloidal carriers composed of natural or synthetic polymers are used.

In some embodiments, a controlled release formulation is a delayed release form. A delayed release form can be formulated to delay a compound's action for an extended period of time. A delayed release form can be formulated to delay the release of an effective dose of one or more compounds, for example, for about 4, about 5, about 6, about 7, about 8, about 9, about 10, about 11, about 12, about 13, about 14, about 15, about 16, about 17, about 18, about 19, about 20, about 21, about 22, about 23, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 5, about 6, about 7, about 8, about 9, about 10, about 11, about 12, about 13, about 14, about 15, about 16, about 17, about 18, about 19, about 20, about 21, about 22, about 23, or about 24 hours.

A tablet providing a sustained or controlled release can comprise a first layer containing one or two of the compounds described herein, and a tablet core containing one or two other compounds. The core can have a delayed or sustained dissolution rate. Other exemplary embodiments can include a barrier between the first layer and core, to limit drug release from the surface of the core. Barriers can prevent dissolution of the core when the pharmaceutical formulation is first exposed to gastric fluid. For example, a barrier can comprise a disintegrant, a dissolution-retarding coating (e.g., a polymeric material, for example, an enteric polymer such as a Eudragit polymer), or a hydrophobic coating or film, and can be selectively soluble in either the stomach or intestinal fluids. Such barriers permit the compounds to leach out slowly. The barriers can cover substantially the whole surface of the core.

In some embodiments, excipients are selected from the comprising granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, plant cellulosic material and spheronization agents, and any combination thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 2000 mg; from about 5 mg to about 1000 mg, from about 10 mg to about 25 mg to 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, CW137 is present in a composition in an amount ranging from about 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

In some embodiments, CW231 is present in a composition in an amount ranging from about 1 mg/kg to about 300 mg/kg, about 2 mg/kg to about 200 mg/kg, about 3 mg/kg to about 100 mg/kg, about 5 mg/kg to about 75 mg/kg, about 10 mg/kg to about 50 mg/kg or about 20 mg/kg to about 40 mg/kg

In some embodiments, a composition comprises from about 100 mg/day to about 800 mg/day of CW137, from about 5 mg/day to about 80 mg/day of CW231.

In some embodiments, a dose can be expressed in terms of an amount of the drug divided by the mass of the subject, for example, milligrams of drug per kilograms of subject body mass.

In some embodiments, CW147 is present in a composition in an amount ranging from about 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg. In some embodiments, CW231 is present in a composition in an amount ranging from about 1 mg/kg to about 300 mg/kg, about 2 mg/kg to about 200 mg/kg, about 3 mg/kg to about 100 mg/kg, about 5 mg/kg to about 75 mg/kg, about 10 mg/kg to about 50 mg/kg or about 20 mg/kg to about 40 mg/kg

In some embodiments, a composition comprises from about 100 mg/day to about 800 mg/day of CW147, from about 5 mg/day to about 80 mg/day of CW231.

In some embodiments, a compound described herein is present in a composition in an amount that is a fraction or percentage of the maximum tolerated amount. The maximum tolerated amount can be as determined in a subject, such as a mouse or human. The fraction can be expressed as a ratio of the amount present in the composition divided by the maximum tolerated dose. The ratio can be from about 1/20 to about 1/1. The ratio can be about 1/20, about 1/19, about 1/18, about 1/17, about 1/16, about 1/15, about 1/14, about 1/13, about 1/12, about 1/11, about 1/10, about 1/9, about 1/8, about 1/7, about 1/6, about 1/5, about 1/4, about 1/3, about 1/2, or about 1/1. The ratio can be 1/20, 1/19, 1/18, 1/17, 1/16, 1/15, 1/14, 1/13, 1/12, 1/11, 1/10, 1/9, 1/8, 1/7, 1/6, 1/5, 1/4, 1/3, 1/2, or 1/1. The ratio can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The ratio can be in a range from about 5% to about 100%, from about 10% to about 100%, from about 5% to about 80%, from about 10% to about 80%, from about 5% to about 60%, from about 10% to about 60%, from about 5% to about 50%, from about 10% to about 50%, from about 5% to about 40%, from about 10% to about 40%, from about 5% to about 20%, or from about 10% to about 20%.

### Pharmacokinetic and Pharmacodynamic Measurements.

Pharmacokinetic and pharmacodynamic data can be obtained by various experimental techniques. Appropriate pharmacokinetic and pharmacodynamic profile components describing a particular composition can vary due to variations in drug metabolism in human subjects. Pharmacokinetic and pharmacodynamic profiles can be based on the determination of the mean parameters of a group of subjects. The group of subjects includes any reasonable number of subjects suitable for determining a representative mean, for example, 5 subjects, 10 subjects, 15 subjects, 20 subjects, 25 subjects, 30 subjects, 35 subjects, or more. The mean is determined by calculating the average of all subject's measurements for each parameter measured. A dose can be modulated to achieve a desired pharmacokinetic or pharmacodynamics profile, such as a desired or effective blood profile, as described herein.

The pharmacodynamic parameters can be any parameters suitable for describing compositions of the invention. For example, the pharmacodynamic profile can be obtained at a time after dosing of, for example, about zero minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about zero hours, about 0.5 hours, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, or about 24 hours.

The pharmacokinetic parameters can be any parameters suitable for describing a compound. The Cₘₐₓ can be, for example, not less than about 1 ng/mL; not less than about 5 ng/mL; not less than about 10 ng/mL; not less than about 15 ng/mL; not less than about 20 ng/mL; not less than about 25 ng/mL; not less than about 50 ng/mL; not less than about 75 ng/mL; not less than about 100 ng/mL; not less than about 200 ng/mL; not less than about 300 ng/mL; not less than about 400 ng/mL; not less than about 500 ng/mL; not less than about 600 ng/mL; not less than about 700 ng/mL; not less than about 800 ng/mL; not less than about 900 ng/mL; not less than about 1000 ng/mL; not less than about 1250 ng/mL; not less than about 1500 ng/mL; not less than about 1750 ng/mL; not less than about 2000 ng/mL; or any other Cₘₐₓ appropriate for describing a pharmacokinetic profile of a compound described herein. The Cₘₐₓ can be, for example, about 1 ng/mL to about 5,000 ng/mL; about 1 ng/mL to about 4,500 ng/mL; about 1 ng/mL to about 4,000 ng/mL; about 1 ng/mL to about 3,500 ng/mL; about 1 ng/mL to about 3,000 ng/mL; about 1 ng/mL to about 2,500 ng/mL; about 1 ng/mL to about 2,000 ng/mL; about 1 ng/mL to about 1,500 ng/mL; about 1 ng/mL to about 1,000 ng/mL; about 1 ng/mL to about 900 ng/mL; about 1 ng/mL to about 800 ng/mL; about 1 ng/mL to about 700 ng/mL; about 1 ng/mL to about 600 ng/mL; about 1 ng/mL to about 500 ng/mL; about 1 ng/mL to about 450 ng/mL; about 1 ng/mL to about 400 ng/mL; about 1 ng/mL to about 350 ng/mL; about 1 ng/mL to about 300 ng/mL; about 1 ng/mL to about 250 ng/mL; about 1 ng/mL to about 200 ng/mL; about 1 ng/mL to about 150 ng/mL; about 1 ng/mL to about 125 ng/mL; about 1 ng/mL to about 100 ng/mL; about 1 ng/mL to about 90 ng/mL; about 1 ng/mL to about 80 ng/mL; about 1 ng/mL to about 70 ng/mL; about 1 ng/mL to about 60 ng/mL; about 1 ng/mL to about 50 ng/mL; about 1 ng/mL to about 40 ng/mL; about 1 ng/mL to about 30 ng/mL; about 1 ng/mL to about 20 ng/mL; about 1 ng/mL to about 10 ng/mL; about 1 ng/mL to about 5 ng/mL; about 10 ng/mL to about 4,000 ng/mL; about 10 ng/mL to about 3,000 ng/mL; about 10 ng/mL to about 2,000 ng/mL; about 10 ng/mL to about 1,500 ng/mL; about 10 ng/mL to about 1,000 ng/mL; about 10 ng/mL to about 900 ng/mL; about 10 ng/mL to about 800 ng/mL; about 10 ng/mL to about 700 ng/mL; about 10 ng/mL to about 600 ng/mL; about 10 ng/mL to about 500 ng/mL; about 10 ng/mL to about 400 ng/mL; about 10 ng/mL to about 300 ng/mL; about 10 ng/mL to about 200 ng/mL; about 10 ng/mL to about 100 ng/mL; about 10 ng/mL to about 50 ng/mL; about 25 ng/mL to about 500 ng/mL; about 25 ng/mL to about 100 ng/mL; about 50 ng/mL to about 500 ng/mL; about 50 ng/mL to about 100 ng/mL; about 100 ng/mL to about 500 ng/mL; about 100 ng/mL to about 400 ng/mL; about 100 ng/mL to about 300 ng/mL; or about 100 ng/mL to about 200 ng/mL.

The Tₘₐₓ of a compound described herein can be, for example, not greater than about 0.5 hours, not greater than about 1 hours, not greater than about 1.5 hours, not greater than about 2 hours, not greater than about 2.5 hours, not greater than about 3 hours, not greater than about 3.5 hours, not greater than about 4 hours, not greater than about 4.5 hours, not greater than about 5 hours, or any other Tₘₐₓ appropriate for describing a pharmacokinetic profile of a compound described herein. The Tₘₐₓ can be, for example, about 0.1 hours to about 24 hours; about 0.1 hours to about 0.5 hours; about 0.5 hours to about 1 hour; about 1 hour to about 1.5 hours; about 1.5 hours to about 2 hour; about 2 hours to about 2.5 hours; about 2.5 hours to about 3 hours; about 3 hours to about 3.5 hours; about 3.5 hours to about 4 hours; about 4 hours to about 4.5 hours; about 4.5 hours to about 5 hours; about 5 hours to about 5.5 hours; about 5.5 hours to about 6 hours; about 6 hours to about 6.5 hours; about 6.5 hours to about 7 hours; about 7 hours to about 7.5 hours; about 7.5 hours to about 8 hours; about 8 hours to about 8.5 hours; about 8.5 hours to about 9 hours; about 9 hours to about 9.5 hours; about 9.5 hours to about 10 hours; about 10 hours to about 10.5 hours; about 10.5 hours to about 11 hours; about 11 hours to about 11.5 hours; about 11.5 hours to about 12 hours; about 12 hours to about 12.5 hours; about 12.5 hours to about 13 hours; about 13 hours to about 13.5 hours; about 13.5 hours to about 14 hours; about 14 hours to about 14.5 hours; about 14.5 hours to about 15 hours; about 15 hours to about 15.5 hours; about 15.5 hours to about 16 hours; about 16 hours to about 16.5 hours; about 16.5 hours to about 17 hours; about 17 hours to about 17.5 hours; about 17.5 hours to about 18 hours; about 18 hours to about 18.5 hours; about 18.5 hours to about 19 hours; about 19 hours to about 19.5 hours; about 19.5 hours to about 20 hours; about 20 hours to about 20.5 hours; about 20.5 hours to about 21 hours; about 21 hours to about 21.5 hours; about 21.5 hours to about 22 hours; about 22 hours to about 22.5 hours; about 22.5 hours to about 23 hours; about 23 hours to about 23.5 hours; or about 23.5 hours to about 24 hours.

The AUC_{(0-inf)} of a compound described herein can be, for example, not less than about 1 ng·hr/mL, not less than about 5 ng·hr/mL, not less than about 10 ng·hr/mL, not less than about 20 ng·hr/mL, not less than about 30 ng·hr/mL, not less than about 40 ng·hr/mL, not less than about 50 ng·hr/mL, not less than about 100 ng·hr/mL, not less than about 150 ng·hr/mL, not less than about 200 ng·hr/mL, not less than about 250 ng·hr/mL, not less than about 300 ng·hr/mL, not less than about 350 ng·hr/mL, not less than about 400 ng·hr/mL, not less than about 450 ng·hr/mL, not less than about 500 ng·hr/mL, not less than about 600 ng·hr/mL, not less than about 700 ng·hr/mL, not less than about 800 ng·hr/mL, not less than about 900 ng·hr/mL, not less than about 1000 ng·hr/mL, not less than about 1250 ng·hr/mL, not less than about 1500 ng·hr/mL, not less than about 1750 ng·hr/mL, not less than about 2000 ng·hr/mL, not less than about 2500 ng·hr/mL, not less than about 3000 ng·hr/mL, not less than about 3500 ng·hr/mL, not less than about 4000 ng·hr/mL, not less than about 5000 ng·hr/mL, not less than about 6000 ng·hr/mL, not less than about 7000 ng·hr/mL, not less than about 8000 ng·hr/mL, not less than about 9000 ng·hr/mL, not less than about 10,000 ng·hr/mL, or any other AUC_{(0-inf)} appropriate for describing a pharmacokinetic profile of a compound described herein. The AUC_{(0-inf)} of a compound can be, for example, about 1 ng·hr/mL to about 10,000 ng·hr/mL; about 1 ng·hr/mL to about 10 ng·hr/mL; about 10 ng·hr/mL to about 25 ng·hr/mL; about 25 ng·hr/mL to about 50 ng·hr/mL; about 50 ng·hr/mL to about 100 ng·hr/mL; about 100 ng·hr/mL to about 200 ng·hr/mL; about 200 ng·hr/mL to about 300 ng·hr/mL; about 300 ng·hr/mL to about 400 ng·hr/mL; about 400 ng·hr/mL to about 500 ng·hr/mL; about 500 ng·hr/mL to about 600 ng·hr/mL; about 600 ng·hr/mL to about 700 ng·hr/mL; about 700 ng·hr/mL to about 800 ng·hr/mL; about 800 ng·hr/mL to about 900 ng·hr/mL; about 900 ng·hr/mL to about 1,000 ng·hr/mL; about 1,000 ng·hr/mL to about 1,250 ng·hr/mL; about 1,250 ng·hr/mL to about 1,500 ng·hr/mL; about 1,500 ng·hr/mL to about 1,750 ng·hr/mL; about 1,750 ng·hr/mL to about 2,000 ng·hr/mL; about 2,000 ng·hr/mL to about 2,500 ng·hr/mL; about 2,500 ng·hr/mL to about 3,000 ng·hr/mL; about 3,000 ng·hr/mL to about 3,500 ng·hr/mL; about 3,500 ng·hr/mL to about 4,000 ng·hr/mL; about 4,000 ng·hr/mL to about 4,500 ng·hr/mL; about 4,500 ng·hr/mL to about 5,000 ng·hr/mL; about 5,000 ng·hr/mL to about 5,500 ng·hr/mL; about 5,500 ng·hr/mL to about 6,000 ng·hr/mL; about 6,000 ng·hr/mL to about 6,500 ng·hr/mL; about 6,500 ng·hr/mL to about 7,000 ng·hr/mL; about 7,000 ng·hr/mL to about 7,500 ng·hr/mL; about 7,500 ng·hr/mL to about 8,000 ng·hr/mL; about 8,000 ng·hr/mL to about 8,500 ng·hr/mL; about 8,500 ng·hr/mL to about 9,000 ng·hr/mL; about 9,000 ng·hr/mL to about 9,500 ng·hr/mL; or about 9,500 ng·hr/mL to about 10,000 ng·hr/mL.

The plasma concentration of a compound described herein can be, for example, not less than about 1 ng/mL, not less than about 5 ng/mL, not less than about 10 ng/mL, not less than about 15 ng/mL, not less than about 20 ng/mL, not less than about 25 ng/mL, not less than about 50 ng/mL, not less than about 75 ng/mL, not less than about 100 ng/mL, not less than about 150 ng/mL, not less than about 200 ng/mL, not less than about 300 ng/mL, not less than about 400 ng/mL, not less than about 500 ng/mL, not less than about 600 ng/mL, not less than about 700 ng/mL, not less than about 800 ng/mL, not less than about 900 ng/mL, not less than about 1000 ng/mL, not less than about 1200 ng/mL, or any other plasma concentration of a compound described herein. The plasma concentration can be, for example, about 1 ng/mL to about 2,000 ng/mL; about 1 ng/mL to about 5 ng/mL; about 5 ng/mL to about 10 ng/mL; about 10 ng/mL to about 25 ng/mL; about 25 ng/mL to about 50 ng/mL; about 50 ng/mL to about 75 ng/mL; about 75 ng/mL to about 100 ng/mL; about 100 ng/mL to about 150 ng/mL; about 150 ng/mL to about 200 ng/mL; about 200 ng/mL to about 250 ng/mL; about 250 ng/mL to about 300 ng/mL; about 300 ng/mL to about 350 ng/mL; about 350 ng/mL to about 400 ng/mL; about 400 ng/mL to about 450 ng/mL; about 450 ng/mL to about 500 ng/mL; about 500 ng/mL to about 600 ng/mL; about 600 ng/mL to about 700 ng/mL; about 700 ng/mL to about 800 ng/mL; about 800 ng/mL to about 900 ng/mL; about 900 ng/mL to about 1,000 ng/mL; about 1,000 ng/mL to about 1,100 ng/mL; about 1,100 ng/mL to about 1,200 ng/mL; about 1,200 ng/mL to about 1,300 ng/mL; about 1,300 ng/mL to about 1,400 ng/mL; about 1,400 ng/mL to about 1,500 ng/mL; about 1,500 ng/mL to about 1,600 ng/mL; about 1,600 ng/mL to about 1,700 ng/mL; about 1,700 ng/mL to about 1,800 ng/mL; about 1,800 ng/mL to about 1,900 ng/mL; or about 1,900 ng/mL to about 2,000 ng/mL.

The present disclosure further relates to a method for treating a subject either suspected of having or having condition or mutation or a combination thereof, wherein said condition is selected from solid tumor such as Pancreatic Cancer, Colorectal Cancer, Lung Cancer, Brain Cancer, Head and Neck Cancer, Breast cancer and Liquid tumor such as multiple myeloma, acute non lymphocytic leukemia and myelodysplasia, or any combination of conditions thereof, or wherein said mutation is in the RAS genes alone or in combination with mutation in other genes that can promote cancer, such as, BRAF, EGFR, β-catenin, CDKN2A, P13KCA, TP53, APC, MYC, BCL2, SOCS1, SMAD4 etc or any combination of mutations thereof, the method comprising administering to the subject: a composition of CW137 and one of the compound selected from CW231, or any combination thereof, optionally along with pharmaceutically acceptable excipient(s); or a compositions of CW147 and CW231, optionally along with pharmaceutically acceptable excipient(s).

In some embodiments, the compounds are administered as a composition of CW137 and CW231 or a composition of CW147 and CW231 at time intervals ranging from about 1 second to about 64800 seconds. Each compound can be administered at a dosage of about an IC₁₀ to about an IC₈₀ dose.

The oncology disease system used for virtual experiments herein is a comprehensive representation of the bio-molecular activity involved in solid and liquid tumors. The system includes various pathways and bio-molecular interactions in the key phenotypes of cancer, such as viability, apoptosis, proliferation, angiogenesis, and metastasis.

The system provides a dynamic and kinetic representation of the signaling pathways underlying tumor physiology at the bio-molecular level with coverage of various tumor phenotypes, including proliferation, viability, angiogenesis, metastasis, apoptosis, tumor metabolism, and the tumor microenvironment related to associated inflammation. Also included are protein players and associated gene and mRNA species associated with tumor related signaling. Signaling pathways comprising growth factors such as EGFR, PDGFRA, FGFR, c-MET, VEGFR and IGF-1R, cell cycle regulators, mTOR signaling, p53 signaling, cytokine pathways IL1, IL4, IL6, IL12, IL15 TNF; TGF-β, hypoxia-mediated regulation, angiogenic promoters, lipid mediators and tumor metabolism are represented. Kinases and transcription factors associated with the tumor physiology network are represented. The modeling of the time-dependent changes in the fluxes of the constituent pathways is done utilizing modified ordinary differential equations (ODE) and mass action kinetics. The current version of the technology includes over 5000 biological species with over thirty thousand cross-talk interactions. The platform is prospectively and retrospectively validated against an extensive set of pre-defined in vitro and in vivo studies.

The starting control state of the system can be based on normal epithelial cell physiology that is non-tumorigenic. The user can control the transition of the normal system to a neoplastic disease state aligning with specific tumor mutation profiles. This control is accomplished, for example, through over-expression of the tumorigenic genes EGFR, IGF-1R; knock-downs of the tumor-suppressors p53, PTEN; and increased states of hypoxia and oxidative stress. Knockdowns or over-expressions of biological species can be done at the expression or activity levels.

Drugs can be represented in this technology through explicit mechanism of action specification. The drug concentration in the virtual experiments can be explicitly assumed to be post ADME (Absorption, Distribution, Metabolism, and Excretion). Multiple virtual patient profiles can be generated by overlaying the functional impact of mutations. Therapeutic combinations can be tested against this panel of virtual patients to understand the differential sensitivity of the therapy to the multiple patient profiles.

In some embodiments, the composition and the process of preparing the composition for the purpose of improving one or more undesirable symptoms associated with the disease states or for slowing the progression (worsening) of one or more symptoms associated with the disease is provided.

Therapeutic combinations can comprise at least one member of a first group and at least one member of a second group wherein members of the first group are selected from a Raf inhibitor and c-MET inhibitor; and members of the second group are selected from a HMG CoA reductase inhibitor or ATP Citrate Lyase inhibitor or Farnesyltransferase inhibitor or Geranylgeranyltransferase inhibitor or Farnesyl diphosphate synthatase inhibitors. The therapeutic combination can comprise: at least one Raf inhibitor (CW137) and at least one HMG CoA reductase inhibitor or ATP Citrate Lyase inhibitor or Farnesyltransferase inhibitor or Geranylgeranyltransferase inhibitor or Farnesyl diphosphate synthatase inhibitors (CW231); or at least one c-MET inhibitor (CW147) and at least one HMG CoA reductase inhibitor or ATP Citrate Lyase inhibitor or Farnesyltransferase inhibitor or Geranylgeranyltransferase inhibitor or Farnesyl diphosphate synthatase inhibitors (CW231).

### Physicochemical Properties and Mechanism of Action.

CW137

Name: Sorafenib

Drug Bank Accession No: DB00398 (APRD01304, DB07438)

Type: Small Molecule inhibitor

Groups: Approved

Description: Sorafenib (rINN), marketed as Nexavar™, is a drug approved for the treatment of advanced renal cell carcinoma (primary kidney cancer); has also received “Fast Track” designation by the FDA for the treatment of advanced hepatocellular carcinoma (primary liver cancer), and has since performed well in Phase III trials. Sorafenib is a small molecular inhibitor of RAF kinase, PDGF (platelet-derived growth factor), VEGF receptor 2 & 3 kinases, and c-Kit. Sorafenib can simultaneously target the RAF/Mek/Erk pathway.

Brand names: Nexavar™

Chemical Formula: C₂₁H₁₆ClF₃N₄O₃

IUPAC Name: 4-[4-[[4-chloro-3-(trifluoromethyl)phenyl]carbamoylamino]phenoxy]-N-methylpyridine-2-carboxamide.

Indication: For the treatment of patients with advanced renal cell carcinoma.

Pharmacodynamics: Sorafenib is a multikinase inhibitor targeting several serine/threonine and receptor tyrosine kinases, commonly available as a tosylate salt. Sorafenib is a multikinase inhibitor that can decrease tumor cell proliferation in vitro. Sorafenib can inhibit tumor growth of murine renal cell carcinoma (RENCA) and several other human tumor xenografts in athymic mice. A reduction in tumor angiogenesis can occur in some tumor xenograft models.

Mechanism of action: Sorafenib interacts with multiple intracellular (CRAF, BRAF and mutant BRAF) and cell surface kinases (KIT, FLT-3, VEGFR-2, VEGFR-3, and PDGFR-β). Several of these kinases can be involved in angiogenesis; thus, sorafenib can reduce blood flow to the tumor. Sorafenib can target the RAF/Mek/Erk pathway. By inhibiting these kinases, genetic transcription of proteins involved in cell proliferation and angiogenesis can be inhibited.

Absorption: The mean relative bioavailability is about 38-49% for the tablet form, when compared to an oral solution. With a high-fat meal, bioavailability is reduced by about 29% compared to administration in the fasted state.

Metabolism: Sorafenib is metabolized primarily in the liver by CYP3A4-mediated oxidative metabolism, as well as glucuronidation mediated by UGT1A9. Sorafenib accounts for approximately 70-85% of the circulating analytes in plasma at steady-state. Eight metabolites of sorafenib have been identified, of which five have been detected in the plasma. The main circulating metabolite of sorafenib in plasma, the pyridine N-oxide, shows in vitro potency similar to that of sorafenib. This metabolite comprises approximately 9-16% of circulating analytes at steady-state.

Half-life: 25-48 hours.

Route of elimination: Following oral administration of a 100 mg dose of a solution formulation of sorafenib, 96% of the dose can be recovered within 14 days, with 77% of the dose excreted in feces, and 19% of the dose excreted in urine as glucuronidated metabolites.

Class of Drugs involved in this category: Vemurafenib, GDC-0879, PLX4720, Regorafenib, RAF265, Raf265, NVP-BHG712, SB590885, ZM 336372, AZ628.

CW147

Name: ARQ-197 (TIVANTINIB)

Type: Small Molecule Inhibitor

Group: Phase 3

Description: An oral small molecule inhibitor, which can inhibit c-Met selectively in a non-competitive manner. c-Met is a receptor tyrosine kinase (RTK), which is activated by HGF. c-Met plays an important role in cancer by activating different oncogenic pathways including RAS, PI3K, and STAT3. c-Met activation can lead to the invasive growth of tumor cells.

Pharmacodynamics: ARQ-197 can cause a decrease in phosphorylated c-Met, total c-Met, and phosphorylated FAK. ARQ-197 can also cause a significant increase in TUNEL staining.

MOA: ARQ-197 inhibits c-Met in a non-ATP-competitive manner.

Absorption: CaCo-2 flux data suggests good permeability, and pharmacokinetic studies in multiple species show an oral bioavailability greater than about 20%. These favorable ADME (absorption, distribution, metabolism, elimination) and preclinical data predict good oral bioavailability in patients, which is confirmed by the clinical pharmacokinetics data of ARQ-197 in cancer patients. The parent compound, tivantinib, is not detected in urine and only trace amounts are detected in feces. The fact that the urinary and fecal metabolites are oxidative and subsequently conjugated molecules suggests near complete absorption of tivantinib when administered under fed conditions.

Metabolism: ARQ 197 is metabolized by the cytochrome P450 CYP2C19 enzyme, which is subject to genetic polymorphisms.

Half-Life: Median time to maximal concentration (tₘₐₓ) for tivantinib in plasma and total radioactivity in plasma and whole blood are 4, 6, and 6 hours, respectively. Mean terminal half-life (t_{1/2}) for tivantinib in plasma and total radioactivity in plasma and whole blood were 11.7, 20.7, and 13.6 hours, respectively.

Route of Elimination: Mean±SD recovery of radioactivity in feces and urine is 87.2% (feces=68.2%±3.80%; urine=19.0%±2.42%). Parent tivantinib is not detected in urine and trace amounts were detected in feces. Urinary and fecal metabolites are oxidative and subsequently conjugated molecules.

Class of Drugs involved in this category: PF-04217903, JNJ-38877605, PHA-665752, SU11274, INCB28060, AMG-208, AMG-337, NVP-BVU972, BMS-777607, SGX-523.

CW147

Name: CRIZOTINIB

Type: Small Molecule Inhibitor

Group: Approved

Description: Crizotinib is a multikinase inhibitor which can preferentially inhibit c-MET and ALK.

Brand Name: Xalkori™

Indication: Crizotinib is a kinase inhibitor indicated for the treatment of patients with metastatic non-small cell lung cancer (NSCLC) whose tumors are anaplastic lymphoma kinase (ALK)-positive as detected by an FDA-approved test.

Pharmacodynamics: The QT interval prolongation potential of crizotinib was assessed in all patients who received XALKORI™ 250 mg twice daily. Serial ECGs in triplicate were collected following a single dose and at steady state to evaluate the effect of crizotinib on QT intervals. Sixteen of 1167 patients (1.4%) were found to have QTcF (corrected QT by the Fridericia method) greater than or equal to 500 msec and 51 of 1136 patients (4.4%) had an increase from baseline QTcF greater than or equal to 60 msec by automated machine-read evaluation of ECG. A pharmacokinetic/pharmacodynamic analysis suggested a concentration-dependent increase in QTcF.

MOA: Crizotinib is an inhibitor of receptor tyrosine kinases including, c-MET, ALK, ROS1 (c-ros), and Recepteur d'Origine Nantais (RON). Translocations can affect the ALK gene resulting in the expression of oncogenic fusion proteins. The formation of ALK fusion proteins can result in activation and dysregulation of gene expression and signaling, and can contribute to increased cell proliferation and survival in tumors expressing these proteins. Crizotinib can show concentration-dependent inhibition of ALK, ROS1, and c-Met phosphorylation in cell-based assays using tumor cell lines, and can also show antitumor activity in mice bearing tumor xenografts that express EML4- or NPM-ALK fusion proteins or c-Met.

Absorption: Following a single oral dose, crizotinib was absorbed with median time to achieve peak concentration of 4 to 6 hours. Following crizotinib 250 mg twice daily, a steady state was reached within 15 days and remained stable, with a median accumulation ratio of 4.8. Steady-state systemic exposure (Cₘᵢₙ and AUC) appeared to increase in a greater than dose proportional manner over the dose range of 200-300 mg twice daily. The mean absolute bioavailability of crizotinib was 43% (range: 32% to 66%) following a single 250 mg oral dose. A high-fat meal reduced crizotinib AUC_{inf} and Cₘₐₓ by approximately 14%. XALKORI™ can be administered with or without food.

Metabolism: Crizotinib is predominantly metabolized by CYP3A4/5. The primary metabolic pathways in humans are oxidation of the piperidine ring to crizotinib lactam and O-dealkylation, with subsequent Phase 2 conjugation of O-dealkylated metabolites.

Half Life: Following single doses of crizotinib, the mean apparent plasma terminal half-life of crizotinib is 42 hours in patients.

Route of Elimination: Following the administration of a single 250 mg radiolabeled crizotinib dose to healthy subjects, 63% and 22% of the administered dose was recovered in feces and urine, respectively. Unchanged crizotinib represented approximately 53% and 2.3% of the administered dose in feces and urine, respectively. The mean apparent clearance (CL/F) of crizotinib was lower at steady state (60 L/h) after 250 mg twice daily than that after a single 250 mg oral dose (100 L/h), which was likely due to autoinhibition of CYP3A by crizotinib after multiple dosing.

Class of Drugs involved in this category: ARQ-197, PF-04217903, JNJ-38877605, PHA-665752, SU11274, INCB28060, AMG-208, AMG-337, NVP-BVU972, BMS-777607, SGX-523

CW231a

Name: SIMVASTATIN

Drug Bank ID: DB00641 (APRD00104)

Type: small molecule

Group: Approved

Description: A derivative of lovastatin and a potent competitive inhibitor of 3-hydroxy-3-methylglutaryl coenzyme A reductase (hydroxymethylglutaryl COA reductases), which is the rate-limiting enzyme in cholesterol biosynthesis. Simvastatin can also interfere with steroid hormone production and due to the induction of hepatic LDL receptors, and can increase the breakdown of LDL cholesterol.

Brand Names: Cholestat™, Coledis, Colemin, Corolin, Denan™, Labistatin, Lipex™, Lodales, Medipo, Nivelipol™, Pantok™, Rendapid™, Simovil™, Sinvacor™, Sivastin™, Synvinolin, Vasotenal, Zocor™, Zocord

Indication: For the treatment of hypercholesterolemia.

Pharmacodynamics: Simvastatin, the methylated form of lovastatin, is an oral antilipemic agent that can inhibit HMG-CoA reductase. Simvastatin is used in the treatment of primary hypercholesterolemia and is effective in reducing total and LDL-cholesterol as well as plasma triglycerides and apolipoprotein B.

Mechanism of Action: The 6-membered lactone ring of simvastatin is hydrolyzed in vivo to generate the beta, delta-dihydroxy acid, an active metabolite structurally similar to HMG-CoA (hydroxymethylglutaryl CoA). Once hydrolyzed, simvastatin competes with HMG-CoA for HMG-CoA reductase, a hepatic microsomal enzyme. Interference with the activity of this enzyme reduces the quantity of mevalonic acid, a precursor of cholesterol.

Absorption: Absorption of simvastatin, estimated relative to an intravenous reference dose, in each of two animal species tested, averaged about 85% of an oral dose. In animal studies, after oral dosing, simvastatin achieved substantially higher concentrations in the liver than in non-target tissues.

Metabolism: Hepatic, simvastatin is a substrate for CYP3A4.

Half-life: 3 hours.

Route of Elimination: Following an oral dose of ¹⁴C-labeled simvastatin in humans, 13% of the dose was excreted in urine and 60% in feces.

Class of Drugs involved in this category: Atorvastatin, Cerivastatin, Fluvastatin, Lovastatin, Mevastatin, Pitavastatin, Pravastatin, Rosuvastatin.

CW231a

Name: Atorvastatin

Drug Bank Accession No: DB01076 (APRD00055)

Type: Small Molecule inhibitor

Groups: Approved

Description: Atorvastatin (Lipitor™) is a member of the statin drug class and can be used for lowering cholesterol. Atorvastatin is a competitive inhibitor of hydroxymethylglutaryl-coenzyme A (HMG-CoA) reductase, the rate-determining enzyme in cholesterol biosynthesis via the mevalonate pathway. HMG-CoA reductase catalyzes the conversion of HMG-CoA to mevalonate. Atorvastatin acts primarily in the liver. Decreased hepatic cholesterol levels increase hepatic uptake of cholesterol and reduce plasma cholesterol levels.

Brand names: Lipitor™

Chemical Formula: C₃₃H₃₅FN₂O

IUPAC Name: (3R,5R)-7-[2-(4-fluorophenyl)-3-phenyl-4-(phenylcarbamoyl)-5-propan-2-ylpyrrol-1-yl]-3,5-dihydroxyheptanoic acid

Indication: Atorvastatin can be used as primary prevention in individuals with multiple risk factors for coronary heart disease (CHD) and as secondary prevention in individuals with CHD to reduce the risk of myocardial infarction (MI), stroke, angina, and revascularization procedures. Atorvastatin can also be used to reduce the risk of cardiovascular events in patients with acute coronary syndrome (ACS). Atorvastatin can also be used in the treatment of primary hypercholesterolemia and mixed dyslipidemia, homozygous familial hypercholesterolemia, primary dysbetalipoproteinemia, and/or hypertriglyeridemia as an adjunct to dietary therapy to decrease serum total and low-density lipoprotein cholesterol (LDL-C), apolipoprotein B (apoB), and triglyceride concentrations, while increasing high-density lipoprotein cholesterol (HDL-C) levels.

Pharmacodynamics: Atorvastatin, a selective, competitive HMG-CoA reductase inhibitor, can be used to lower serum total and LDL cholesterol, apoB, and triglyceride levels while increasing HDL cholesterol. High LDL-C, low HDL-C and high TG concentrations in the plasma are associated with an increased risk of atherosclerosis and cardiovascular disease. The total cholesterol to HDL-C ratio is a strong predictor of coronary artery disease and high ratios are associated with a higher risk of disease. Increased levels of HDL-C are associated with lower cardiovascular risk. By decreasing LDL-C and TG and increasing HDL-C, atorvastatin can reduce the risk of cardiovascular morbidity and mortality. Atorvastatin has a unique structure, long half-life, and hepatic selectivity, explaining its greater LDL-lowering potency compared to other HMG-CoA reductase inhibitors.

Mechanism of action: Atorvastatin can selectively and competitively inhibit the hepatic enzyme HMG-CoA reductase. As HMG-CoA reductase is responsible for converting HMG-CoA to mevalonate in the cholesterol biosynthesis pathway, the action of atorvastatin can result in a subsequent decrease in hepatic cholesterol levels; decreased hepatic cholesterol levels stimulate upregulation of hepatic LDL-C receptors, which can increase hepatic uptake of LDL-C and reduce serum LDL-C concentrations.

Absorption: Atorvastatin is rapidly absorbed after oral administration with maximum plasma concentrations achieved in about 1 to 2 hours. The absolute bioavailability of atorvastatin (parent drug) is approximately 14% and the systemic availability of the HMG-CoA reductase inhibitory activity is approximately 30%. The low systemic bioavailability is due to presystemic clearance by gastrointestinal mucosa and first-pass metabolism in the liver.

Metabolism: Atorvastatin is extensively metabolized to ortho- and parahydroxylated derivatives and various beta-oxidation products. In vitro inhibition of HMG-CoA reductase by ortho- and parahydroxylated metabolites is equivalent to that of atorvastatin. Approximately 70% of circulating inhibitory activity for HMG-CoA reductase is attributed to active metabolites. CYP3A4 is also involved in the metabolism of atorvastatin.

Half-life: 14 hours, but the half-life of HMG-CoA inhibitor activity is about 20-30 hours due to longer-lived active metabolites

Route of elimination: Eliminated primarily in the bile after hepatic and/or extrahepatic metabolism. Does not appear to undergo significant enterohepatic recirculation. Less than 2% of the orally administered dose is recovered in urine.

Class of Drugs involved in this category: Simvastatin, Cerivastatin, Fluvastatin, Lovastatin, Mevastatin, Pitavastatin, Pravastatin, Rosuvastatin.

CW231b

Name: SB-204990

Type: Small Molecule Inhibitor

Group: Preclinical.

Description: SB-204990 is a lactone prodrug of SB-201076, which is a potent inhibitor of ATP citrate lyase (ACLY). SB-204990 inhibited cholesterol synthesis and fatty acid synthesis in Hep G2 cells (dose-related inhibition of up to 91% and 82% respectively) and rats (76% and 39% respectively).

Pharmacodynamics: SB-204990 does not inhibit ATP citrate-lyase directly, but is absorbed by the hepatocytes and hydrolysed to the potent ATP citrate-lyase inhibitor, SB-201076, within these cells.

Mechanism of Action: SB-204990 acts as a prodrug for SB-201076. SB-201076 is a competitive inhibitor of ATP citrate lyase with a K_{I} of 1 μM in humans.

Absorption: When administered orally, SB-204990 is absorbed into the systemic circulation and concentrated into the liver, where substantial amounts of both SB-204990 and SB-201076 can be detected. Sufficient concentrations of SB-201076 can be achieved in the liver, relative to the K_{I} for ATP citrate-lyase, to inhibit ATP citrate lyase enzyme activity effectively.

CW231c

Name: Tipifamib

Drug Bank Accession No: DB04960

Type: Small Molecule inhibitor

Groups: Investigational

Description: Tipifamib is being studied in the treatment of acute myeloid leukemia (AML) and other types of cancer, is a farnesyltransferase inhibitor, and is also called R115777 and Zarnestra™.

Brand names: Zarnestra™

Chemical Formula: C₂₇H₂₂Cl₂N₄O

IUPAC Name: 6-[(R)-amino-(4-chlorophenyl)-(3-methylimidazol-4-yl)methyl]-4-(3-chlorophenyl)-1-methylquinolin-2-one

Indication: Investigated for use/treatment in colorectal cancer, leukemia (myeloid), pancreatic cancer, and solid tumors.

Pharmacodynamics: Tipifamib is a potent, selective, and competitive inhibitor of the enzyme farnesyltransferase (FTase). This enzyme is important in the processing and activation of signalling molecules linked to cell proliferation and malignant transformation, such as RAS, Rho-B, Rac, and lamin proteins. Inhibition of FTase by tipifarnib can induce antileukaemic and antitumoral activity, which has been demonstrated in both in vitro and in vivo animal models. The nature of the cellular and tumor tissue responses elicited by tipifarnib treatment in vivo is consistent with the hypothesis that the antitumor effects are being derived from disruption of multiple effectors downstream of FTase inhibition.

Mechanism of action: The farnesyltransferase inhibitors (FTIs) are a class of experimental cancer drugs that target protein farnesyltransferase with the downstream effect of preventing the proper functioning of the Ras protein, which is commonly abnormally active in cancer. After translation, RAS goes through four steps of modification: isoprenylation, proteolysis, methylation and palmitoylation. Isoprenylation involves the enzyme farnesyltransferase (FTase) transferring a farnesyl group from farnesyl pyrophosphate (FPP) to the pre-RAS protein. Also, a related enzyme geranylgeranyltransferase I (GGTase I) has the ability to transfer a geranylgeranyl group to K and N-RAS. Farnesyl is necessary to attach RAS to the cell membrane. Without attachment to the cell membrane, RAS is not able to transfer signals from membrane receptors.

Absorption: Tipifamib is absorbed rapidly, and maximum plasma concentrations are reached within two to three hours following oral administration. The absolute oral bioavailability of tipifarnib, administered to healthy volunteers under fed conditions, can be 34%.

Metabolism: In vitro experiments indicate that cytochrome P450 enzymes (CYP) 3A4, 2C19, 2A6, 2D6, and 2C8/9/10 play a role in the oxidative biotransformation of tipifarnib. In addition, tipifarnib undergoes direct glucuronidation. The concentrations of metabolites identified in plasma are markedly lower compared to those of tipifarnib. One exception is the glucuronide metabolite of tipifarnib, which exhibits plasma concentrations several-fold higher relative to the parent compound. Several metabolites identified thus far, including the tipifarnib-glucuronide, are either inactive as inhibitors of farnesyltransferase or have much lower activity relative to the parent compound. Thus, the antiproliferative effects of tipifarnib are believed to be directly related to the parent compound. The population analyses suggest that genetic polymorphisms of the CYP enzymes have no clinically relevant impact on the pharmacokinetics of tipifarnib. Consistent with the hepatic metabolism of tipifarnib, the population pharmacokinetic analysis indicated a weak but statistically significant correlation between total serum bilirubin concentration and tipifarnib clearance. For illustrative purposes, a two-fold increase in bilirubin concentration is associated with a 6.9% decrease in tipifarnib clearance. The small magnitude of this effect suggests that adjustment of the tipifarnib dose on the basis of serum bilirubin concentrations is not normally necessary.

Half-life: 2-5 hours.

Route of elimination: Tipifamib's pharmacokinetic profile is consistent with a three-compartment disposition model. Urinary excretion of unchanged tipifarnib or its metabolites accounts for 13.7% of the total dose administered. Thus, impaired renal function is expected to have no clinically relevant impact on the pharmacokinetics of tipifarnib.

Class of Drugs involved in this category: Lonafarnib, BMS-214662, L778123, L-731734, B1086, L-744832, BIM-46228, FTI-276, RPR-130401, FTI-2148, FTI-2628, FTI-277, FTase Inhibitor I, FTase Inhibitor II, FTase Inhibitor III, L-745631, L-739749

### Cancer and Associated Conditions and Methods of Treatment.

In some embodiments, the invention provides a use of compounds in preparing a medicament for the treatment of a condition, the compounds comprising: a kinsase inhibitor or a pharmaceutically-acceptable salt thereof; and an inhibitor of an enzyme that is involved in lipid metabolism or a pharmaceutically-acceptable salt thereof.

In some embodiments, the invention provides a use of compounds in treatment of a condition, the compounds comprising: a kinsase inhibitor or a pharmaceutically-acceptable salt thereof; and an inhibitor of an enzyme that is involved in lipid metabolism or a pharmaceutically-acceptable salt thereof.

In some embodiments, the disclosure provides a process for preparing a composition, the composition comprising one or more compounds, wherein the compounds are CW137, CW147, are chosen from CW231, wherein the composition optionally further comprises a pharmaceutically-acceptable excipient, wherein the process comprises the step of combining the compounds and the optional excipient in any order thereof.

The disclosure provides therapeutic methods for the treatment of cancer and associated conditions, or combinations thereof.

In some embodiments, the disclosure provides a method for treating a subject either suspected of having or having a condition or mutation or a combination thereof, wherein said condition is selected from cancers with RAS mutations including lung cancer, colorectal cancer, pancreatic cancer, glioblastoma, multiple myeloma, or any combination thereof, or wherein said mutation co-occurs with other cancer-promoting mutations, such as those in genes such as BRAF, EGFR, β-catenin, CDKN2A, P13KCA, TP53, APC, MYC, BCL2, SOCS1, SMAD4, or any combination thereof, the method comprising administering to the subject a combination of a) a therapeutically-effective amount of a RAF inhibitor or a c-MET inhibitor; and b) a therapeutically-effective amount of a HMG-CoA reductase inhibitor or ATP citrate lyase inhibitor or Farnesyl diphosphate synthase inhibitor or Farnesyltransferase inhibitor or Geranylgeranyl transferase inhibitor; and wherein the administration uses one or a plurality of dosage forms, each dosage form comprising one or more inhibitors, and wherein each dosage form optionally further comprises a pharmaceutically-acceptable excipient.

In some embodiments the disclosure provides a use of a combination of compounds in the preparation of a medicament for the treatment of cancer and associated conditions, the compounds comprising: a Raf inhibitor or a c-MET inhibitor; and a HMG-CoA reductase inhibitor or ATP citrate lyase inhibitor or Farnesyl diphosphate synthase inhibitor or Farnesyltransferase inhibitor or Geranylgeranyl transferase inhibitor.

In some embodiments the disclosure provides a use of a combination of compounds in the preparation of a kit for the treatment of cancer and associated conditions, the compounds a Raf inhibitor or a c-MET inhibitor; and a HMG-CoA reductase inhibitor or ATP citrate lyase inhibitor or Farnesyl diphosphate synthase inhibitor or Farnesyltransferase inhibitor or Geranylgeranyl transferase inhibitor.

Pharmaceutical compositions containing compounds described herein can be administered for prophylactic and/or therapeutic treatments. In therapeutic applications, the compositions are administered to a subject already suffering from a disease or condition, in an amount sufficient to cure or at least partially arrest the symptoms of the disease or condition, or to cure, heal, improve, or ameliorate the condition itself. Amounts effective for this use can vary based on the severity and course of the disease or condition, previous therapy, the subject's health status, weight, and response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills. The compounds can be packed together or separately, in a single package or in a plurality of packages. One or all of the therapeutic agents can be given in multiple doses. If not simultaneous, the timing between the multiple doses may vary to as much as about a month.

Compounds and compositions of the disclosure can be packaged as a kit. In some embodiments, a kit includes written instructions on the use of the compounds and compositions. The instructions can provide information on the identity of the therapeutic agent(s), modes of administration, or the indications for which the therapeutic agent(s) can be used.

In some embodiments, therapeutics are combined with genetic or genomic testing to determine whether that individual is a carrier of a mutant gene that is known to be correlated with certain diseases or conditions. A personalized medicine approach can be used to provide companion diagnostic tests to discover a subject's predisposition to certain conditions and susceptibility to therapy. For example, a subject who is an anti-EGFR non responder could be identified via companion diagnostics. The companion diagnostic test can be performed on a tumor biopsy. Instructions on the use of a companion diagnostic test can be provided on written material packaged with a compound, composition, or kit of the disclosure. The written material can be, for example, a label. The written material can suggest conditions or genetic features relevant to inflammation or the therapeutic compounds of the disclosure. The instructions provide the subject and the supervising physician with the best guidance for achieving the optimal clinical outcome from the administration of the therapy.

Compounds described herein can be administered before, during, or after the occurrence of a disease or condition, and the timing of administering the composition containing a compound can vary. For example, the compounds can be used as a prophylactic and can be administered continuously to subjects with a propensity to conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A compound can be administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria.

virtual Tumor Based In Vivo Aligned Studies of Therapies of the Disclosure.

The compositions of the present invention were analyzed using a virtual tumor cell system designed to represent the disease state and customized to match the molecular profile of a specified cancer baseline. These experiments were also validated by the examples described later.

The baselines selected for the virtual experiments include:

a) A549 adenocarcinomic human alveolar basal epithelial cells harboring mutations in KRAS;
b) HCT116 human colorectal carcinoma cell line harboring mutations in KRAS and PIK3 CA;
c) H1299 human non small cell lung cancer cell line harboring mutations in NRAS and PTEN;
d) IM9 human multiple myeloma cell line harboring mutations in NRAS;
e) H1650 human alveolar epithelial carcinoma cells harboring mutations in EGFR and PTEN;
f) SW48 human colorectal carcinoma cell line harboring mutations in EGFR;
g) CaCo2 human colorectal cancer cell line harboring mutations in p53 and CTNNB1; and
h) SKMM2 human multiple myeloma cell line harboring mutations in CDKN2C and p53.

In these virtual experiments, the system was first simulated with the oncogenic mutations aligned to the specific profiles of interest and then placed under simulated cultured for a minimum of about 35 hours. The culture time was selected to allow the system to attain a severe oncogenic state through the activation of autocrine and paracrine pathways, presumably affecting all oncogenic mediators including growth factors, kinases and transcription factors. After about 35 hours of simulation, a system customized to the above mentioned tumor profile is created.

The drug compounds CW137, CW147, and CW231 were administered in different combinations to the virtual tumor cell and the cells were placed under simulated cultured for a minimum of about 18-20 hours. The drug administration was performed at multiple dosage ratios across an array of samples for each drug. The effect of the multiple dosage ratios was evaluated after about 18-20 hours of culture by assessing tumor cell survival, apoptosis, and proliferation markers. The major markers assayed include CDK4-CCND1, CDK2-CCNE, CDK2-CCNA and CDK1-CCNB1, which are markers for cell proliferation. Other proteins including BCL2, BIRC5, BIRC2, BAX, CASP3, and cleaved PARP1 were assayed to determine their effect on tumor cell survival and apoptosis. Other vital biomarkers including VEGFA were assayed as a measure of angiogenesis.

Based on the assayed biomarkers, an overall viability score was calculated as a ratio of survival/apoptosis. “Viability” is a scale to measure a change in tumorogenic symptoms. A reduction greater than 30% is considered as being moderately effective and a reduction greater than 50% is considered being an effective therapy.

Viability index is a ratio of a cumulative measure of an average of survival over apoptotic markers and is defined as a ratio of the cumulative survival index over the apoptosis index as shown in TABLE 3. The survival index comprises of AKT, BCL2, BIRC5, BIRC2, MCL1, and XIAP markers. The apoptosis index includes BAX, CASP3, PARP1-cleaved, PMAIP1, CASP8 and BCL2L11.

Proliferation index is a cumulative measure of an average of cell cycle checkpoint complexes including CDK4-CCND1, CDK2-CCNE, CDK2-CCNA, and CDK1-CCNB1 as shown in TABLE 3.

TABLE 3

| Index | Markers | |
|---|---|---|
| Proliferation | CDK4-CCND1, CDK2-CCNE, CDK2-CCNA, | |
| Index | CDC2-CCNB1 | |
| Viability Index = | Survival Markers = | Apoptosis Markers = |
| Survival Index/ | AKT1, BCL2, BIRC5, | BAX, CASP3, |
| Apoptosis Index | BIRC2, MCL1, XIAP | PMAIP1, CASP8, |
| | | BCL2L11 |

The combinations of compounds described herein can provide synergistic and therapeutic benefits at a low dosage.

FIG. 1 illustrates the predicted effect of Sorafenib and Atorvastatin on the proliferation and viability of a non-triggered normal epithelial cell line. The proliferation rate decreased by about 10% while the viability decreased by about 8%, indicating a marginal effect on the proliferation and viability of the normal cells upon administration of the aforementioned compounds.

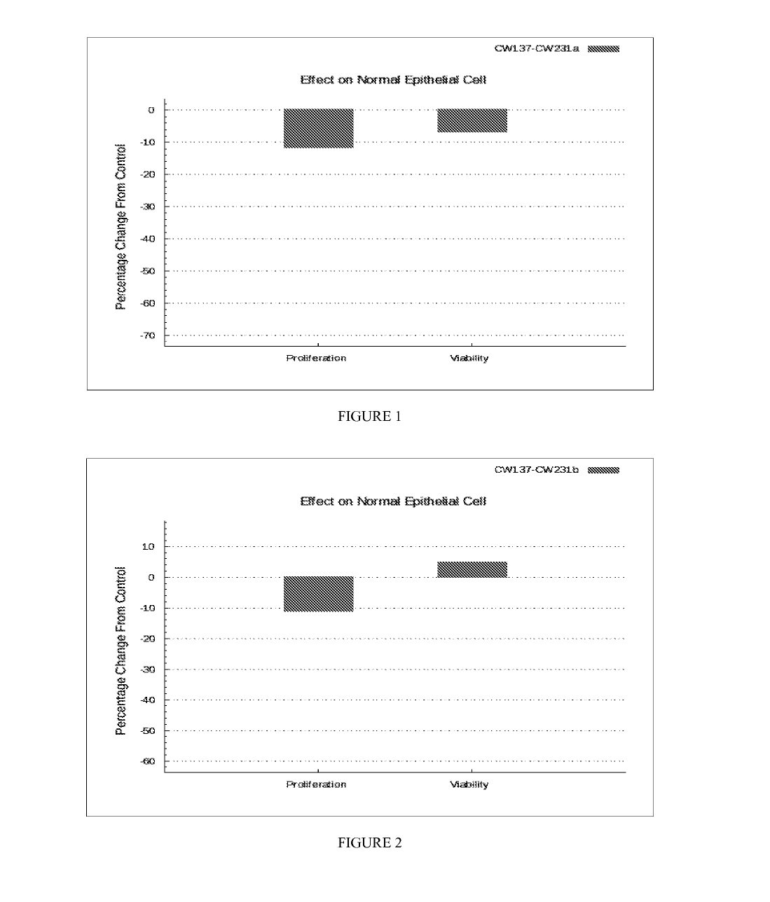

FIG. 2 illustrates the predicted effect of Sorafenib and BMS-303141 on the proliferation and viability of a non-triggered normal epithelial cell line. The proliferation decreased by about 10% while the viability increased by about 4%, indicating a marginal effect on the proliferation and viability of the normal cells upon administration of the aforementioned compounds.

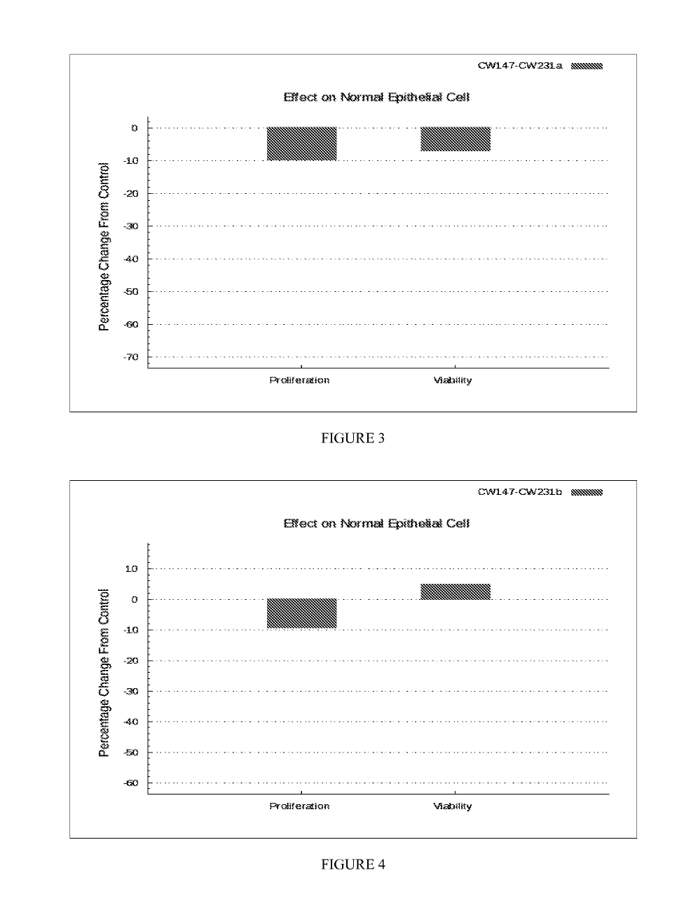

FIG. 3 illustrates the predicted effect of ARQ-197 and Atorvastatin on the proliferation and viability of a non-triggered normal epithelial cell line. The proliferation decreased by about 10% while the viability decreased by about 8%, indicating a marginal effect on the proliferation and viability of the normal cells upon administration of the aforementioned compounds.

FIG. 4 illustrates the predicted effect of ARQ-197 and BMS-303141 on the proliferation and viability of a non-triggered normal epithelial cell line. The proliferation decreased by about 10% while the viability increased by about 5%, indicating a marginal effect on the proliferation and viability of the normal cells upon administration of the aforementioned compounds.

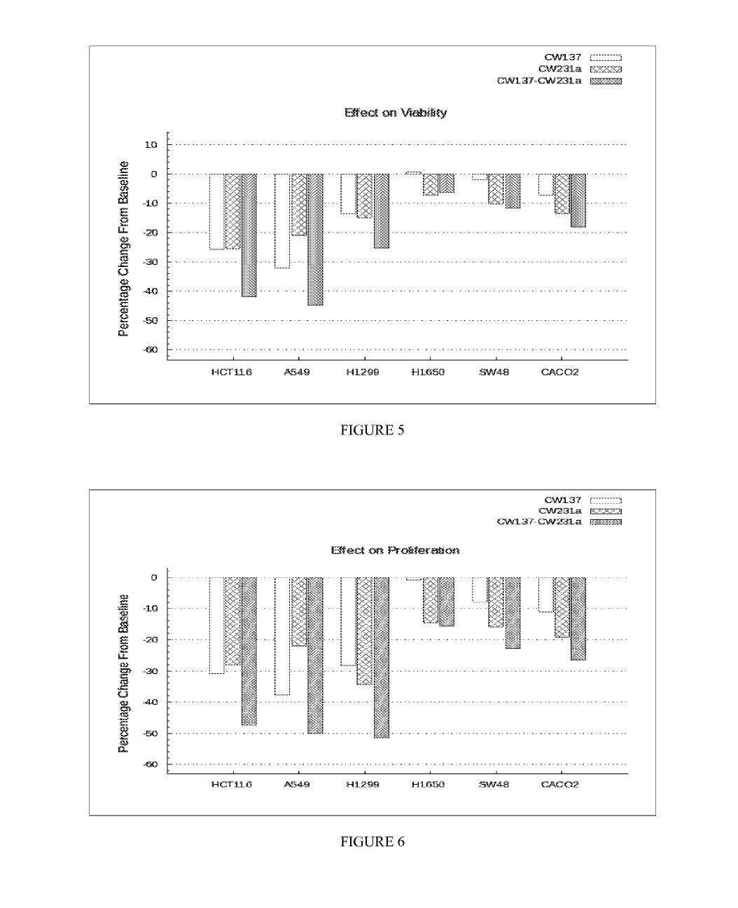

FIG. 5 illustrates the predicted effect of Sorafenib alone, Atorvastatin alone, or Sorafenib and Atorvastatin in combination on the viability of cells with (HCT116, A549, and H1299) or without (H1650, SW48, CaCo2) RAS mutations. When used in the combination, the compounds were used at their IC₂₅ concentrations. When administered alone, the compounds moderately affected the viability of RAS mutant cells, whereas that effect was almost negligible in the cells without RAS mutations. The viability of the RAS mutant cells greatly decreased when the compounds were used in combination, whereas that effect was only marginal in the cells without RAS mutations.

FIG. 6 illustrates the predicted effect of Sorafenib alone, Atorvastatin alone, or Sorafenib and Atorvastatin in combination on the proliferation of cells with (HCT116, A549, and H1299) or without (H1650, SW48, CaCo2) RAS mutations. When used in the combination, the compounds were used at their IC₂₅ concentrations. When administered alone, the compounds moderately affected the proliferation of RAS mutant cells, whereas that effect was almost negligible in the cells without RAS mutations. The proliferation of the RAS mutant cells greatly decreased when the compounds were used in combination, whereas that effect was only marginal in the cells without RAS mutations.

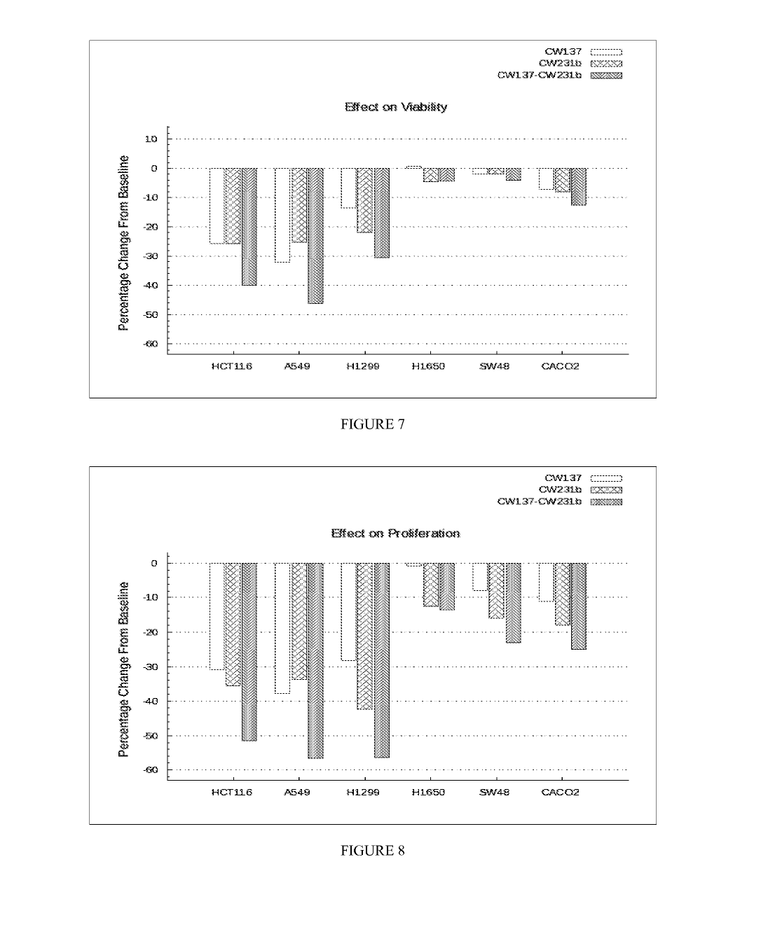

FIG. 7 illustrates the predicted effect of Sorafenib alone, BMS-303141 alone, or Sorafenib and BMS-303141 in combination on the viability of cells with (HCT116, A549, and H1299) or without (H1650, SW48, CaCo2) RAS mutations. When used in the combination, the compounds were used at their IC₂₅ concentrations. When administered alone, the compounds moderately affected the viability of RAS mutant cells, whereas that effect was almost negligible in the cells without RAS mutations. The viability of the RAS mutant cells greatly decreased when the compounds were used in combination, whereas that effect was only marginal in the cells without RAS mutations.

FIG. 8 illustrates the predicted effect of Sorafenib alone, BMS-303141 alone, or Sorafenib and BMS-303141 in combination on the proliferation of cells with (HCT116, A549, and H1299) or without (H1650, SW48, CaCo2) RAS mutations. When used in the combination, the compounds were used at their IC₂₅ concentrations. When administered alone, the compounds moderately affected the proliferation of RAS mutant cells, whereas that effect was almost negligible in the cells without RAS mutations. The proliferation of the RAS mutant cells greatly decreased when the compounds were used in combination, whereas that effect was only marginal in the cells without RAS mutations.

FIG. 9 illustrates the predicted effect of ARQ-197 alone, Atorvastatin alone, or ARQ-197 and Atorvastatin in combination on the viability of cells with (HCT116, A549, and H1299) or without (H1650, SW48, CaCo2) RAS mutations. When used in the combination, the compounds were used at their IC₂₅ concentrations. When administered alone, the compounds moderately affected the viability of RAS mutant cells, whereas that effect was almost negligible in the cells without RAS mutations. The viability of the RAS mutant cells greatly decreased when the compounds were used in combination, whereas that effect was only marginal in the cells without RAS mutations.

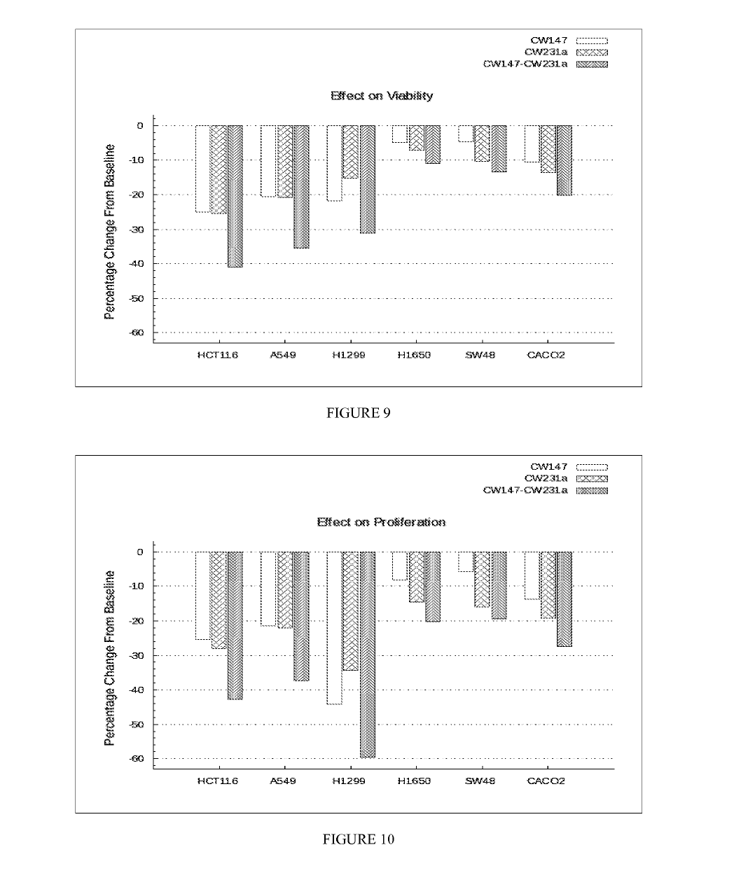

FIG. 10 illustrates the predicted effect of ARQ-197 alone, Atorvastatin alone, or ARQ-197 and Atorvastatin in combination on the proliferation of cells with (HCT116, A549, and H1299) or without (H1650, SW48, CaCo2) RAS mutations. When used in the combination, the compounds were used at their IC₂₅ concentrations. When administered alone, the compounds moderately affected the proliferation of RAS mutant cells, whereas that effect was almost negligible in the cells without RAS mutations. The proliferation of the RAS mutant cells greatly decreased when the compounds were used in combination, whereas that effect was only marginal in the cells without RAS mutations.

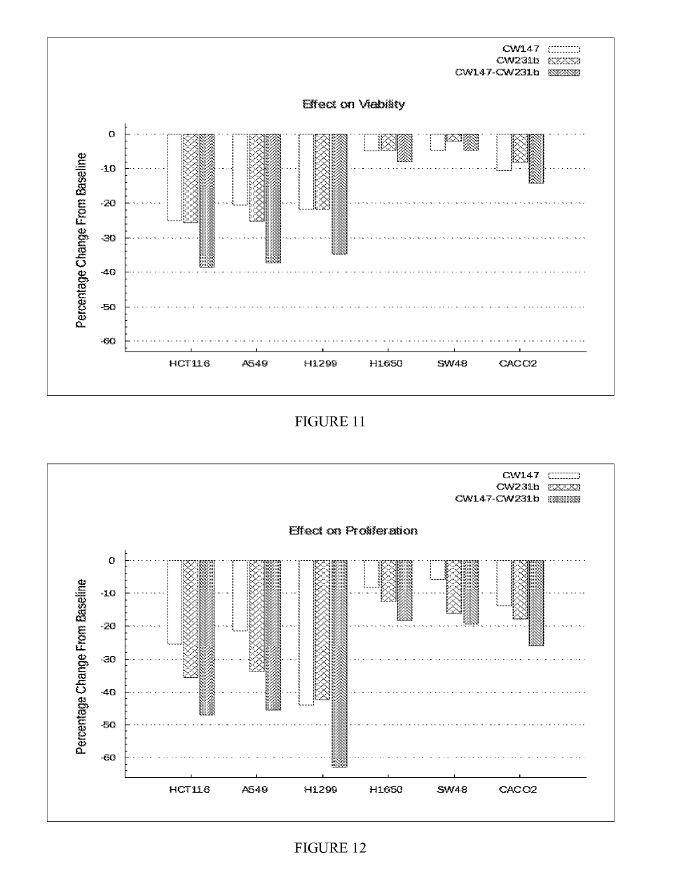

FIG. 11 illustrates the predicted effect of ARQ-197 alone, BMS-303141 alone, or ARQ-197 and BMS-303141 in combination on the viability of cells with (HCT116, A549, and H1299) or without (H1650, SW48, CaCo2) RAS mutations. When used in the combination, the compounds were used at their IC₂₅ concentrations. When administered alone, the compounds moderately affected the viability of RAS mutant cells, whereas that effect was almost negligible in the cells without RAS mutations. The viability of the RAS mutant cells greatly decreased when the compounds were used in combination, whereas that effect was only marginal in the cells without RAS mutations.

FIG. 12 illustrates the predicted effect of ARQ-197 alone, BMS-303141 alone, or ARQ-197 and BMS-303141 in combination on the proliferation of cells with (HCT116, A549, and H1299) or without (H1650, SW48, CaCo2) RAS mutations. When used in the combination, the compounds were used at their IC₂₅ concentrations. When administered alone, the compounds moderately affected the proliferation of RAS mutant cells, whereas that effect was almost negligible in the cells without RAS mutations. The proliferation of the RAS mutant cells greatly decreased when the compounds were used in combination, whereas that effect was only marginal in the cells without RAS mutations.

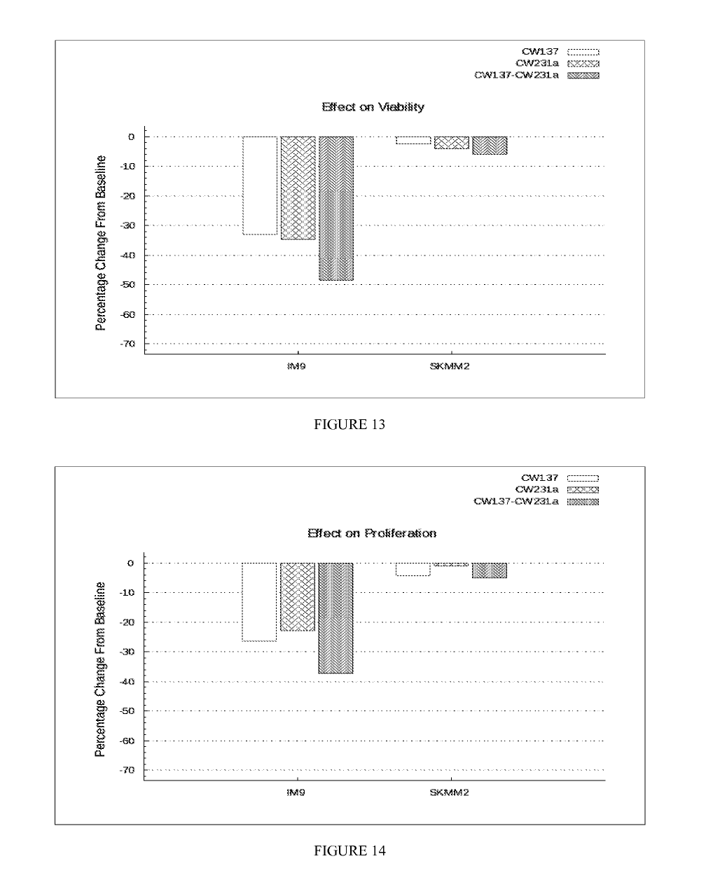

FIG. 13 illustrates the predicted effect of Sorafenib alone, Atorvastatin alone, or Sorafenib and Atorvastatin in combination on the viability of multiple myeloma cell lines with (IM9) or without (SKMM2) RAS mutations. When used in the combination, the compounds were used at their IC₂₅ concentrations. When administered alone, the compounds moderately affected the viability of IM9 cells, whereas that effect was almost negligible in the SKMM2 cells. The viability of IM9 cells greatly decreased when the compounds were used in combination, whereas that effect was only marginal in the SKMM2 cells.

FIG. 14 illustrates the predicted effect of Sorafenib alone, Atorvastatin alone, or Sorafenib and Atorvastatin in combination on the proliferation of multiple myeloma cell lines with (IM9) or without (SKMM2) RAS mutations. When used in the combination, the compounds were used at their IC₂₅ concentrations. When administered alone, the compounds moderately affected the proliferation of IM9 cells, whereas that effect was almost negligible in the SKMM2 cells. The proliferation of IM9 cells was greatly decreased when the compounds were used in combination, whereas that effect was only marginal in the SKMM2 cells.

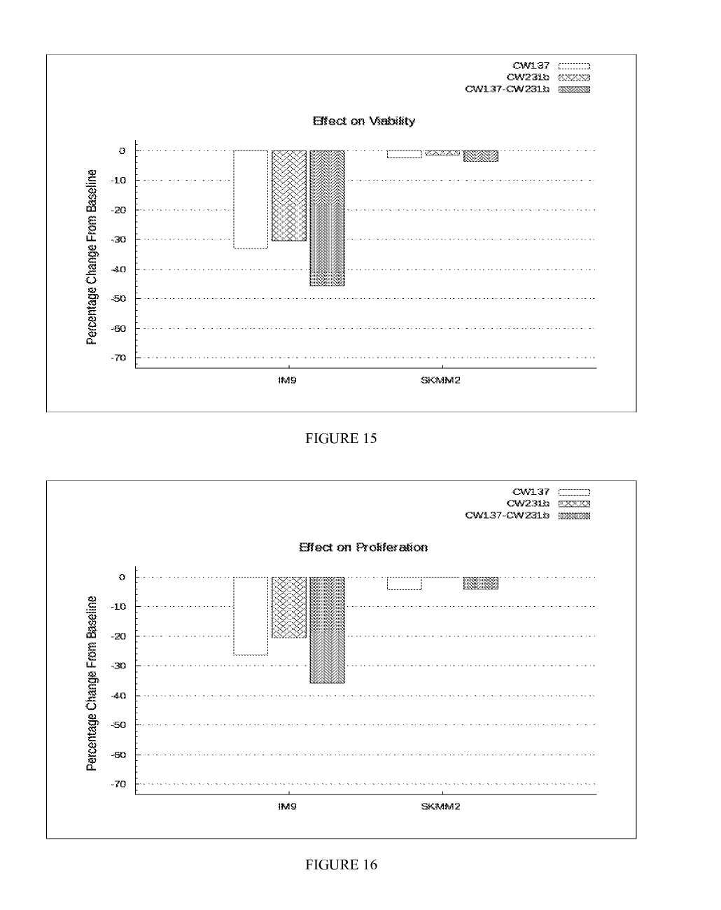

FIG. 15 illustrates the predicted effect of Sorafenib alone, BMS-303141 alone, or Sorafenib and BMS-303141 in combination on the viability of multiple myeloma cell lines with (IM9) or without (SKMM2) RAS mutations. When used in the combination, the compounds were used at their IC₂₅ concentrations. When administered alone, the compounds moderately affected the viability of IM9 cells, whereas that effect was almost negligible in the SKMM2 cells. The viability of IM9 cells was greatly decreased when the compounds were used in combination, whereas that effect was only marginal in the SKMM2 cells.

FIG. 16 illustrates the predicted effect of Sorafenib alone, BMS-303141 alone, or Sorafenib and BMS-303141 in combination on the proliferation of multiple myeloma cell lines with (IM9) or without (SKMM2) RAS mutations. When used in the combination, the compounds were used at their IC₂₅ concentrations. When administered alone, the compounds moderately affected the proliferation of IM9 cells, whereas that effect was almost negligible in the SKMM2 cells. The proliferation of IM9 cells was greatly decreased when the compounds were used in combination, whereas that effect was only marginal in the SKMM2 cells.

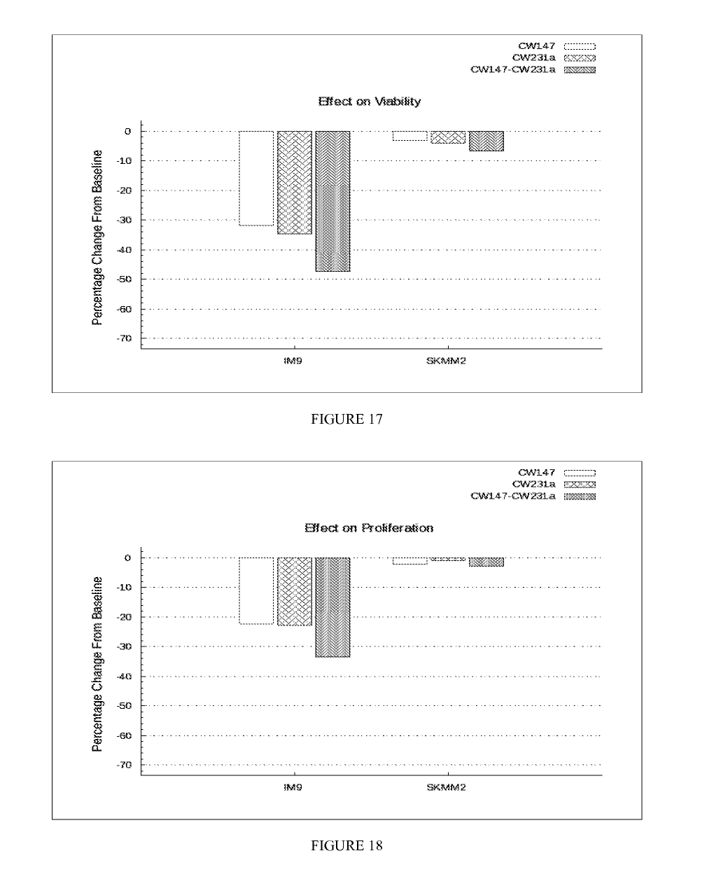

FIG. 17 illustrates the predicted effect of ARQ-197 alone, Atorvastatin alone, or ARQ-197 and Atorvastatin in combination on the viability of multiple myeloma cell lines with (IM9) or without (SKMM2) RAS mutations. When used in the combination, the compounds were used at their IC₂₅ concentrations. When administered alone, the compounds moderately affected the viability of IM9 cells, whereas that effect was almost negligible in the SKMM2 cells. The viability of IM9 cells was greatly decreased when the compounds were used in combination, whereas that effect was only marginal in the SKMM2 cells.

FIG. 18 illustrates the predicted effect of ARQ-197 alone, Atorvastatin alone, or ARQ-197 and Atorvastatin in combination on the proliferation of multiple myeloma cell lines with (IM9) or without (SKMM2) RAS mutations. When used in the combination, the compounds were used at their IC₂₅ concentrations. When administered alone, the compounds moderately affected the proliferation of IM9 cells, whereas that effect was almost negligible in the SKMM2 cells. The proliferation of IM9 cells was greatly decreased when the compounds were used in combination, whereas that effect was only marginal in the SKMM2 cells.

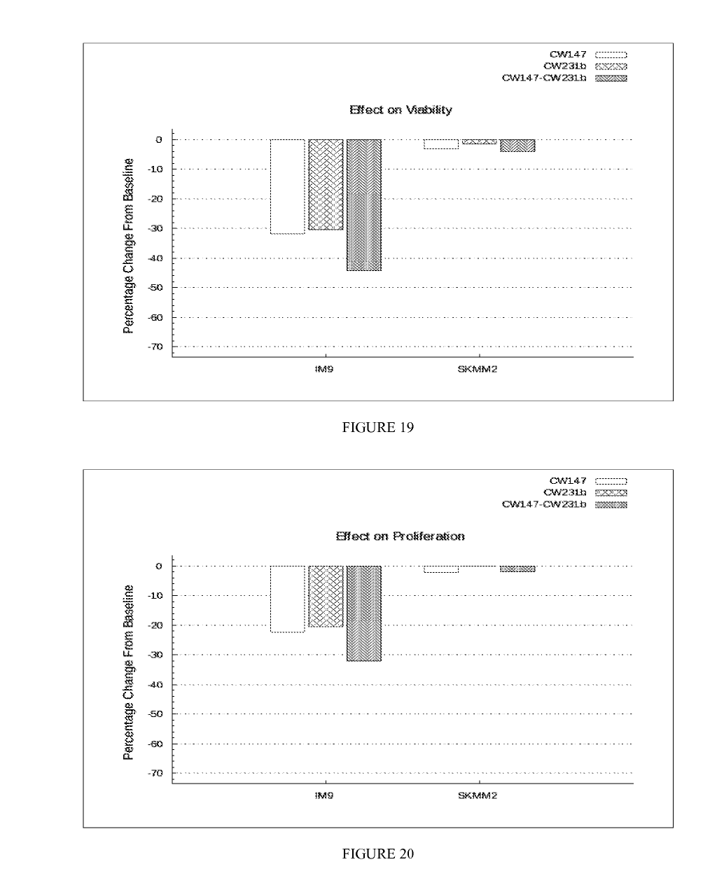

FIG. 19 illustrates the predicted effect of ARQ-197 alone, BMS-303141 alone, or ARQ-197 and BMS-303141 in combination on the viability of multiple myeloma cell lines with (IM9) or without (SKMM2) RAS mutations. When used in the combination, the compounds were used at their IC₂₅ concentrations. When administered alone, the compounds moderately affected the viability of IM9 cells, whereas that effect was almost negligible in the SKMM2 cells. The viability of IM9 cells was greatly decreased when the compounds were used in combination, whereas that effect was only marginal in the SKMM2 cells.

FIG. 20 illustrates the predicted effect of ARQ-197 alone, BMS-303141 alone, or ARQ-197 and BMS-303141 in combination on the proliferation of multiple myeloma cell lines with (IM9) or without (SKMM2) RAS mutations. When used in the combination, the compounds were used at their IC₂₅ concentrations. When administered alone, the compounds moderately affected the proliferation of IM9 cells, whereas that effect was almost negligible in the SKMM2 cells. The proliferation of IM9 cells was greatly decreased when the compounds were used in combination, whereas that effect was only marginal in the SKMM2 cells.

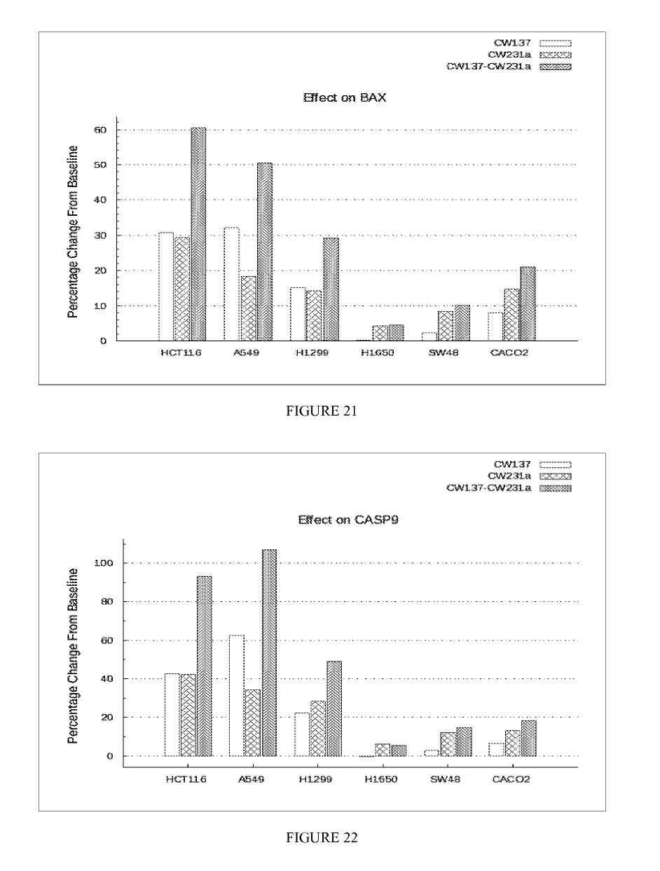

The predicted effect of Sorafenib alone, Atorvastatin alone, or Sorafenib and Atorvastatin in combination on levels of the apoptotic markers BAX (FIG. 21) and CASP9 (FIG. 22) was simulated using cells with (HCT116, A549, and H1299) or without (H1650, SW48, CaCo2) RAS mutations. When used in the combination, the compounds were used at their IC₂₅ concentrations. When administered alone, the compounds moderately affected BAX and CASP9 levels, whereas that effect was almost negligible in the cells without RAS mutations. BAX and CASP9 levels greatly increased when the compounds were used in combination, whereas that effect was only marginal in the cells without RAS mutations.

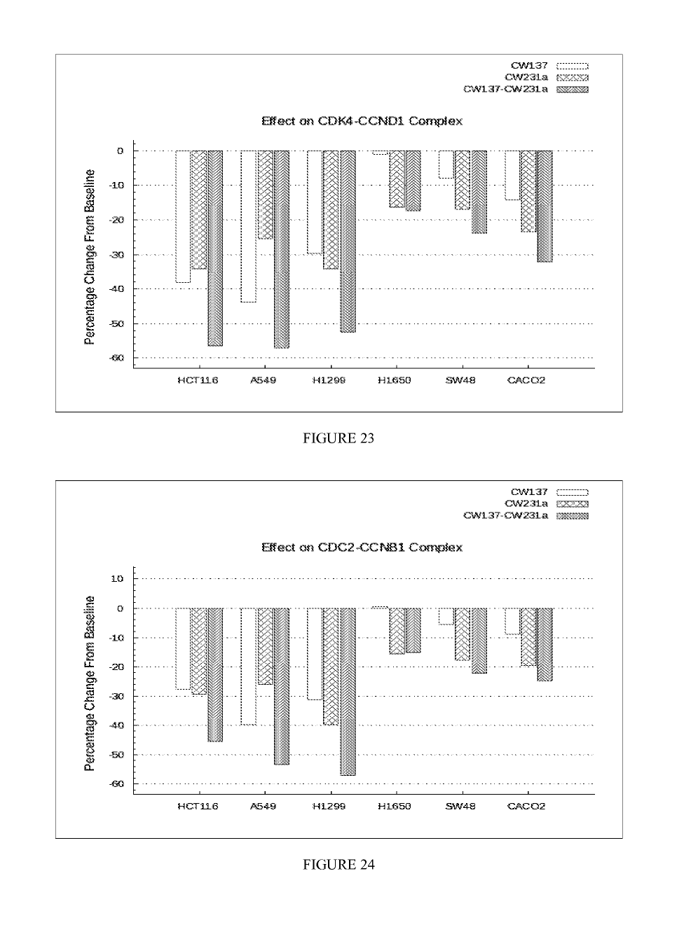

The predicted effect of Sorafenib alone, Atorvastatin alone, or Sorafenib and Atorvastatin in combination on levels of the proliferation markers CDK4-CCND1 (FIG. 23) and CDC2-CCNB1 (FIG. 24) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of the CDK4-CCND1 and CDC2-CCNB1 complexes, whereas that effect was almost negligible in the cells without RAS mutations. CDK4-CCND1 and CDC2-CCNB1 complex levels greatly decreased when the compounds were used in combination, whereas that effect was only marginal in the cells without RAS mutations.

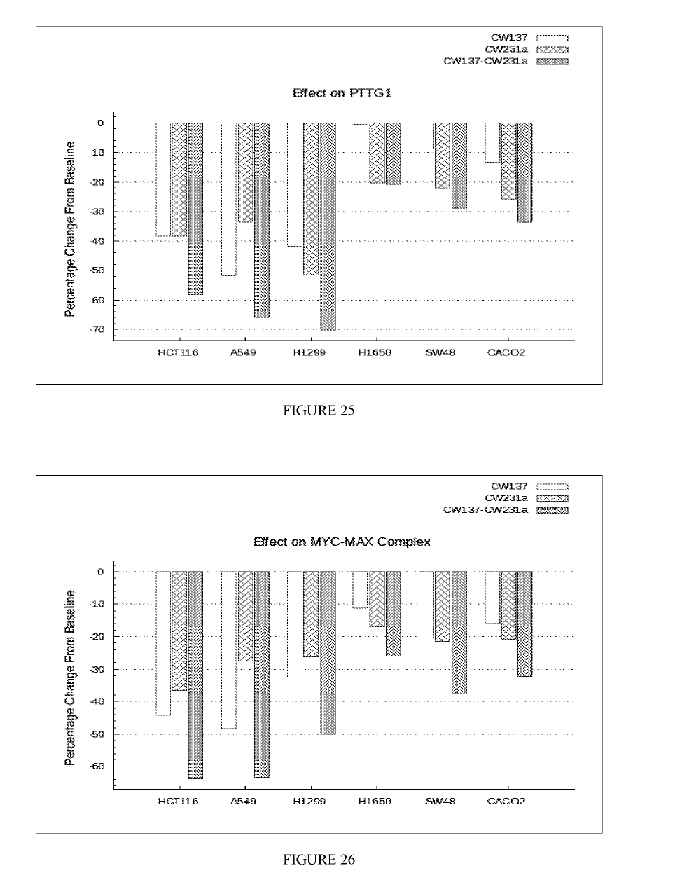

The predicted effect of Sorafenib alone, Atorvastatin alone, or Sorafenib and Atorvastatin in combination on levels of the cell growth markers PTTG1 (FIG. 25) and MYC-MAX (FIG. 26) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of PTTG1 and the MYC-MAX complex, whereas that effect was almost negligible in the cells without RAS mutations. PTTG1 and MYC-MAX levels greatly decreased when the compounds were used in combination, whereas that effect was only marginal in the cells without RAS mutations.

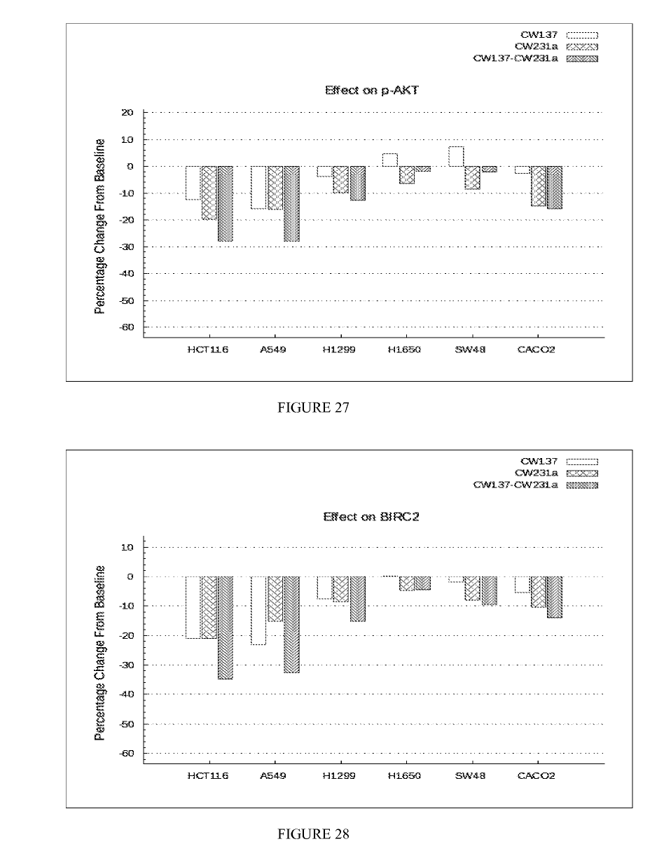

The predicted effect of Sorafenib alone, Atorvastatin alone, or Sorafenib and Atorvastatin in combination on levels of the cell survival markers p-AKT (FIG. 27) and BIRC2 (FIG. 28) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of p-AKT and BIRC2, whereas that effect was almost negligible in the cells without RAS mutations. p-AKT and BIRC2 levels greatly decreased when the compounds were used in combination, whereas that effect was only marginal in the cells without RAS mutations.

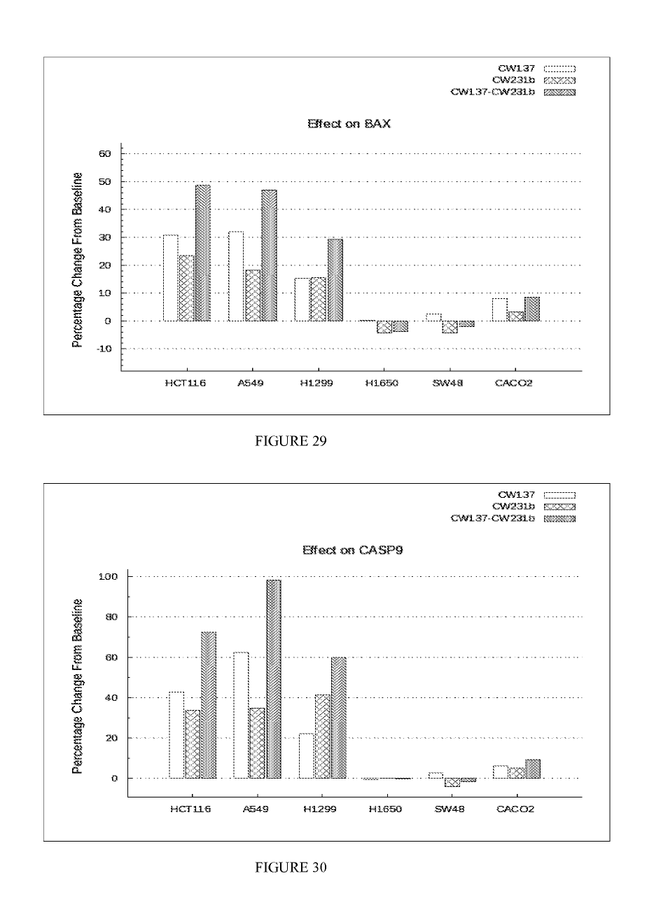

The predicted effect of Sorafenib alone, BMS-303141 alone, or Sorafenib and BMS-303141 in combination on levels of the apoptotic markers BAX (FIG. 29) and CASP9 (FIG. 30) was simulated using cells with (HCT116, A549, and H1299) or without (H1650, SW48, CaCo2) RAS mutations. When used in the combination, the compounds were used at their IC₂₅ concentrations. When administered alone, the compounds moderately affected BAX and CASP9 levels, whereas that effect was almost negligible in the cells without RAS mutations. BAX and CASP9 levels greatly increased when the compounds were used in combination, whereas that effect was only marginal in the cells without RAS mutations.

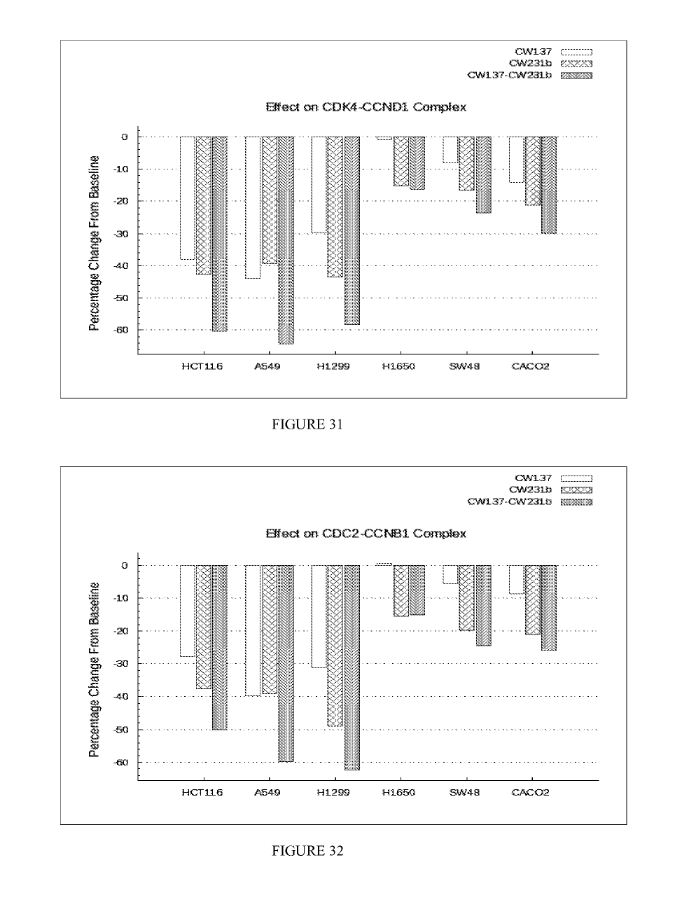

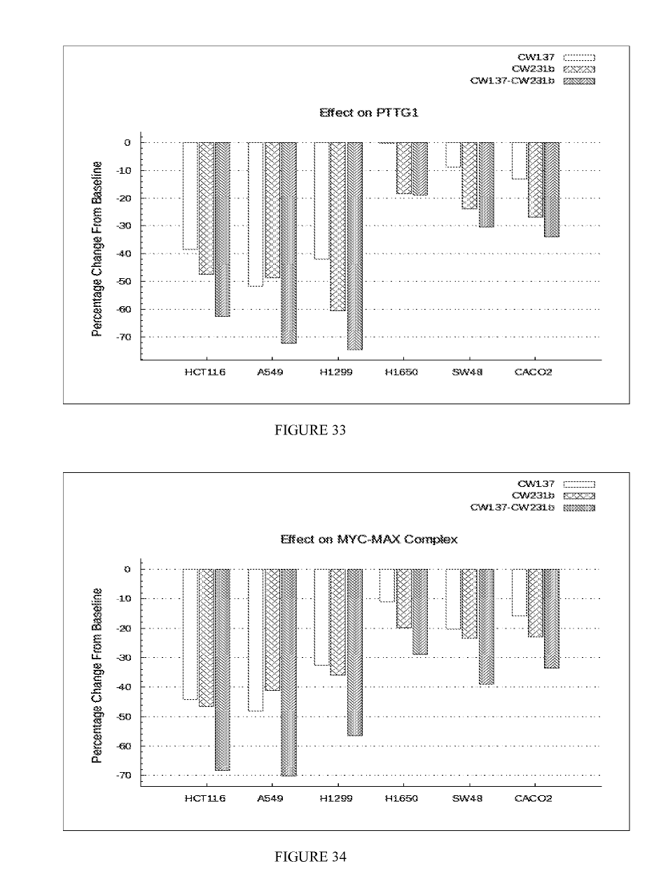

The predicted effect of Sorafenib alone, BMS-303141 alone, or Sorafenib and BMS-303141 in combination on levels of the proliferation markers CDK4-CCND1 (FIG. 31) and CDC2-CCNB1 (FIG. 32) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of the CDK4-CCND1 and CDC2-CCNB1 complexes, whereas that effect was almost negligible in the cells without RAS mutations. CDK4-CCND1 and CDC2-CCNB1 complex levels greatly decreased when the compounds were used in combination, whereas that effect was only marginal in the cells without RAS mutations.

The predicted effect of Sorafenib alone, BMS-303141 alone, or Sorafenib and BMS-303141 in combination on levels of the cell growth markers PTTG1 (FIG. 33) and MYC-MAX (FIG. 34) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of PTTG1 and the MYC-MAX complex, whereas that effect was almost negligible in the cells without RAS mutations. PTTG1 and MYC-MAX levels greatly decreased when the compounds were used in combination, whereas that effect was only marginal in the cells without RAS mutations.

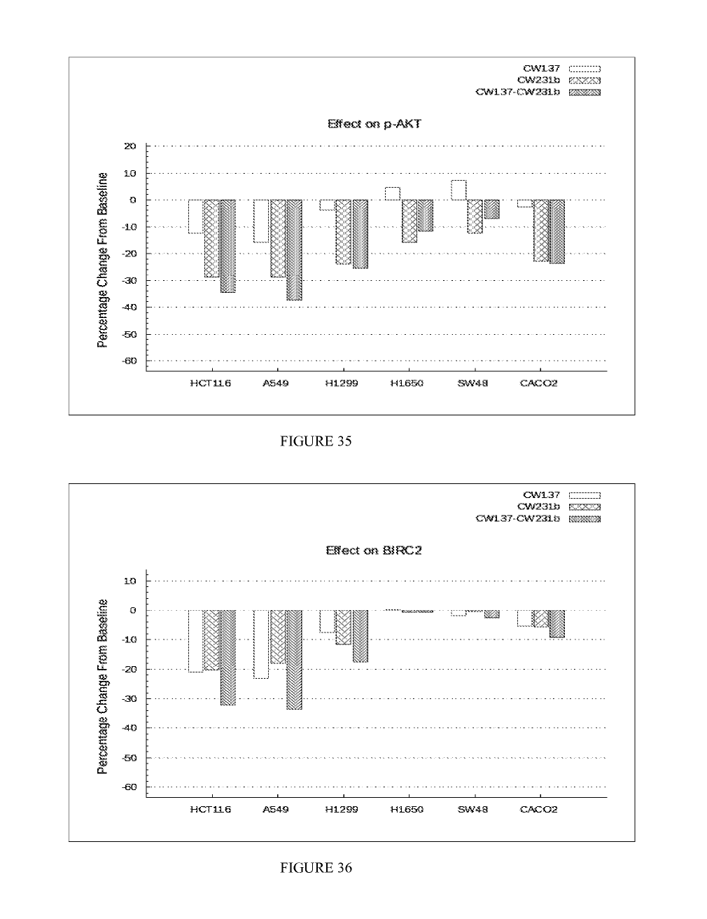

The predicted effect of Sorafenib alone, BMS-303141 alone, or Sorafenib and BMS-303141 in combination on levels of the cell survival markers p-AKT (FIG. 35) and BIRC2 (FIG. 36) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of p-AKT and BIRC2, whereas that effect was almost negligible in the cells without RAS mutations. p-AKT and BIRC2 levels greatly decreased when the compounds were used in combination, whereas that effect was only marginal in the cells without RAS mutations.

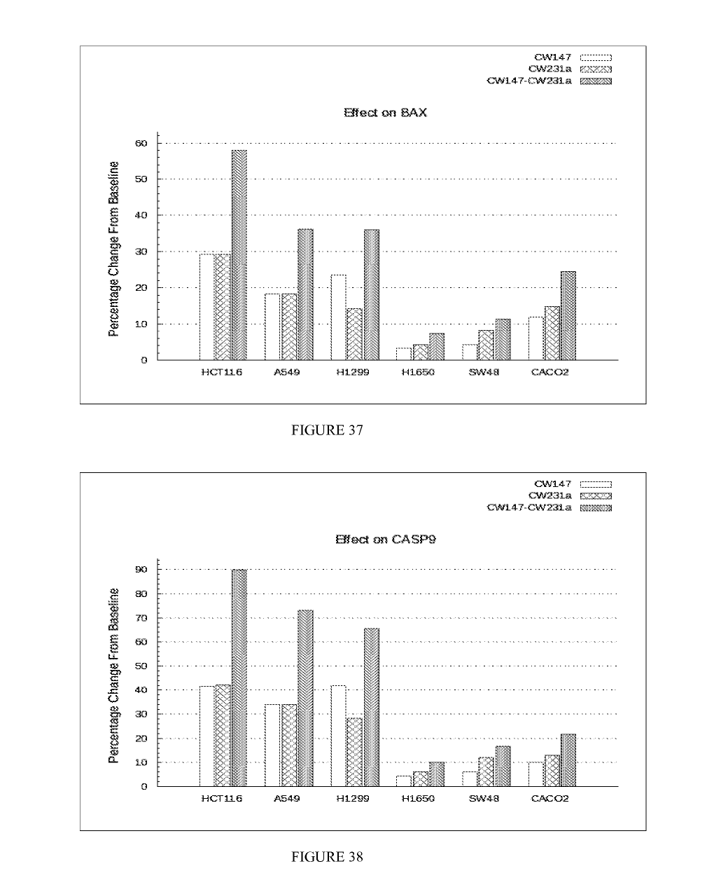

The predicted effect of ARQ-197 alone, Atorvastatin alone, or ARQ-197 and Atorvastatin in combination on levels of the apoptotic markers BAX (FIG. 37) and CASP9 (FIG. 38) was simulated using cells with (HCT116, A549, and H1299) or without (H1650, SW48, CaCo2) RAS mutations. When used in the combination, the compounds were used at their IC₂₅ concentrations. When administered alone, the compounds moderately affected BAX and CASP9 levels, whereas that effect was almost negligible in the cells without RAS mutations. BAX and CASP9 levels greatly increased when the compounds were used in combination, whereas that effect was only marginal in the cells without RAS mutations.

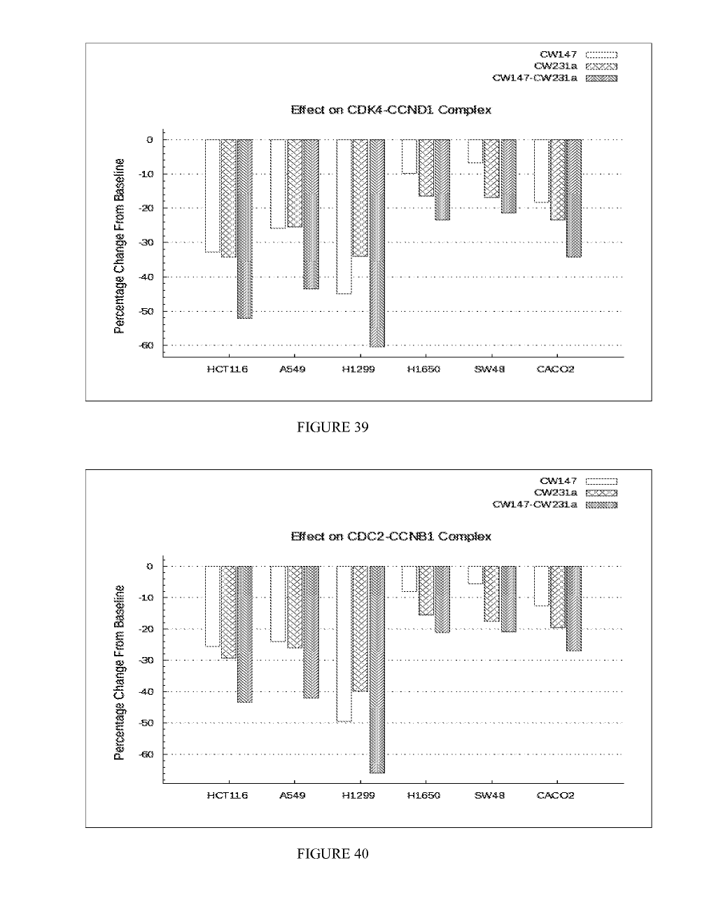

The predicted effect of ARQ-197 alone, Atorvastatin alone, or ARQ-197 and Atorvastatin in combination on levels of the proliferation markers CDK4-CCND1 (FIG. 39) and CDC2-CCNB1 (FIG. 40) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of the CDK4-CCND1 and CDC2-CCNB1 complexes, whereas that effect was almost negligible in the cells without RAS mutations. CDK4-CCND1 and CDC2-CCNB1 complex levels greatly decreased when the compounds were used in combination, whereas that effect was only marginal in the cells without RAS mutations.

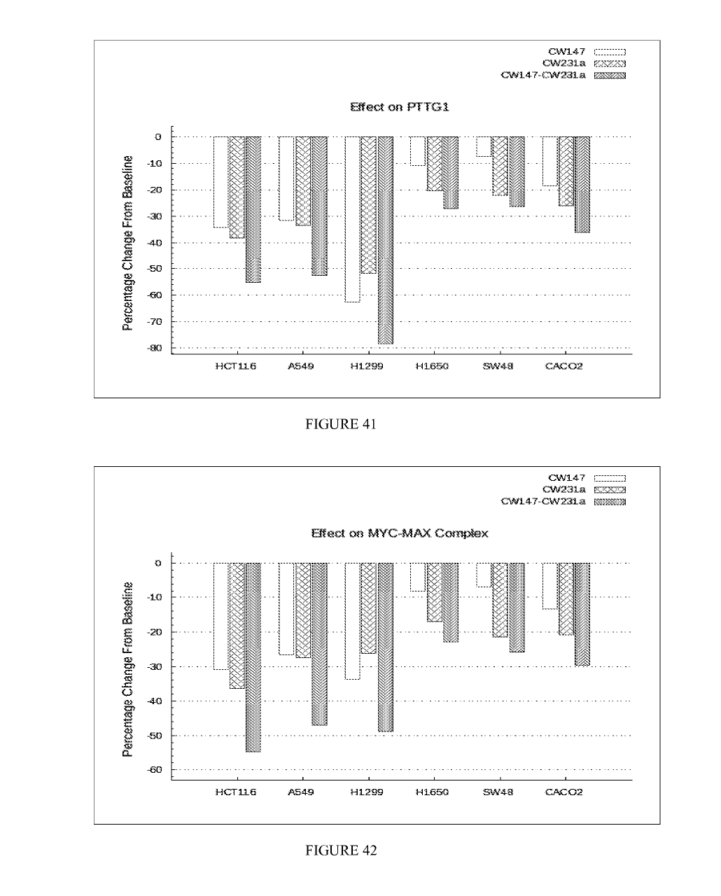

The predicted effect of ARQ-197 alone, Atorvastatin alone, or ARQ-197 and Atorvastatin in combination on levels of the cell growth markers PTTG1 (FIG. 41) and MYC-MAX (FIG. 42) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of PTTG1 and the MYC-MAX complex, whereas that effect was almost negligible in the cells without RAS mutations. PTTG1 and MYC-MAX levels greatly decreased when the compounds were used in combination, whereas that effect was only marginal in the cells without RAS mutations.

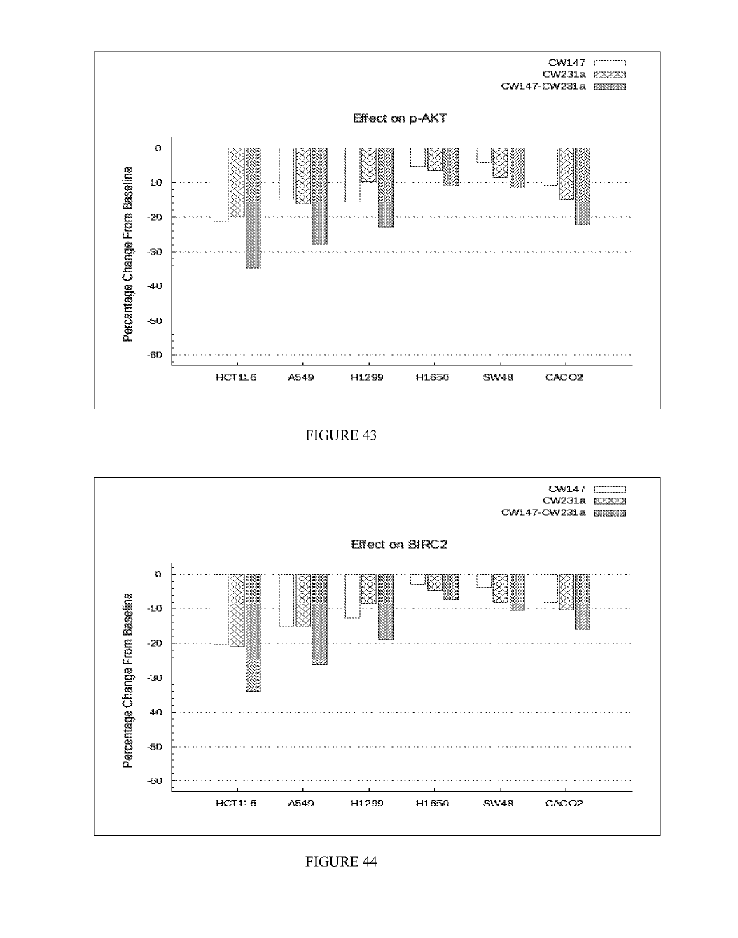

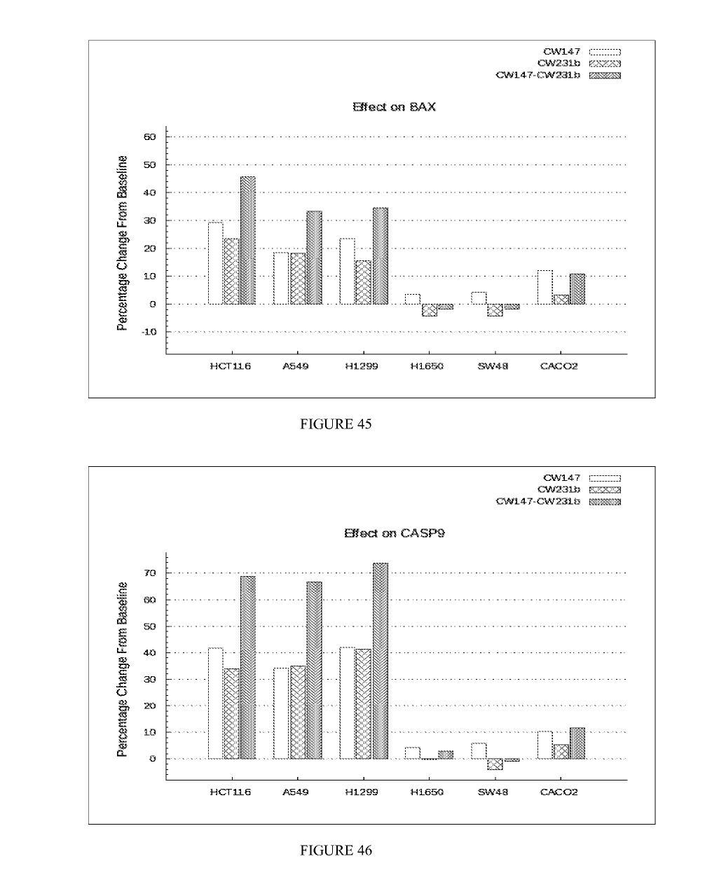

The predicted effect of ARQ-197 alone, Atorvastatin alone, or ARQ-197 and Atorvastatin in combination on levels of the cell survival markers p-AKT (FIG. 43) and BIRC2 (FIG. 44) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of p-AKT and BIRC2, whereas that effect was almost negligible in the cells without RAS mutations. p-AKT and BIRC2 levels greatly decreased when the compounds were used in combination, whereas that effect was only marginal in the cells without RAS mutations.

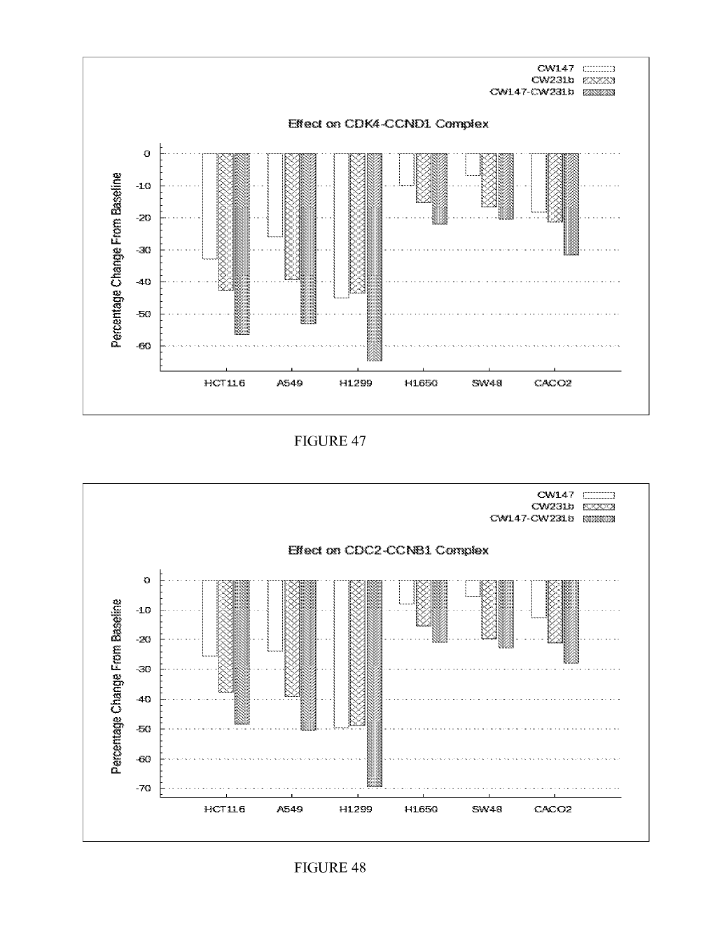

The predicted effect of ARQ-197 alone, BMS-303141 alone, or ARQ-197 and BMS-303141 in combination on levels of the apoptotic markers BAX (FIG. 45) and CASP9 (FIG. 46) was simulated using cells with (HCT116, A549, and H1299) or without (H1650, SW48, CaCo2) RAS mutations. When used in the combination, the compounds were used at their IC₂₅ concentrations. When administered alone, the compounds moderately affected BAX and CASP9 levels, whereas that effect was almost negligible in the cells without RAS mutations. BAX and CASP9 levels greatly increased when the compounds were used in combination, whereas that effect was only marginal in the cells without RAS mutations.

The predicted effect of ARQ-197 alone, BMS-303141 alone, or ARQ-197 and BMS-303141 in combination on levels of the proliferation markers CDK4-CCND1 (FIG. 47) and CDC2-CCNB1 (FIG. 48) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of the CDK4-CCND1 and CDC2-CCNB1 complexes, whereas that effect was almost negligible in the cells without RAS mutations. CDK4-CCND1 and CDC2-CCNB1 complex levels greatly decreased when the compounds were used in combination, whereas that effect was only marginal in the cells without RAS mutations.

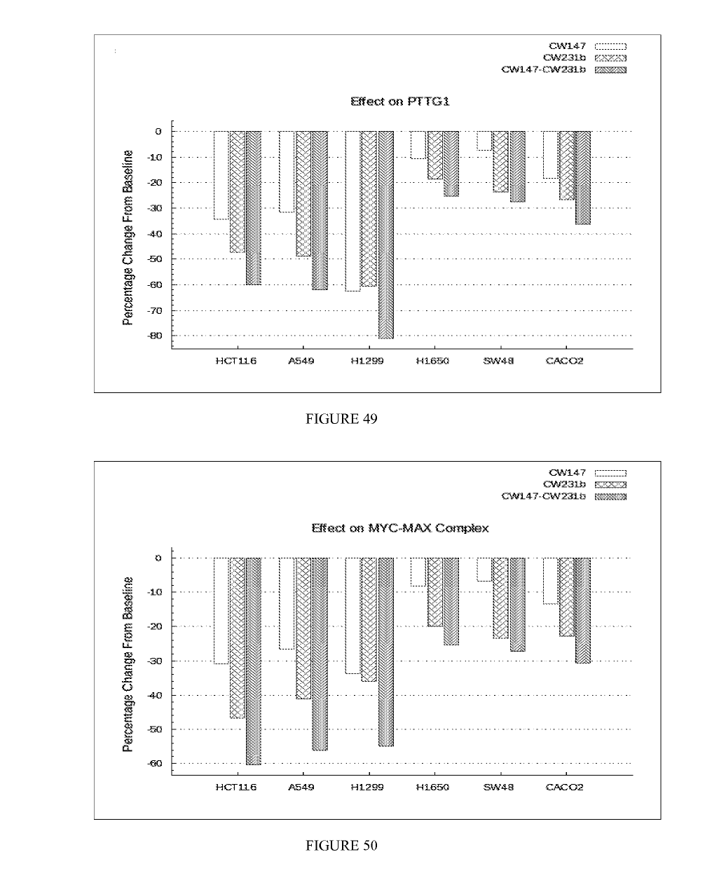

The predicted effect of ARQ-197 alone, BMS-303141 alone, or ARQ-197 and BMS-303141 in combination on levels of the cell growth markers PTTG1 (FIG. 49) and MYC-MAX (FIG. 50) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of PTTG1 and the MYC-MAX complex, whereas that effect was almost negligible in the cells without RAS mutations. PTTG1 and MYC-MAX levels greatly decreased when the compounds were used in combination, whereas that effect was only marginal in the cells without RAS mutations.

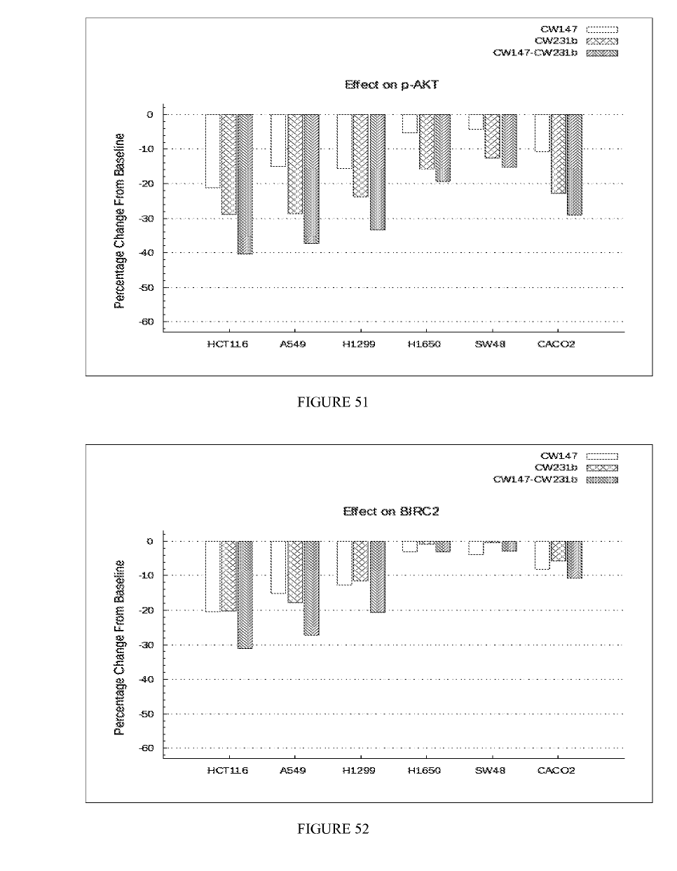

The predicted effect of ARQ-197 alone, BMS-303141 alone, or ARQ-197 and BMS-303141 in combination on levels of the cell survival markers p-AKT (FIG. 51) and BIRC2 (FIG. 52) was simulated using cells with (HCT116, A549, and H1299) or without (H1650, SW48, CaCo2) RAS mutations. When used in the combination, the compounds were used at their IC₂₅ concentrations. When administered alone, the compounds moderately affected levels of p-AKT and BIRC2, whereas that effect was almost negligible in the cells without RAS mutations. p-AKT and BIRC2 levels greatly decreased when the compounds were used in combination, whereas that effect was only marginal in the cells without RAS mutations.

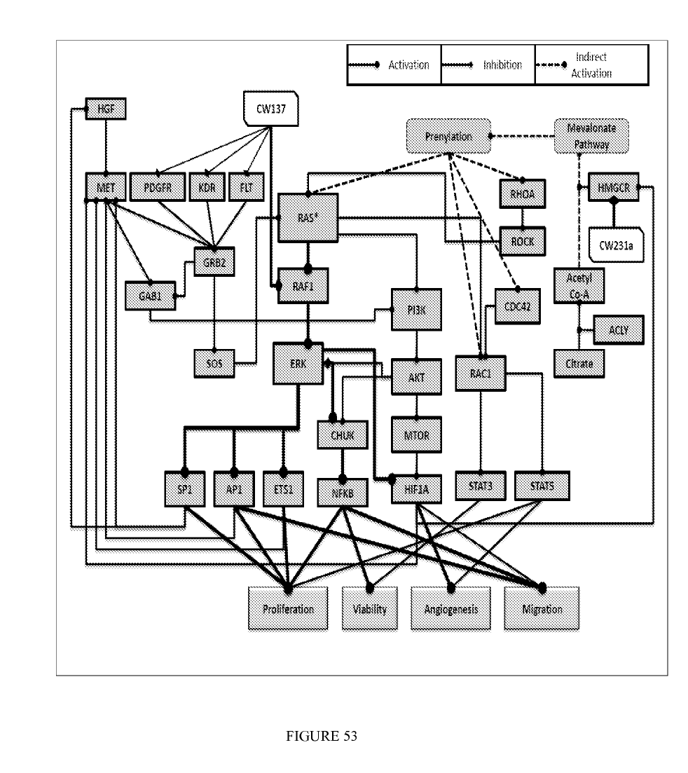

The scientific rationale for the effect of Sorafenib and Atorvastatin on key phenotypes of RAS-driven cancer is illustrated schematically in FIG. 53. The scheme shows that upon administration of Sorafenib, RAF is inhibited, leading to a block in the RAS-RAF-ERK signaling pathway. Further, administration of Atorvastatin causes inhibition of HMG-CoA reductase (HMGCR), thereby inhibiting the mevalonate pathway, ultimately preventing prenylation of RAS. Inhibition of the RAS-RAF-ERK signaling pathway and a decrease in the post-translational modifications of RAS can work to less the proliferation, angiogenesis, migration and increase in viability of tumor cells harboring mutations in RAS synergistically and therapeutically.

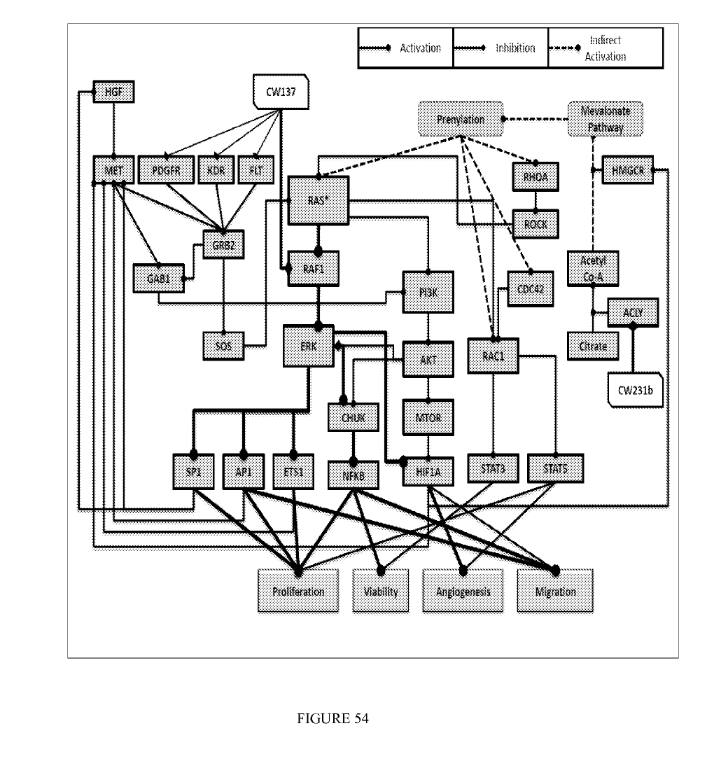

The scientific rationale for the effect of Sorafenib and BMS-303141 on key phenotypes of RAS-driven cancer is illustrated schematically in FIG. 54. The scheme shows that upon administration of Sorafenib, RAF is inhibited, leading to a block in the RAS-RAF-ERK signaling pathway. Further, administration of BMS-303141 causes inhibition of ATP citrate lyase (ACLY), thereby inhibiting the mevalonate pathway, ultimately preventing prenylation of RAS. Inhibition of the RAS-RAF-ERK signaling pathway and a decrease in the post-translational modifications of RAS can work to lessen the proliferation, angiogenesis, migration and increase in viability of tumor cells harboring mutations in RAS synergistically and therapeutically.

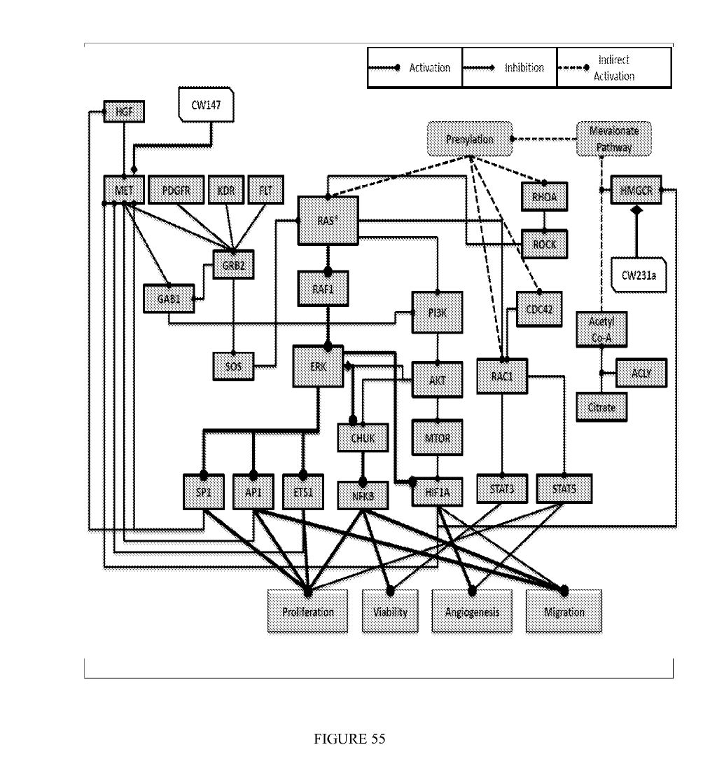

The scientific rationale for the effect of ARQ-197 and Atorvastatin on key phenotypes of RAS-driven cancer is illustrated schematically in FIG. 55. The scheme shows that upon administration of ARQ-197, MET is inhibited, leading to a block in the PI3K signaling pathway. Additionally, overexpressed MET is often found in tumors harboring RAS mutations as MET is a transcriptional target of some of the transcription factors activated by the RAS-RAF-ERK pathway. Administration of Atorvastatin causes inhibition of HMG-CoA reductase (HMGCR), thereby inhibiting the mevalonate pathway, ultimately preventing prenylation of RAS. Inhibition of MET-induced signaling and a decrease in the post-translational modifications of RAS can work to lessen the proliferation, angiogenesis, migration and increase in viability of tumor cells harboring mutations in RAS synergistically and therapeutically.

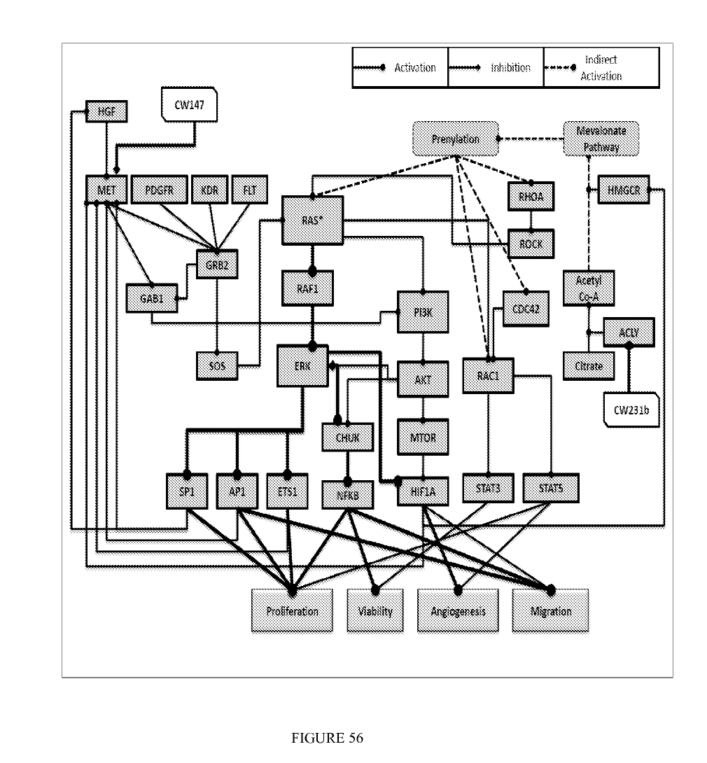

The scientific rationale for the effect of ARQ-197 and BMS-303141 on key phenotypes of RAS-driven cancer is illustrated schematically in FIG. 56. The scheme shows that upon administration of ARQ-197, MET is inhibited, leading to a block in the PI3K signaling pathway. Additionally, overexpressed MET is often found in tumors harboring RAS mutations as MET is a transcriptional target of some of the transcription factors activated by the RAS-RAF-ERK pathway. Administration of BMS-303141 causes inhibition of ATP citrate lyase (ACLY), thereby inhibiting the mevalonate pathway, ultimately preventing prenylation of RAS. Inhibition of MET-induced signaling and a decrease in the post-translational modifications of RAS can work to lessen the proliferation, angiogenesis, migration and increase in viability of tumor cells harboring mutations in RAS synergistically and therapeutically.

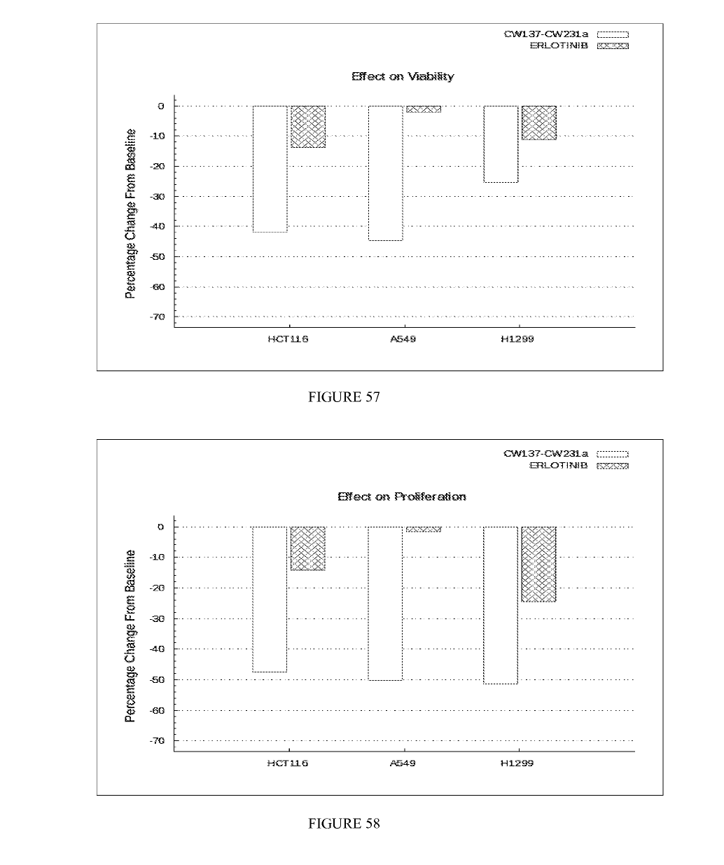

FIG. 57 illustrates the predicted effect of Sorafenib and Atorvastatin compared to Erlotinib, an anti-EGFR drug that can be used in the treatment of NSCLC, on the viability of mutant RAS cell lines. The combination of Sorafenib and Atorvastatin provided a greater decrease of viability of mutant RAS cell lines as compared to Erlotinib, which only marginally decreased viability.

FIG. 58 illustrates the predicted effect of Sorafenib and Atorvastatin compared to Erlotinib, an anti-EGFR drug that can be used in the treatment of NSCLC, on the proliferation of mutant RAS cell lines. The combination of Sorafenib and Atorvastatin provided a greater decrease of proliferation of mutant RAS cell lines as compared to Erlotinib, which only marginally decreased proliferation.

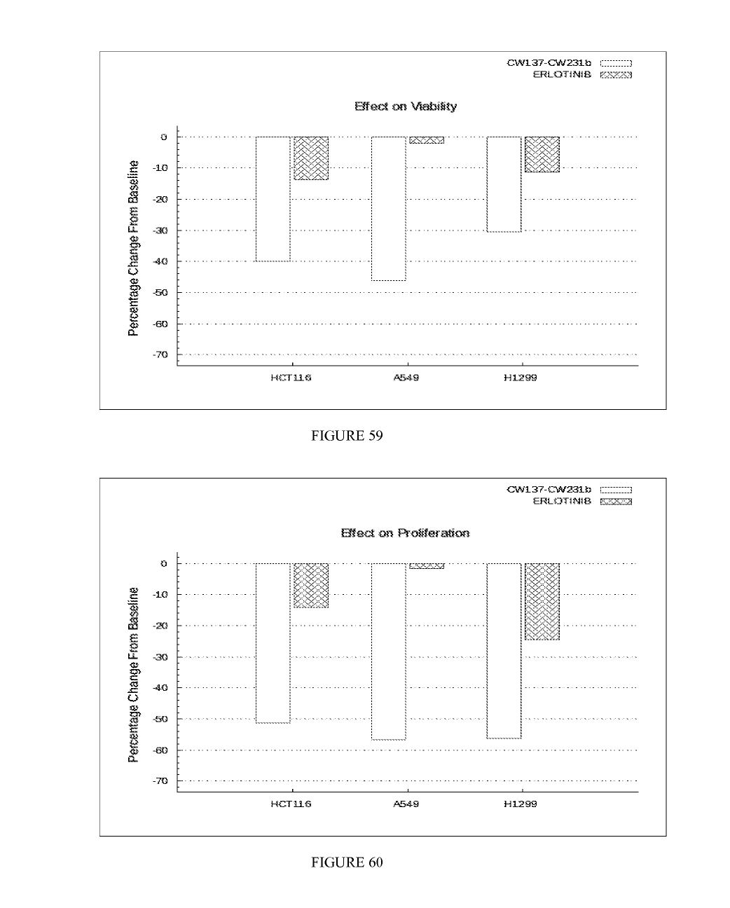

FIG. 59 illustrates the predicted effect of Sorafenib and BMS-303141 compared to Erlotinib, an anti-EGFR drug that can be used in the treatment of NSCLC, on the viability of mutant RAS cell lines. The combination of Sorafenib and BMS-303141 provided a greater decrease of viability of mutant RAS cell lines as compared to Erlotinib, which only marginally decreased viability.

FIG. 60 illustrates the predicted effect of Sorafenib and BMS-303141 compared to Erlotinib, an anti-EGFR drug that can be used in the treatment of NSCLC, on the proliferation of mutant RAS cell lines. The combination of Sorafenib and BMS-303141 provided a greater decrease in proliferation of mutant RAS cell lines as compared to Erlotinib, which only marginally decreased proliferation.

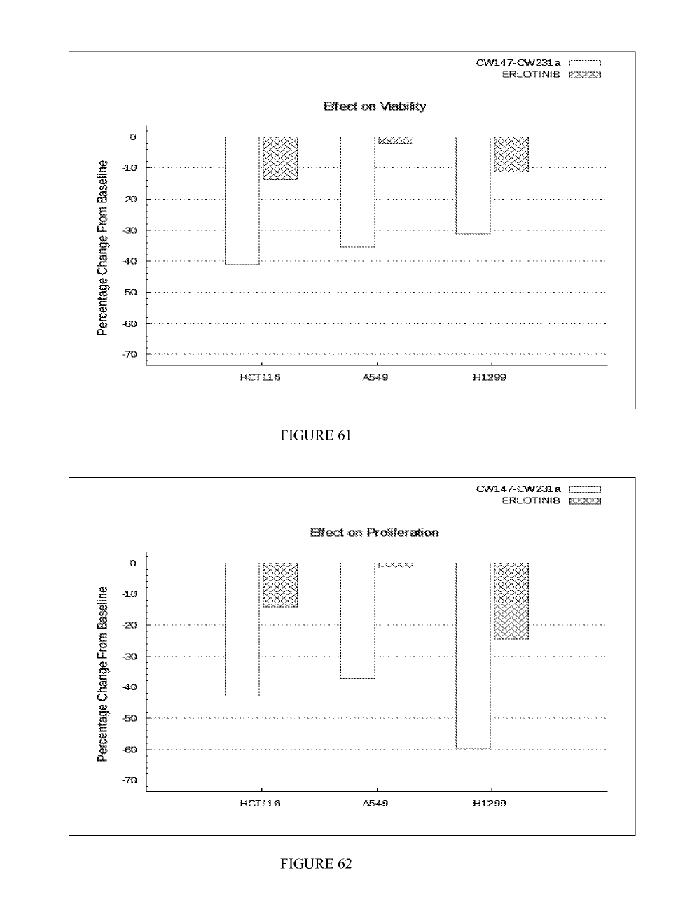

FIG. 61 illustrates the predicted effect of ARQ-197 and Atorvastatin compared to Erlotinib, an anti-EGFR drug that can be used in the treatment of NSCLC, on the viability of mutant RAS cell lines. The combination of ARQ-197 and Atorvastatin provided a greater decrease of viability of mutant RAS cell lines as compared to Erlotinib, which only marginally decreased viability.

FIG. 62 illustrates the predicted effect of ARQ-197 and Atorvastatin compared to Erlotinib, an anti-EGFR drug that can be used in the treatment of NSCLC, on the proliferation of mutant RAS cell lines. The combination of ARQ-197 and Atorvastatin provided a greater decrease in proliferation of mutant RAS cell lines as compared to Erlotinib, which only marginally decreased proliferation.

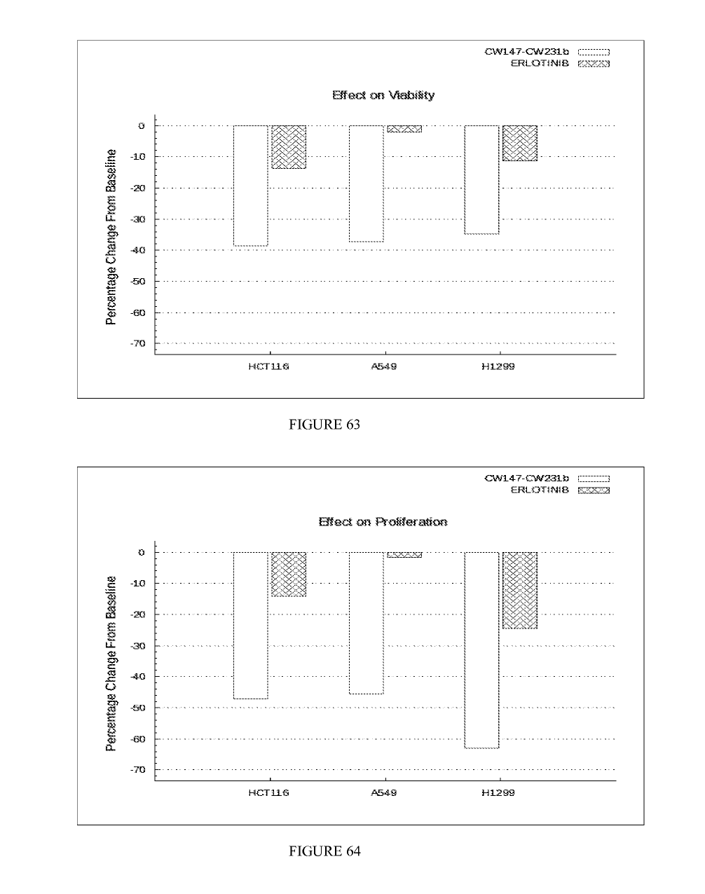

FIG. 63 illustrates the predicted effect of ARQ-197 and BMS-303141 compared to Erlotinib, an anti-EGFR drug that can be used in the treatment of NSCLC, on the viability of mutant RAS cell lines. The combination of ARQ-197 and BMS-303141 provided a greater decrease of viability of mutant RAS cell lines as compared to Erlotinib, which only marginally decreased viability.

FIG. 64 illustrates the predicted effect of ARQ-197 and BMS-303141 compared to Erlotinib, an anti-EGFR drug that can be used in the treatment of NSCLC, on the proliferation of mutant RAS cell lines. The combination of ARQ-197 and BMS-303141 provided a greater decrease in proliferation of mutant RAS cell lines as compared to Erlotinib, which only marginally decreased proliferation.

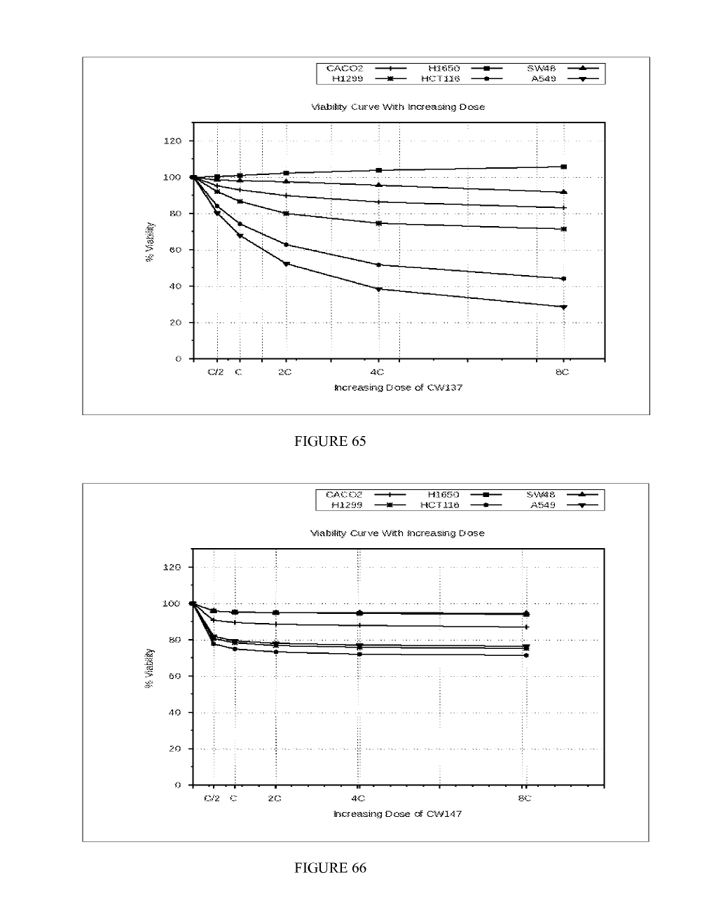

FIG. 65 depicts a predicted dose response curve for Sorafenib on the viability of cells with (HCT116, A549, and H1299) or without (H1650, SW48, CaCo2) RAS mutations. The viability of the mutant RAS cell lines continued to decrease with increasing amounts of Sorafenib, while the cell lines without RAS mutations were minimally affected by increasing amounts of Sorafenib.

FIG. 66 depicts a predicted dose response curve for ARQ-197 on the viability of cells with (HCT116, A549, and H1299) or without (H1650, SW48, CaCo2) RAS mutations. The viability of the mutant RAS cell lines decreased most significantly when ARQ-197 was initially administered and continued to decrease slightly until the dosage reached four times the initial concentration. At this point, the viability plateaued even with increasing amounts of ARQ-197. A similar, but less significant, trend was seen in the cell lines without RAS mutations.

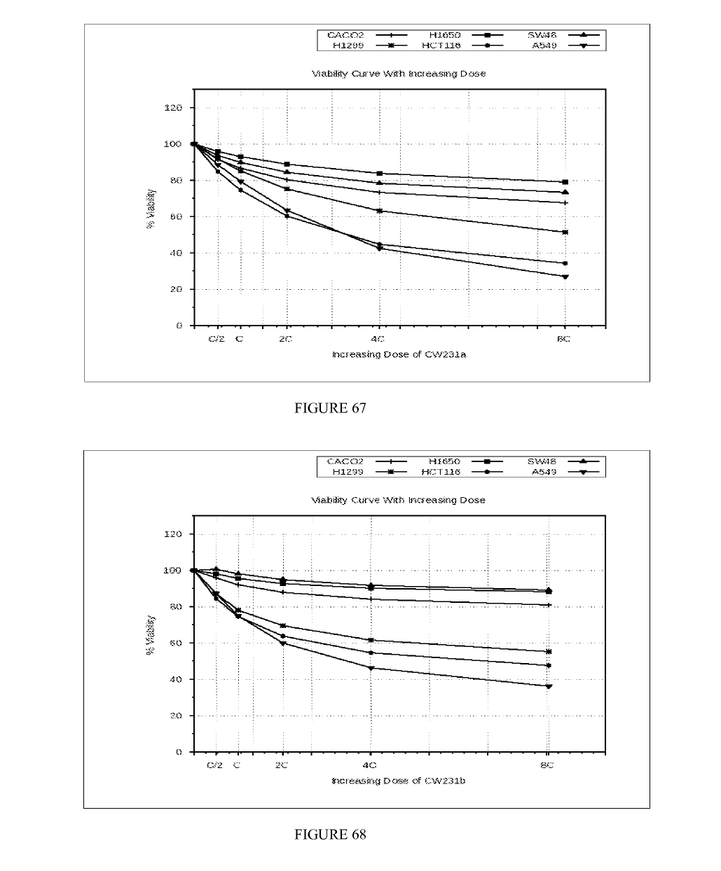

FIG. 67 depicts a predicted dose response curve for Atorvastatin on the viability of cells with (HCT116, A549, and H1299) or without (H1650, SW48, CaCo2) RAS mutations. The viability of the mutant RAS cell lines continued to decrease with increasing amounts of Atorvastatin. A similar, but less significant, trend was seen in the cell lines without RAS mutations.

FIG. 68 depicts a predicted dose response curve for BMS-303141 on the viability of cells with (HCT116, A549, and H1299) or without (H1650, SW48, CaCo2) RAS mutations. The viability of the mutant RAS cell lines continued to decrease with increasing amounts of BMS-303141, while the cell lines without RAS mutations were minimally affected by increasing amounts of BMS-303141.

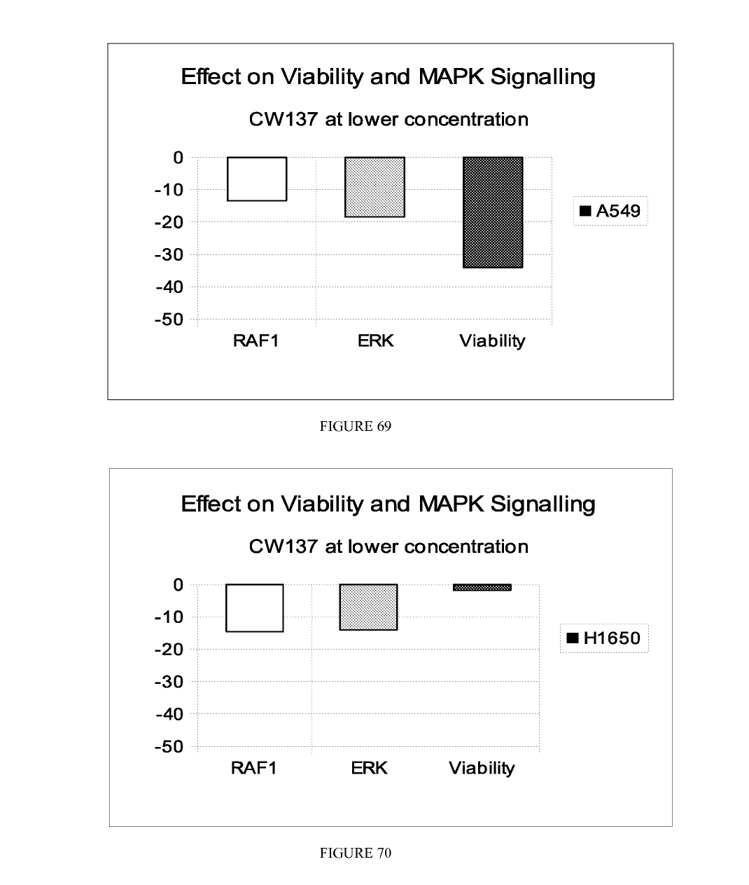

The specificity of Sorafenib toward mutant RAS cell lines is illustrated by the predicted effect that a low concentration of Sorafenib has on RAF1 and ERK levels in A549 (FIG. 69) as compared to H1650 (FIG. 70) cells. In A549 cells, Sorafenib was able to decrease RAF1 and ERK levels by about 12% and about 19%, respectively. Sorafenib can also decrease the viability of A549 cells by almost 35%. In the RAS wild type cells (FIG. 70), Sorafenib decreased RAF1 and ERK by about 15%, but had a negligible effect on the viability of these cells.

### EXAMPLES

Non-limiting examples are presented below. As used in the figures of the present disclosure and references to the same, CW137 is exemplified by Sorafenib (RAF inhibitor); CW147 is exemplified by ARQ-197 (c-MET inhibitor, examples 14-25) or Crizotinib (c-MET inhibitor, examples 26 and 27); CW231a is exemplified by Atorvastatin (HMG-CoA reductase inhibitor); CW231b is exemplified by BMS-303141 (ATP Citrate Lyase Inhibitor); and CW231c is exemplified by Tipifarnib (Farnesyl transferase inhibitor).

TABLE 4 briefly illustrates the description of the examples and their respective figures.

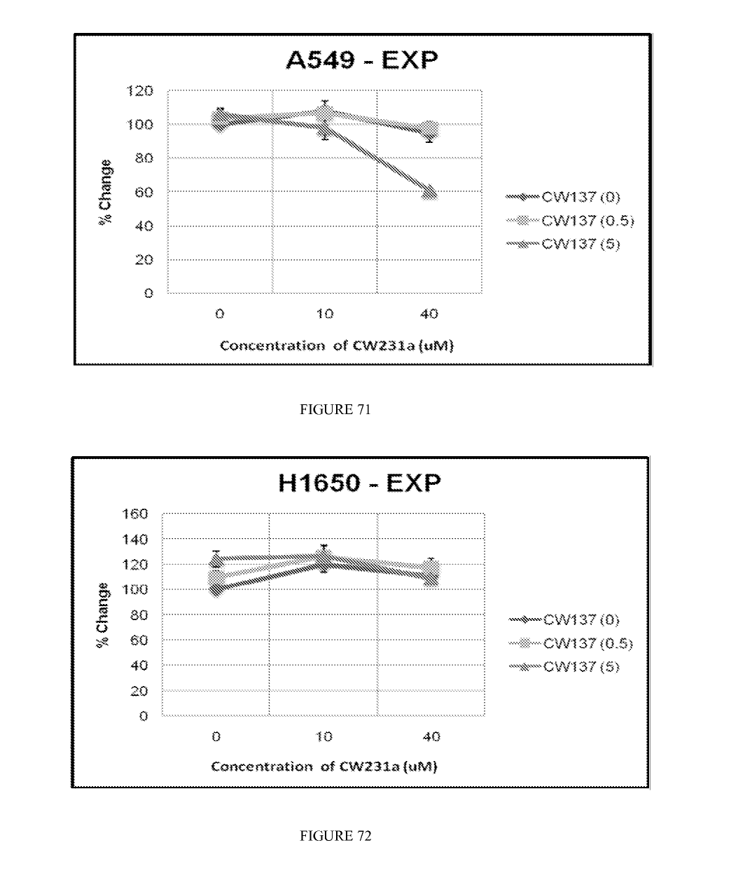

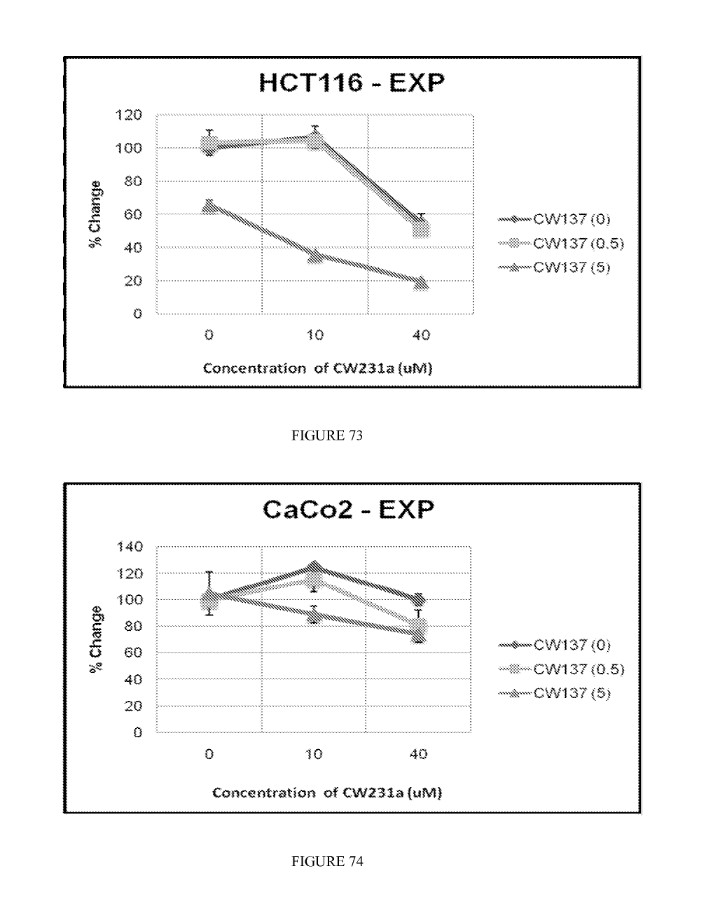

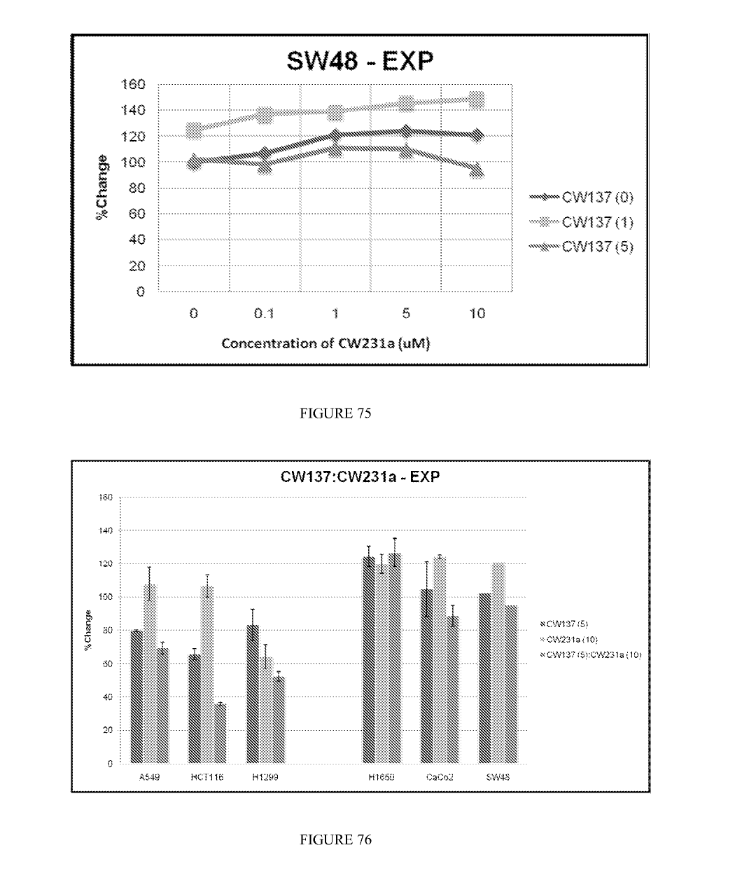

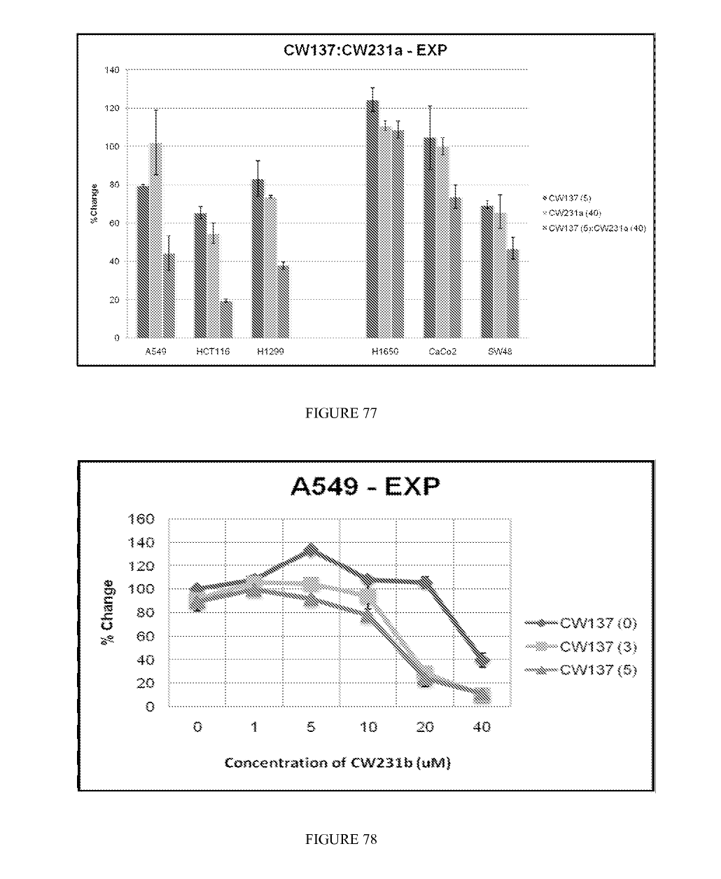

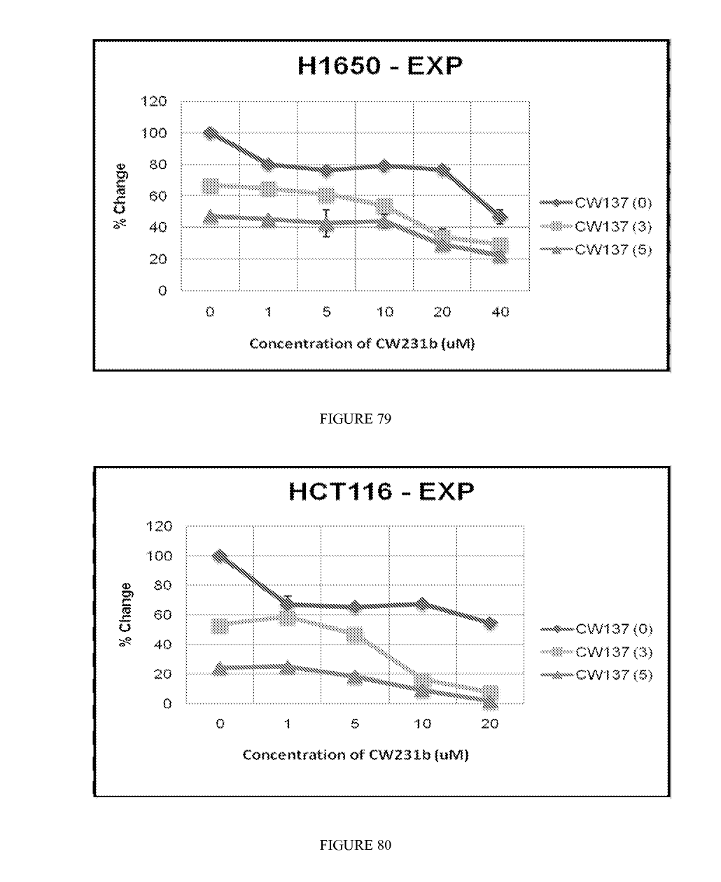

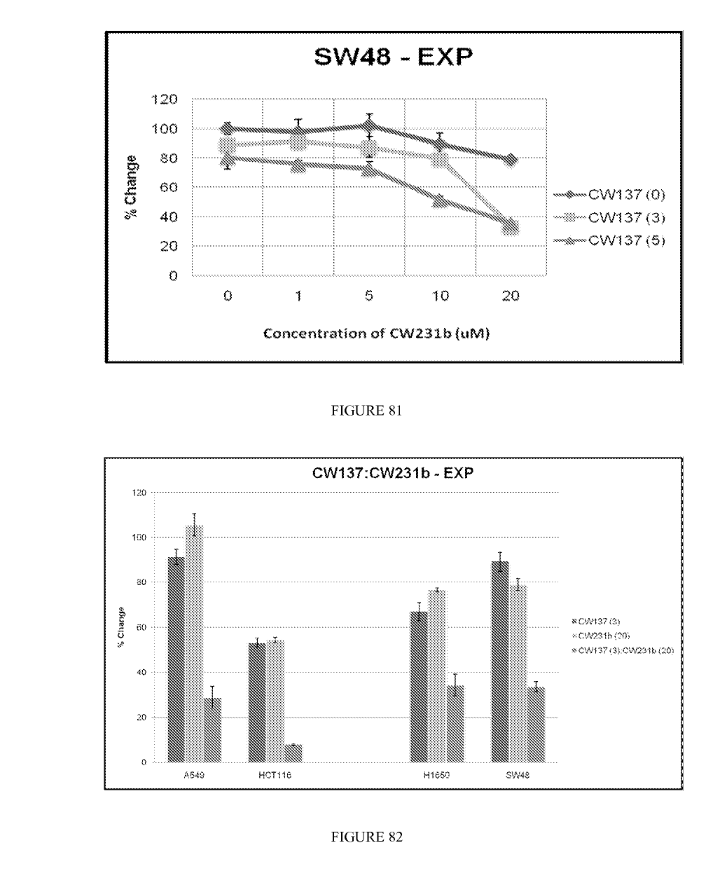

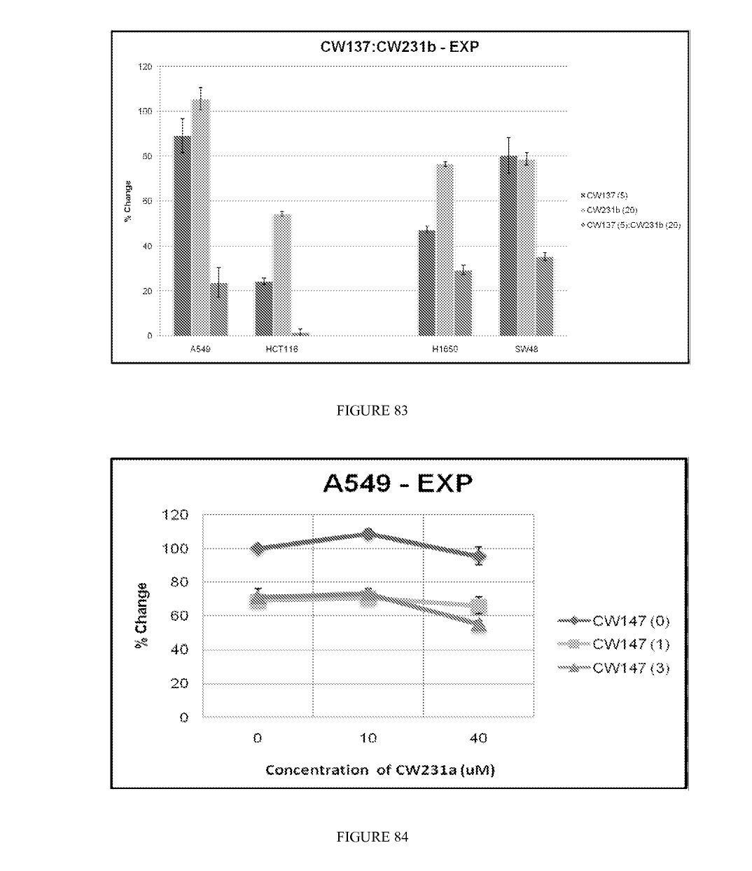

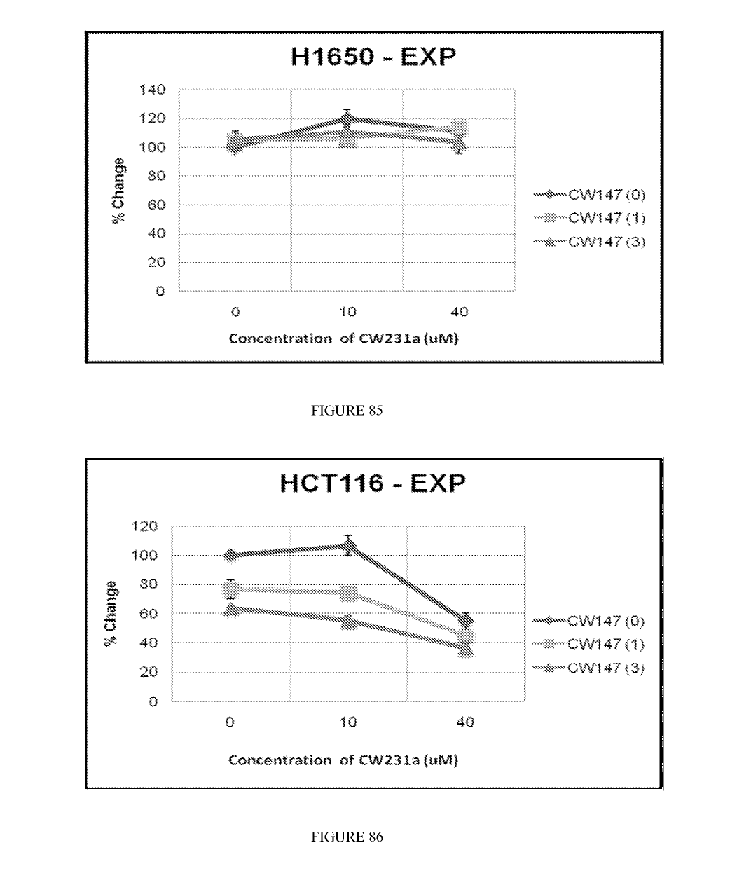

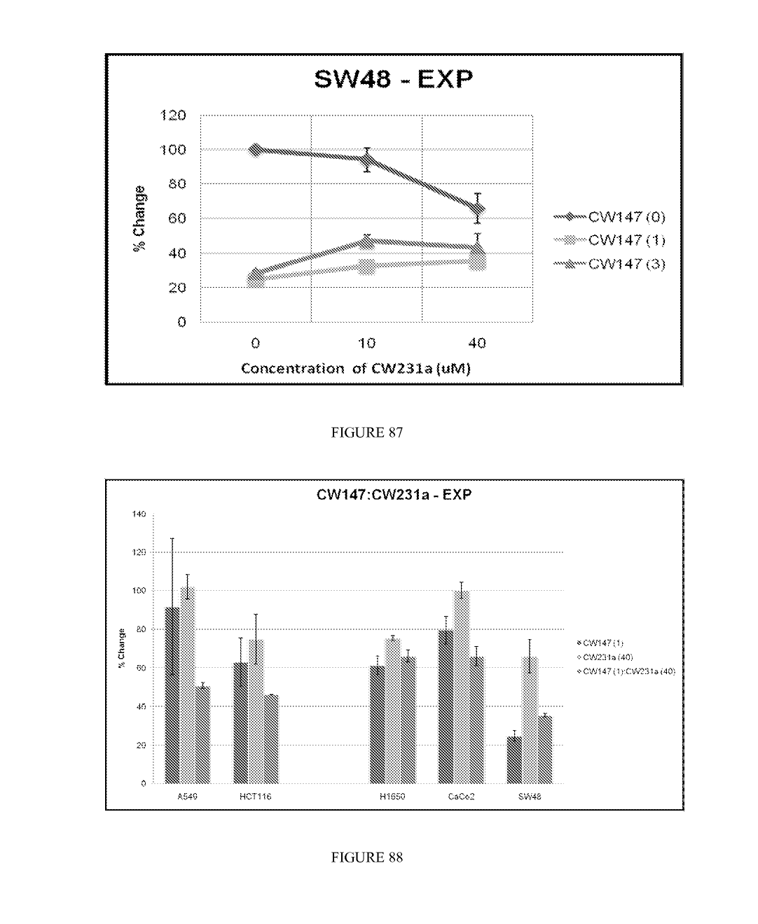

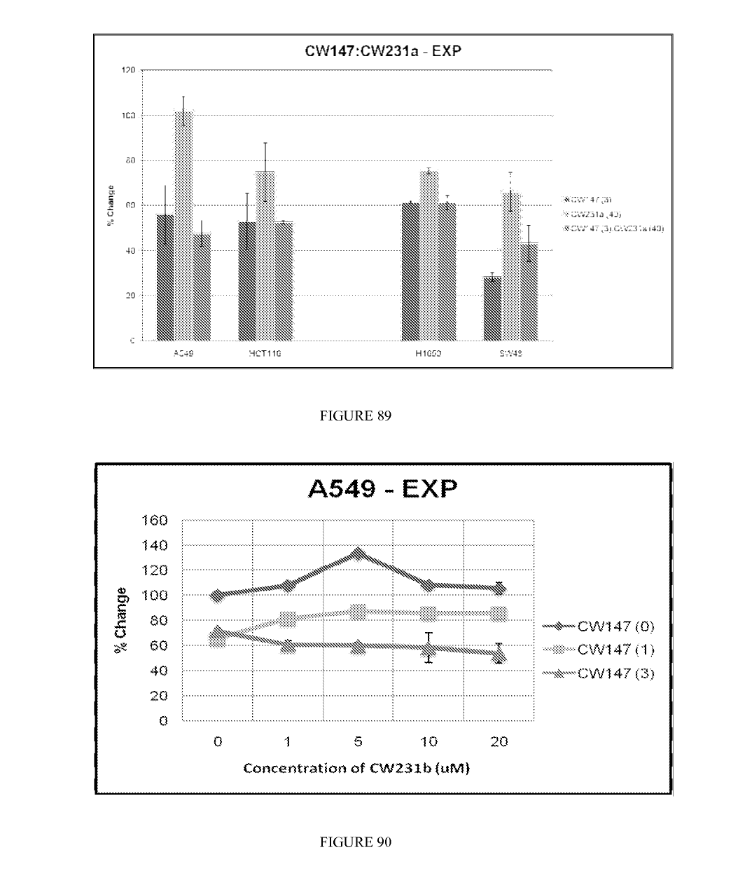

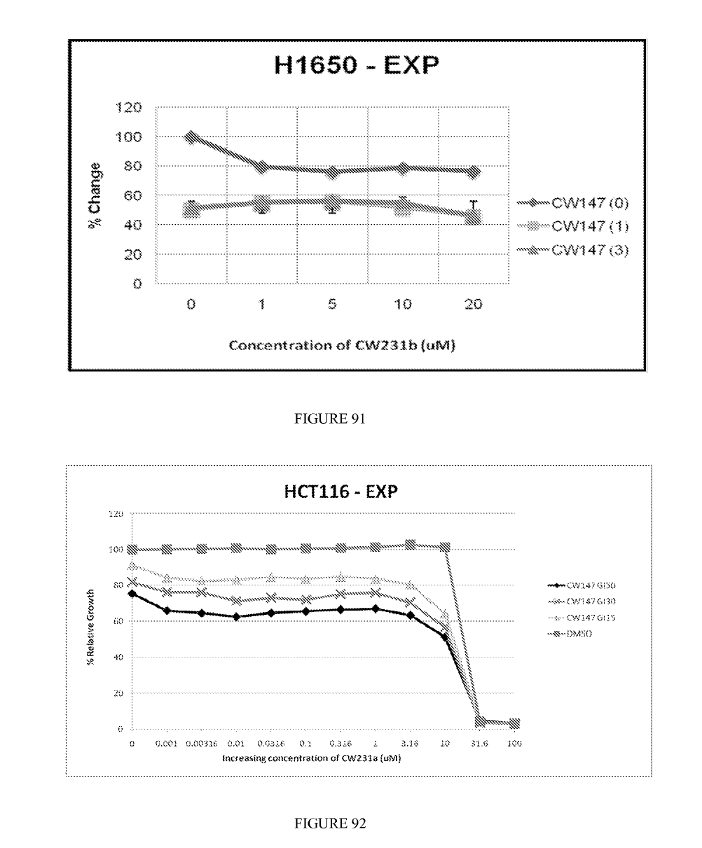

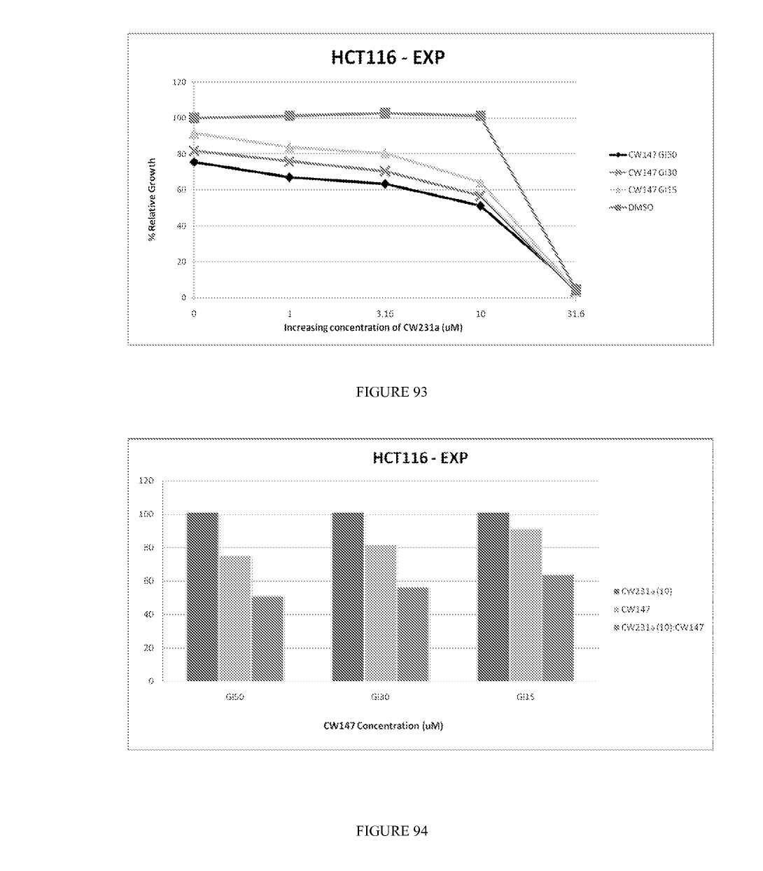

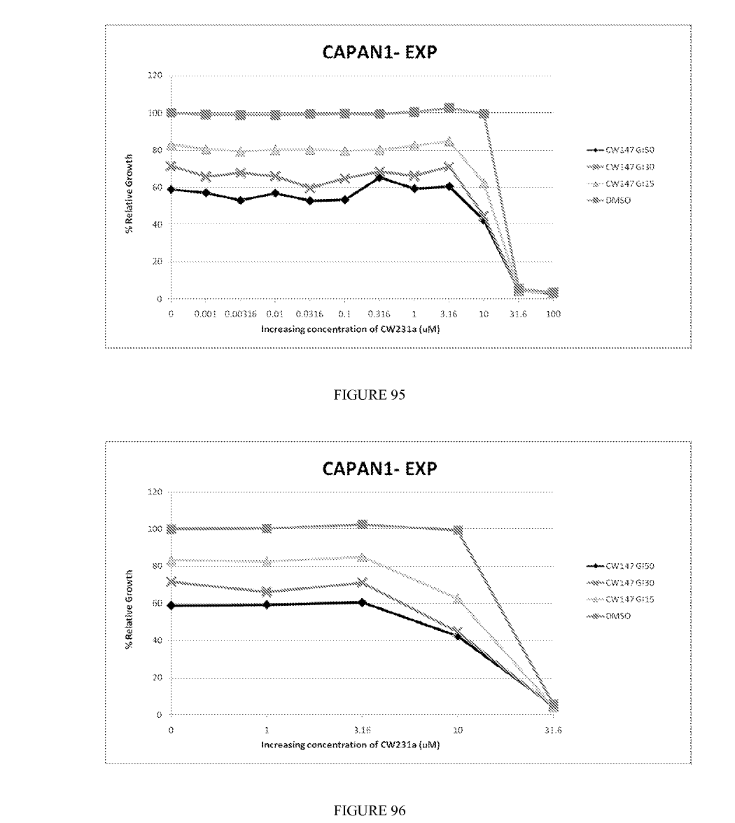

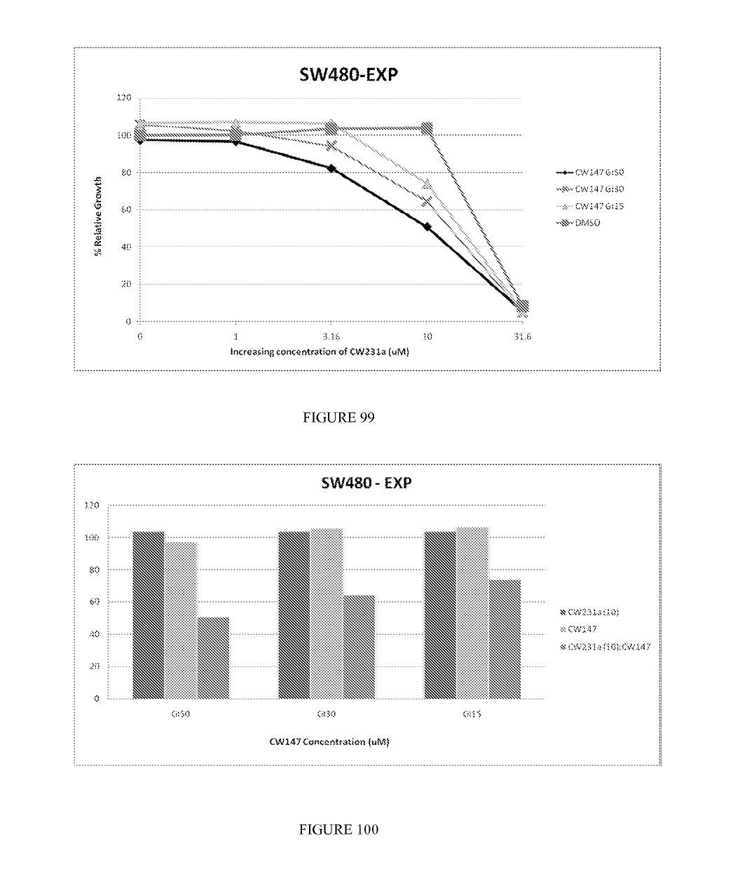

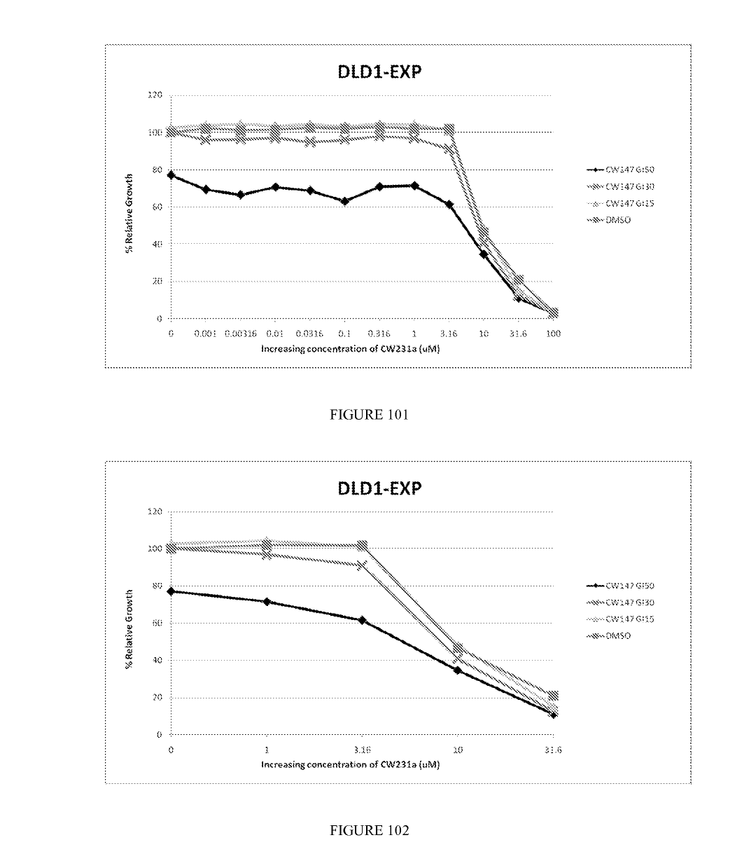

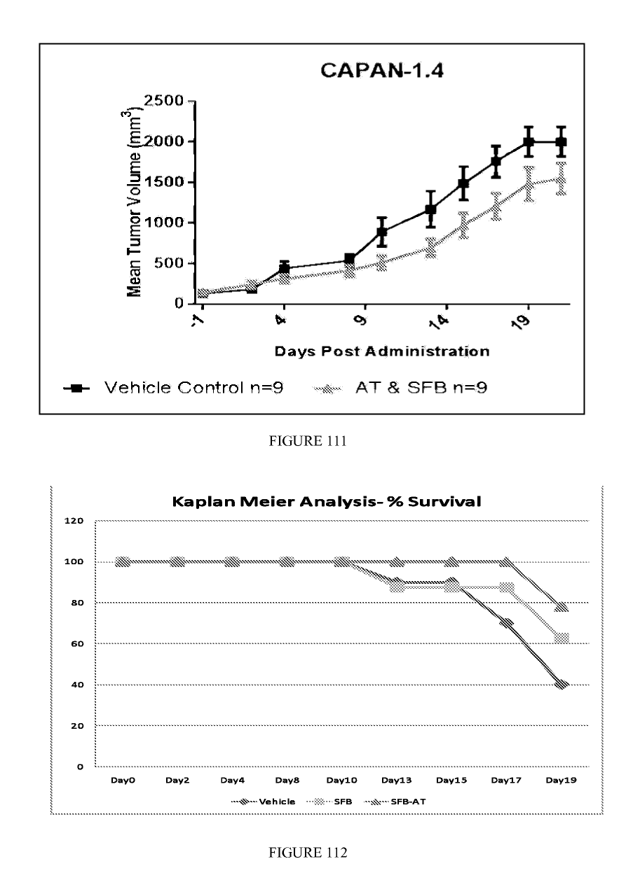

TABLE 4

| Example | Description of the Example | FIGS. |
|---|---|---|
| Example 1 | Effect of CW137-CW231a in KRAS mutant A549 human | FIG. 71 |
| | lung cancer cell line | |
| Example 2 | Effect of CW137-CW231a in KRAS wild type H1650 | FIG. 72 |
| | human lung cancer cell line | |
| Example 3 | Effect of CW137-CW231a in KRAS mutant HCT116 | FIG. 73 |
| | human colon cancer cell line | |
| Example 4 | Effect of CW137-CW231a in KRAS wild type CaCo-2 | FIG. 74 |
| | human colon cancer cell line | |
| Example 5 | Effect of CW137-CW231a in KRAS wild type SW48 | FIG. 75 |
| | human colon cancer cell line | |
| Example 6 | Summary plot of the effect of CW137-CW231a | FIG. 76 |
| | combined at 5 μM CW137 and 10 μM CW231a, | |
| | compared between RAS mutant and RAS wild type | |
| | cell lines | |
| Example 7 | Summary plot of the effect of CW137-CW231a | FIG. 77 |
| | combined at 5 μM CW137 and 40 μM CW231a, | |
| | compared between RAS mutant and RAS wild type | |
| | cell lines | |
| Example 8 | Effect of CW137-CW231b in KRAS mutant A549 | FIG. 78 |
| | human lung cancer cell line | |
| Example 9 | Effect of CW137-CW231b in KRAS wild type H1650 | FIG. 79 |
| | human lung cancer cell line | |
| Example 10 | Effect of CW137-CW231b in KRAS mutant HCT116 | FIG. 80 |
| | human colon cancer cell line | |
| Example 11 | Effect of CW137-CW231b in KRAS wild type SW48 | FIG. 81 |
| | human colon cancer cell line | |
| Example 12 | Summary plot of the effect of CW137-CW231b | FIG. 82 |
| | combined at 3 μM CW137 and 20 μM CW231b, | |
| | compared between RAS mutant and RAS wild type | |
| | cell lines | |
| Example 13 | Summary plot of the effect of CW137-CW231b | FIG. 83 |
| | combined at 5 μM CW137 and 20 μM CW231b, | |
| | compared between RAS mutant and RAS wild type | |
| | cell lines | |
| Example 14 | Effect of CW147-CW231a in KRAS mutant A549 | FIG. 84 |
| | human lung cancer cell line | |
| Example 15 | Effect of CW147-CW231a in KRAS wild type H1650 | FIG. 85 |
| | human lung cancer cell line | |
| Example 16 | Effect of CW147-CW231a in KRAS mutant HCT116 | FIG. 86 |
| | human colon cancer cell line | |
| Example 17 | Effect of CW147-CW231a in KRAS wild type SW48 | FIG. 87 |
| | human colon cancer cell line | |
| Example 18 | Summary plot of the effect of CW147-CW231a | FIG. 88 |
| | combined at 1 μM CW147 and 40 μM CW231a, | |
| | compared between RAS mutant and RAS wild type | |
| | cell lines | |
| Example 19 | Summary plot of the effect of CW147-CW231a | FIG. 89 |
| | combined at 3 μM CW147 and 40 μM CW231a, | |
| | compared between RAS mutant and RAS wild type | |
| | cell lines | |
| Example 20 | Effect of CW147-CW231b in KRAS mutant A549 | FIG. 90 |
| | human lung cancer cell line | |
| Example 21 | Effect of CW147-CW231b in KRAS wild type H1650 | FIG. 91 |
| | human lung cancer cell line | |
| Example 22 | Effect of CW147-CW231a in KRAS mutant HCT116 | FIGS. 92, 93, |
| | human colon cancer cell line | 94 |
| Example 23 | Effect of CW147-CW231a in KRAS mutant CAPAN1 | FIGS. 95, 96, |
| | human pancreatic cancer cell line | 97 |
| Example 24 | Effect of CW147-CW231a in KRAS mutant SW480 | FIGS. 98, 99, |
| | human colon cancer cell line | 100 |
| Example 25 | Effect of CW147-CW231a in KRAS mutant DLD1 | FIGS. 101, |
| | human colon cancer cell line | 102, 103 |
| Example 26 | Effect of CW147-CW231a in KRAS mutant HCT116 | FIGS. 104, |
| | human colon cancer cell line | 105 |
| Example 27 | Effect of CW147-CW231c in KRAS mutant HCT116 | FIGS. 106, |
| | human colon cancer cell line | 107 |
| Example 28 | Effect of CW137-CW231a in mouse xenograft | FIGS. 108, |
| | model of KRAS mutant HCT116 human colon cancer | 109, 110 |
| | cell line | |
| Example 29 | Effect of CW137-CW231a in mouse xenograft model | FIGS. 111, |
| | of KRAS mutant CAPAN1 human pancreatic cancer | 112, 113 |
| | cell line | |

### Example 1

### Effect of Sorafenib-Atorvastatin on the A549 [RAS Mutant] Human Lung Cancer Cell Line

The A549 human lung cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (3,000 cells/well; passage 6). DMSO, digitoxin, and the compounds (Sorafenib and Atorvastatin at a concentration of about 0 μM to about 5 μM and about 0 μM to about 40 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 71 illustrates that when the cells were incubated with Sorafenib or Atorvastatin individually, a negligible decrease in A549 cell viability was observed. Increasing doses of Atorvastatin alone did not show any effect; however, combination with 5 μM Sorafenib, provided a significant and synergistic decrease in viability. FIG. 77 shows that the relative growth percentage of the cells with 5 μM Sorafenib was about 80% and with 40 μM Atorvastatin, about 102%. Upon incubating the cells with both Sorafenib (5 μM) and Atorvastatin (40 μM), a synergistic effect was observed with a relative growth of about 45%, which is about a 55% reduction in cell viability.

### Example 2

### Effect of Sorafenib-Atorvastatin on the H1650 [RAS Wild Type] Human Lung Cancer Cell Line

The H1650 human lung cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (5,000 cells/well; passage 6). DMSO, digitoxin, and the compounds (Sorafenib and Atorvastatin at a concentration of about 0 μM to about 5 μM and about 0 μM to about 40 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 72 illustrates that when the cells were incubated with two drugs, Sorafenib and Atorvastatin individually or in combination, a negligible decrease in H1650 cell viability was observed. Furthermore, FIG. 77 illustrates that the relative growth percentage of the cells with 5 μM Sorafenib was about 124% and with 40 μM Atorvastatin, about 110%. Upon incubating the cells with both Sorafenib (5 μM) and Atorvastatin (40 μM) a negligible effect on cell viability was observed with a relative growth of about 108%.

### Example 3

### Effect of Sorafenib-Atorvastatin on the HCT116 [RAS Mutant] Human Colon Cancer Cell Line

The HCT116 human colon cancer cell line was procured from the ATCC (American Type Culture Collection, Manassas, Va.). The cells were resuspended in media containing 5% FBS (Gibco) and 4× Gentamicin followed by transferring about 100 μl of the resuspension to each well in an assay plate (3,000 cells/well; passage 11). DMSO, digitoxin, and the compounds (Sorafenib and Atorvastatin at a concentration of about 0 μM to about 5 μM and about 0 μM to about 40 μM, respectively) were serially diluted in assay media. 100 μl of the diluted sample was added to each well of the assay plate containing the resuspended cells. The final assay volume of each well was about 200 μl containing 5% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 73 illustrates that when the HCT116 cells were incubated with Sorafenib and Atorvastatin individually, an insignificant decrease in HCT116 cell viability was observed. A minor reduction in viability was seen with the highest dose of Atorvastatin, which was further enhanced when combined with Sorafenib at 5 μM. Further, FIG. 77 illustrates that the relative growth percentage of the cells with 5 μM Sorafenib was about 65% and with 40 μM Atorvastatin, about 54%. Upon incubating the cells with both Sorafenib (5 μM) and Atorvastatin (40 μM), a synergistic effect was observed with a relative growth of about 19%, which is about an 80% reduction in cell viability.

### Example 4

### Effect of Sorafenib-Atorvastatin on the CaCo-2 [RAS Wild Type] Human Colon Cancer Cell Line

The CaCo-2 human colon cancer cell line was procured from ATCC (American Type Culture Collection, Manassas, Va.). The cells were resuspended in media containing 10% FBS (Gibco) and 4× Gentamicin followed by transferring about 100 μl of the resuspension to each well in an assay plate (5,400 cells/well; passage 5). DMSO, digitoxin, and the compounds (Sorafenib and Atorvastatin at a concentration of about 0 μM to about 5 μM and about 0 μM to about 40 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 74 illustrates that when the CaCo-2 cells were incubated with Sorafenib and Atorvastatin individually or in combination, a negligible effect on cell viability was observed. Further, FIG. 77 illustrates that the relative growth percentage of the cells with 5 μM Sorafenib was about 104% and with 40 μM Atorvastatin, about 100%. Upon incubating the cells with both Sorafenib (5 μM) and Atorvastatin (40 μM) a minor effect on cell viability was observed with a relative growth of about 73%.

### Example 5

### Effect of Sorafenib-Atorvastatin on the SW48 [RAS Wild Type] Human Colon Cancer Cell Line

The SW48 human colon cancer cell line was procured from ATCC (American Type Culture Collection, Manassas, Va.). The cells were resuspended in media containing 10% FBS (Gibco) and 4× Gentamicin followed by transferring about 100 μl of the resuspension to each well in an assay plate (5,300 cells/well; passage 3). DMSO, digitoxin, and the compounds (Sorafenib and Atorvastatin at a concentration of about 0 μM to about 5 μM and about 0 μM to about 10 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin and drugs., The assay plate was incubated at 37° C. for ab out 47 hours followed by the addition of 20 μl of CellTiter 96 Aqueous One Solution Reagent (Promega) to each well. The absorbance of the sample was read at 490 nm.

FIG. 75 illustrates that when the SW48 cells were incubated with Sorafenib and Atorvastatin individually or in combination, a negligible effect on cell viability was observed. Further, FIG. 76 illustrates that the relative growth percentage of the cells with 5 μM Sorafenib was about 102% and with 40 μM Atorvastatin, about 121%. Upon incubating the cells with both Sorafenib (5 μM) and Atorvastatin (40 μM) a negligible effect on cell viability was observed with a relative growth of about 95%.

### Example 6

### Effect of Sorafenib-Atorvastatin (Sorafenib, 5 μM, and Atorvastatin, 10 μM) Compared Between RAS Mutant and RAS Wild Type Cell Lines

The following experiments were conducted as per the protocol given in examples 1-5.

Analysis Groups

The cell lines were divided into two groups based on the RAS mutation status.

Group 1: Cell lines harboring mutant RAS.

Group 2: Cell lines harboring wild type RAS.

FIG. 76 illustrates that cell lines harboring mutant RAS (A549, HCT116, H1299) showed a significant reduction in the percentage of viable cells upon incubation of the cells with both Sorafenib and Atorvastatin. The percentage of viable cells was about 35% in HCT116 cells and about 52% in H1299 cells. The effect on A549 was comparatively less using 5 μM of Sorafenib and 10 μM of Atorvastatin as compared to other RAS mutant cell lines. However, at higher drug doses, A549 cells are found to be significantly affected (as shown in Example 7). In contrast, cell lines with wild type RAS did not exhibit a significant reduction in cell viability. The percentage of viable cells was about 126% in H1650 cells, about 88% in CaCo2 cells, and about 95% in SW48 cells.

### Example 7

### Effect of Sorafenib-Atorvastatin (Sorafenib, 5 μM, and Atorvastatin, 40 μM) Compared Between RAS Mutant and RAS Wild Type Cell Lines

The following experiments were conducted as per the protocol in examples 1-5.

Analysis Groups

The cell lines were divided into two groups based on the RAS mutation status.

Group 1: Cell lines harboring mutant RAS.

Group 2: Cell lines harboring wild type RAS.

FIG. 77 illustrates that cell lines harboring mutant RAS (A549, HCT116, H1299) showed a significant reduction in the percentage of viable cells upon incubation of the cells with both Sorafenib and Atorvastatin. The percentage of viable cells was about 44% in A549, 19% in HCT116 cells and about 37% in H1299 cells. In contrast, cell lines with wild type RAS did not exhibit a significant reduction in cell viability. The percentage of viable cells was about 108% in H1650 cells, about 73% in CaCo2 cells, and about 46% in SW48 cells.

### Example 8

### Effect of Sorafenib-BMS-303141 in the A549 Human Lung Cancer Cell Line

The A549 human lung cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (3,010 cells/well; passage 3). DMSO, digitoxin, and the compounds (Sorafenib and BMS-303141 at a concentration of about 0 μM to about 5 μM and about 0 μM to about 20 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 68 hours followed by the addition of 20 μl of CellTiter 96 Aqueous One Solution Reagent (Promega) to each well. The absorbance of the sample was read at 490 nm.

FIG. 78 illustrates that when the cells were incubated with Sorafenib and BMS-303141 individually, a negligible decrease in A549 cell viability was observed. However, some reduction in viability was observed with a higher dose of BMS-303141, which was enhanced with Sorafenib at 3 μM and 5 μM. Further, FIG. 83 illustrates that the relative growth percentage of the cells with 5 μM Sorafenib was about 89% and with 20 μM BMS-303141, about 105%. Upon incubating the cells with both Sorafenib (5 μM) and BMS-303141 (20 μM), a synergistic effect was observed with a relative growth of about 23%, which is about a 70% reduction in cell viability.

### Example 9

### Effect of Sorafenib-BMS-303141 on the H1650 Human Lung Cancer Cell Line

The H1650 human lung cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (8,150 cells/well; passage 3). DMSO, digitoxin, and the compounds (Sorafenib and BMS-303141 at a concentration of about 0 μM to about 5 μM and about 0 μM to about 20 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 68 hours followed by the addition of 20 μl of CellTiter 96 Aqueous One Solution Reagent (Promega) to each well. The absorbance of the sample was read at 490 nm.

FIG. 79 illustrates that when the cells were incubated with Sorafenib and BMS-303141 individually, a minor decrease in H1650 viability was observed, but was much less than that observed for the A549 cell line. Further, FIG. 83 illustrates that the relative growth percentage of the cells with 5 μM Sorafenib was about 47% and with 20 μM BMS-303141, about 76%. Upon incubating the cells with both Sorafenib (5 μM) and BMS-303141 (20 μM), a synergistic effect was observed with a relative growth of about 29%.

### Example 10

### Effect of Sorafenib-BMS-303141 on the HCT116 Human Colon Cancer Cell Line

The HCT116 human colon cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (3,000 cells/well; passage 3). DMSO, digitoxin, and the compounds (Sorafenib and BMS-303141 at a concentration of about 0 μM to about 5 μM and about 0 μM to about 20 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 80 illustrates that when the cells were incubated with Sorafenib and BMS-303141 individually, a decrease in HCT116 cell viability was observed. There was some reduction in viability with a higher dose of BMS-303141, which was enhanced with Sorafenib at 3 μM and 5 μM. Further, FIG. 83 illustrates that the relative growth percentage of the cells with 5 μM Sorafenib was about 24% and with 20 μM BMS-303141, about 54%. Upon incubating the cells with both Sorafenib (5 μM) and BMS-303141 (20 μM), a synergistic effect was observed with a relative growth of about 1.6%, which is about a 98% reduction in cell viability.

### Example 11

### Effect of Sorafenib-BMS-303141 on the SW48 Human Colon Cancer Cell Line

The SW48 human colon cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (4,980 cells/well; passage 3). DMSO, digitoxin, and the compounds (Sorafenib and BMS-303141 at a concentration of about 0 μM to about 5 μM and about 0 μM to about 20 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 68 hours followed by the addition of 20 μl of CellTiter 96 Aqueous One Solution Reagent (Promega) to each well. The absorbance of the sample was read at 490 nm.

FIG. 81 illustrates that when the cells were incubated with Sorafenib and BMS-303141 individually, a minimal decrease in SW48 cell viability was observed. In combination with higher doses of BMS-303141, a further decrease in cell viability was observed, but this reduction was less that that observed in the RAS mutant cells. FIG. 83 illustrates that the relative growth percentage of the cells with 5 μM Sorafenib was about 80% and with 20 μM BMS-303141, about 78%. Upon incubating the cells with both Sorafenib (5 μM) and BMS-303141 (20 μM), the relative growth was about 35%.

### Example 12

### Effect of Sorafenib-BMS-303141 (Sorafenib, 3 μM, and BMS-303141, 20 μM) Compared Between RAS Mutant and RAS Wild Type Cell Lines

The following experiments were conducted as per the protocol in examples 8-11.

Analysis Groups

The cell lines were divided into two groups based on the RAS mutation status.

Group 1: Cell lines harboring mutant RAS.

Group 2: Cell lines harboring wild type RAS.

FIG. 82 illustrates that cell lines harboring mutant RAS (A549, HCT116) showed a significant reduction in the percentage of viable cells upon incubation of the cells with both Sorafenib and BMS-303141. The percentage of viable cells was about 28% in A549 cells and about 7% in HCT116 cells. In contrast, cell lines with wild type RAS exhibited less of a reduction in cell viability. The percentage of viable cells was about 34% in H1650 cells and about 33% in SW48 cells.

### Example 13

### Effect of Sorafenib-BMS-303141 (Sorafenib, 5 μM, and BMS-303141, 20 μM) Compared Between RAS Mutant and RAS Wild Type Cell Lines

The following experiments were conducted as per the protocol in examples 8-11.

Analysis Groups

The cell lines are divided into two groups based on the RAS mutation status.

Group 1: Cell lines harboring mutant RAS.

Group 2: Cell lines harboring wild type RAS.

Results

FIG. 83 illustrates that cell lines harboring mutant RAS (A549, HCT116) showed a significant reduction in the percentage of viable cells upon incubation of the cells with both Sorafenib and BMS-303141. The percentage of viable cells was about 23% in A549 cells and about 1% in HCT116 cells. In contrast, cell lines with wild type RAS exhibited less of a reduction in cell viability. The percentage of viable cells was about 29% in H1650 cells and about 35% in SW48 cells.

### Example 14

### Effect of ARQ-197-Atorvastatin on the A549 Human Lung Cancer Cell Line

The A549 human lung cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (3,000 cells/well; passage 6). DMSO, digitoxin, and the compounds (ARQ-197 and Atorvastatin at a concentration of about 0 μM to about 3 μM and about 0 μM to about 40 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 84 illustrates that when the A549 cells were incubated with ARQ-197 and Atorvastatin individually or in combination, a decrease in cell viability was observed. The relative growth percentage of the cells with 3 μM ARQ-197 was about 70% and with 40 μM Atorvastatin, about 95%. Further, FIG. 89 illustrates that upon incubating the cells with both ARQ-197 (3 μM) and Atorvastatin (40 μM), a synergistic effect on cell viability was observed with a relative growth of about 54%, which is about a 45% reduction in cell viability.

### Example 15

### Effect of ARQ-197-Atorvastatin on the H1650 Human Lung Cancer Cell Line

The H1650 human lung cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (5,000 cells/well; passage 6). DMSO, digitoxin, and the compounds (ARQ-197 and Atorvastatin at a concentration of about 0 μM to about 3 μM and about 0 μM to about 40 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 85 illustrates that when the H1650 cells were incubated with ARQ-197 and Atorvastatin individually or in combination, a negligible effect on cell viability was observed. The relative growth percentage of the cells with 3 μM ARQ-197 was about 105% and with 40 μM Atorvastatin, about 110%. Further, FIG. 89 illustrates that upon incubating the cells with both ARQ-197 (3 μM) and Atorvastatin (40 μM), a negligible effect on cell viability was observed with a relative growth of about 103%.

### Example 16

### Effect of ARQ-197-Atorvastatin on the HCT116 Human Colon Cancer Cell Line

The HCT116 human colon cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (3,000 cells/well; passage 11). DMSO, digitoxin, and the compounds (ARQ-197 and Atorvastatin at a concentration of about 0 μM to about 3 μM and about 0 μM to about 40 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 86 illustrates that when the HCT116 cells were incubated with ARQ-197 and Atorvastatin individually, a small decrease in cell viability was observed. When both drugs were combined, the decrease in cell viability was enhanced. The relative growth percentage of the cells with 3 μM ARQ-197 was about 63% and with 40 μM Atorvastatin, about 54%. Further, FIG. 89 illustrates that upon incubating the cells with both ARQ-197 (3 μM) and Atorvastatin (40 μM), a synergistic effect on cell viability was observed with a relative growth of about 36%, which is about a 64% reduction in cell viability.

### Example 17

### Effect of ARQ-197-Atorvastatin on the SW48 Human Colon Cancer Cell Line

The SW48 human colon cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (5,300 cells/well; passage 3). DMSO, digitoxin, and the compounds (ARQ-197 and Atorvastatin at a concentration of about 0 μM to about 3 μM and about 0 μM to about 40 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 47 hours followed by the addition of 20 μl of CellTiter 96 Aqueous One Solution Reagent (Promega) to each well. The absorbance of the sample was read at 490 nm.

FIG. 87 illustrates that when the SW48 cells were incubated with ARQ-197 and Atorvastatin individually, a decrease in cell viability was observed that was most pronounced with ARQ-197. When both drugs were combined, a decrease in cell viability was observed. The relative growth percentage of the cells with 3 μM ARQ-197 was about 28% and with 40 μM Atorvastatin, about 66%. Further, FIG. 89 illustrates that upon incubating the cells with both ARQ-197 (3 μM) and Atorvastatin (40 μM) a decrease in cell viability was observed with a relative growth of about 43%. This effect was less than that observed with ARQ-197 alone.

### Example 18

### Effect of ARQ-197-Atorvastatin (ARQ-197, 1 μM, and Atorvastatin, 40 μM) Compared Between RAS Mutant and RAS Wild Type Cell Lines

The following experiments were conducted as per the protocol in examples 14-17.

Analysis Groups

The cell lines were divided into two groups based on RAS mutation status.

Group 1: Cell lines harboring mutant RAS.

Group 2: Cell lines harboring wild type RAS.

FIG. 88 illustrates that cell lines harboring mutant RAS (A549, HCT116) showed a significant reduction in the percentage of viable cells upon incubation of the cells with both ARQ-197 and Atorvastatin as compared to the drugs alone. The percentage of viable cells was about 50% in A549 cells and about 46% in HCT116 cells. Cell lines with wild type RAS exhibited less of a reduction in cell viability when treated with both drugs than with ARQ-197 alone, as observed for the SW48 cells. For the CaCo-2 cells, the reduction in cell viability with both drugs was much less than that seen for the RAS mutant cells. The percentage of viable cells was about 66% in H1650 cells, about 66% in CaCo-2 cells, and about 35% in SW48 cells.

### Example 19

### Effect of ARQ-197-Atorvastatin (ARQ-197, 3 μM, and Atorvastatin, 40 μM) Compared Between RAS Mutant and RAS Wild Type Cell Lines

The following experiments were conducted as per the protocol in examples 14-17.

Analysis Groups

The cell lines were divided into two groups based on RAS mutation status.

Group 1: Cell lines harboring mutant RAS.

Group 2: Cell lines harboring wild type RAS.

FIG. 89 illustrates that cell lines harboring mutant RAS (A549, HCT116) showed a significant reduction in the percentage of viable cells upon incubation of the cells with both ARQ-197 and Atorvastatin as compared to the drugs alone. The percentage of viable cells was about 47% in A549 cells and about 52% in HCT116 cells. Cell lines with wild type RAS exhibited less of a reduction in cell viability. The percentage of viable cells was about 61% in H1650 cells and about 43% in SW48 cells. The significant reduction in cell viability seen in the SW48 cells is due to the effect of ARQ-197 alone.

### Example 20

### Effect of ARQ-197-BMS-303141 on the A549 Human Lung Cancer Cell Line

The A549 human lung cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (3,000 cells/well; passage 6). DMSO, digitoxin, and the compounds (ARQ-197 and BMS-303141 at a concentration of about 0 μM to about 3 μM and about 0 μM to about 20 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 90 illustrates that when the A549 cells were incubated with ARQ-197 or BMS-303141 individually, a minor decrease in cell viability was observed. When both drugs were combined, there was further decrease in cell viability. The relative growth percentage of the cells with 3 μM ARQ-197 was about 71% and with 20 μM BMS-303141, about 105%. Upon incubating the cells with both ARQ-197 (3 μM) and BMS-303141 (20 μM), a synergistic decrease in cell viability was observed with a relative growth of about 53%, which is about a 47% reduction in cell viability.

### Example 21

### Effect of ARQ-197-BMS-303141 on the H1650 Human Lung Cancer Cell Line

The H1650 human lung cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (5,000 cells/well; passage 6). DMSO, digitoxin, and the compounds (ARQ-197 and BMS-303141 at a concentration of about 0 μM to about 3 μM and about 0 μM to about 20 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 91 illustrates that when the H1650 cells were incubated with ARQ-197 or BMS-303141 individually, a minor decrease in cell viability was observed. The relative growth percentage of the cells with 3 μM ARQ-197 was about 52% and with 20 μM BMS-303141, about 76%. Upon incubating the cells with both ARQ-197 (3 μM) and BMS-303141 (20 μM), a considerable decrease in cell viability was observed with a relative growth of about 46%.

### Example 22

### Effect of ARQ-197-Atorvastatin on the HCT116 Human Colon Cancer Cell Line

HCT116 cell line was cultured in McCoy's 5A modified medium containing 10% FBS and 1% Penicillin/Streptomycin. The cells were seeded in 96 well microtitre plates (Nunc) at a density of about 2×10⁴ cells/mL and were allowed to adhere for about 24 hours. The cells were then treated with Atorvastatin and ARQ-197, individually or in combination, wherein the combinations were administered with full titrations of Atorvastatin in the presence of GI₅₀, GI₃₀, and GI₁₅ concentrations of ARQ-197. Upon administration, the cells were incubated for about 72 hours after which the growth medium and compound were removed and replaced with a fixative (1% v/v Trichloroacetic acid (TCA)) and incubated at 4° C. for about 1 hour. Following fixation, the plates were washed with deionized water, Sulforhodamine B (SRB) solution was added, and the cells were incubated for about 30 mins, allowing the staining to occur. Incubation was followed by measuring the optical density at 594 nm (Packard Fusion plate reader) or by measuring fluorescence intensity at Em 590 nm (BMG Pherastar Plus plate reader).

FIG. 92 illustrates the dose response plot of increasing concentrations of Atorvastatin (0.001 μM-100 μM) alone (i.e., with DMSO) or in combination with ARQ-197 at a concentration of GI₁₅, GI₃₀, or GI₅₀ in HCT116 cells. ARQ-197 increased the effectiveness of Atorvastatin. FIG. 93 depicts the zoomed in plot of FIG. 92 between 1-31.6 μM concentrations of Atorvastatin.

FIG. 94 illustrates that 10 μM of Atorvastatin alone showed no decrease in relative growth, while the GI₅₀ concentration of ARQ-197 alone showed about a 23% decrease in relative growth. Additionally, the combination of Atorvastatin (10 μM) and ARQ-197 (GI₅₀) showed about a 50% decrease in relative growth in HCT116 cells.

### Example 23

### Effect of ARQ-197-Atorvastatin on the CAPAN1 Human Pancreatic Cancer Cell Line

CAPAN1 cell line was cultured in Iscove's Modified Dulbecco's Medium (IMDM) with 10% FBS and 1% Penicillin/Streptomycin. The cells were seeded in 96 well microtitre plates (Nunc) at a density of about 2×10⁴ cells/mL, and were allowed to adhere for about 24 hours. The cells were then treated with Atorvastatin and ARQ-197, individually or in combination, wherein the combinations were administered with full titrations of Atorvastatin in the presence of GI₅₀, GI₃₀, and GI₁₅ concentrations of ARQ-197. Upon administration, the cells were incubated for about 72 hours after which the growth medium and compound were removed and replaced with a fixative (1% v/v Trichloroacetic acid (TCA)) and incubated at 4° C. for about 1 hour. Following fixation, the plates were washed with deionized water, Sulforhodamine B (SRB) solution was added, and the cells were incubated for about 30 mins, allowing the staining to occur. Incubation was followed by measuring the optical density at 594 nm (Packard Fusion plate reader) or by measuring fluorescence intensity at Em 590 nm (BMG Pherastar Plus plate reader).

FIG. 95 illustrates the dose response plot of increasing concentrations of Atorvastatin (0.001 μM-100 μM) alone (i.e., with DMSO) or in combination with ARQ-197 at a concentration of GI₁₅, GI₃₀, or GI₅₀ in CAPAN1 cells. ARQ-197 increased the effectiveness of Atorvastatin. FIG. 96 depicts the zoomed in plot of FIG. 95 between 1-31.6 μM concentrations of Atorvastatin.

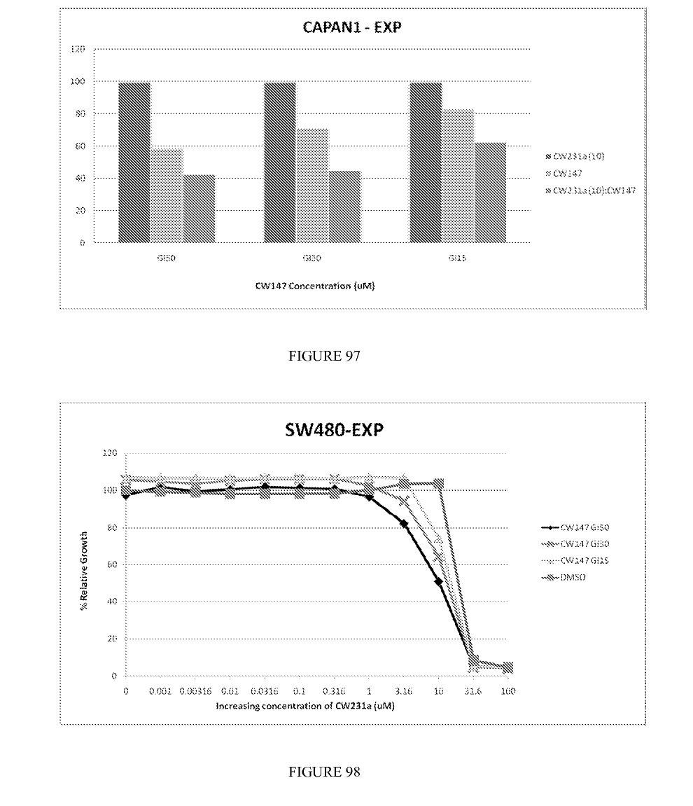

FIG. 97 illustrates that 10 μM of Atorvastatin alone showed no decrease in relative growth, while the GI₅₀ concentration of ARQ-197 alone showed about a 40% decrease in relative growth. Additionally, the combination of Atorvastatin (10 μM) and ARQ-197 (GI₅₀) showed about a 60% decrease in relative growth in HCT116 cells.

### Example 24

### Effect of ARQ-197-Atorvastatin on the SW480 Human Colon Cancer Cell Line

The SW480 cell line was cultured in Dulbecco's Modified Eagle Medium (DMEM) with 10% FBS and 1% Penicillin/Streptomycin. The cells were seeded in 96 well microtitre plates (Nunc) at a density of about 8×10⁴ cells/mL, and were allowed to adhere for about 24 hours. The cells were then treated with Atorvastatin and ARQ-197, individually or in combination, wherein the combinations were administered with full titrations of Atorvastatin in the presence of GI₅₀, GI₃₀, and GI₁₅ concentrations of ARQ-197. Upon administration, the cells were incubated for about 72 hours, after which the growth medium and compound were removed and replaced with a fixative (1% v/v Trichloroacetic acid (TCA)) and incubated at 4° C. for about 1 hour. Following fixation, the plates were washed with deionized water, Sulforhodamine B (SRB) solution was added, and the cells were incubated for about 30 mins, allowing the staining to occur. Incubation was followed by measuring the optical density at 594 nm (Packard Fusion plate reader) or by measuring fluorescence intensity at Em 590 nm (BMG Pherastar Plus plate reader).

FIG. 98 illustrates the dose response plot of increasing concentrations of Atorvastatin (0.001 μM-100 μM) alone (i.e., with DMSO) or in combination with ARQ-197 at a concentration of GI₁₅, GI₃₀, or GI₅₀ in SW480 cells. ARQ-197 increased the effectiveness of Atorvastatin. FIG. 99 depicts the zoomed in plot of FIG. 98 between 1-31.6 μM concentrations of Atorvastatin.

FIG. 100 illustrates that 10 μM of Atorvastatin alone and the GI₅₀ concentration of ARQ-197 alone showed no decrease in relative growth. In contrast, the combination of Atorvastatin (10 μM) and ARQ-197 (GI₅₀) showed about a 50% decrease in relative growth in SW480 cells.

### Example 25

### Effect of ARQ-197-Atorvastatin on the DLD1 Human Colon Cancer Cell Line

The DLD1 cell line was cultured in RPMI 1640 Medium with 10% FBS and 1% Penicillin/Streptomycin. The cells were seeded in 96 well microtitre plates (Nunc) at a density of about 2×10⁴ cells/mL and were allowed to adhere for about 24 hours. The cells were then treated with Atorvastatin and ARQ-197, individually or in combination, wherein the combinations were administered with full titrations of Atorvastatin in the presence of GI₅₀, GI₃₀, and GI₁₅ concentrations of ARQ-197. Upon administration, the cells were incubated for about 72 hours, after which the growth medium and compound were removed and replaced with a fixative (1% v/v Trichloroacetic acid (TCA)) and incubated at 4° C. for about 1 hour. Following fixation, the plates were washed with deionized water, Sulforhodamine B (SRB) solution was added, and the cells were incubated for about 30 mins, allowing the staining to occur. Incubation was followed by measuring the optical density at 594 nm (Packard Fusion plate reader) or by measuring fluorescence intensity at Em 590 nm (BMG Pherastar Plus plate reader).

FIG. 101 illustrates the dose response plot of increasing concentrations of Atorvastatin (0.001 μM-100 μM) alone (i.e., with DMSO) or in combination with ARQ-197 at a concentration of GI₁₅, GI₃₀, or GI₅₀ in SW480 cells. ARQ-197 increased the effectiveness of Atorvastatin. FIG. 102 depicts the zoomed in plot of FIG. 101 between 1-31.6 μM concentrations of Atorvastatin.

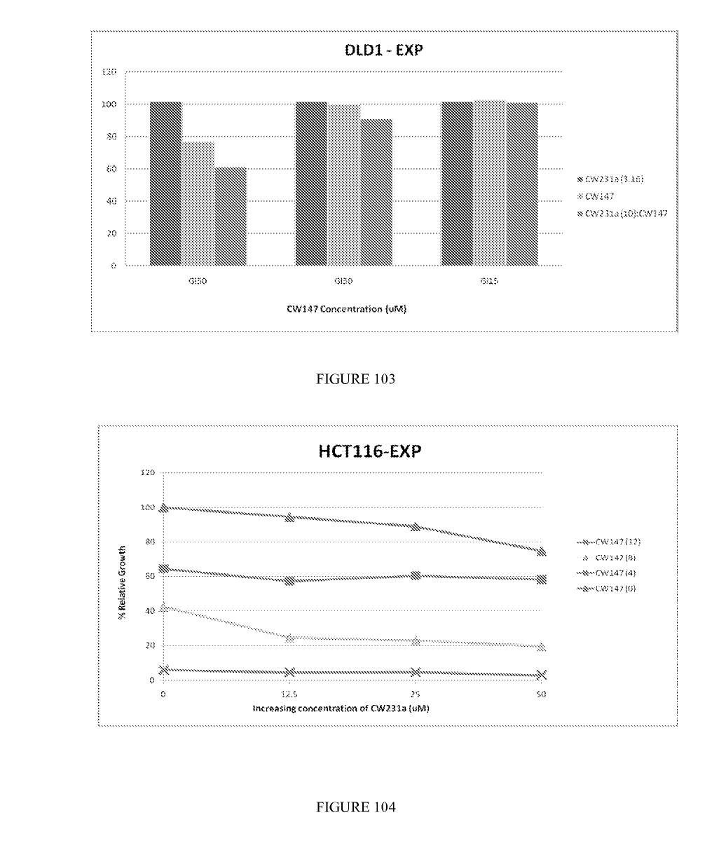

FIG. 103 illustrates that 10 μM of Atorvastatin alone showed no decrease in relative growth, while the GI₅₀ concentration of ARQ-197 alone showed about a 20% decrease in relative growth. Additionally, the combination of Atorvastatin (10 μM) and ARQ-197 (GI₅₀) showed about a 40% decrease in relative growth in DLD1 cells.

### Example 26

### Effect of Crizotinib-Atorvastatin on the HCT116 Human Colon Cancer Cell Line

The HCT116 human colon cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (3,000 cells/well; passage 3). DMSO, digitoxin, and the compounds (Crizotinib and Atorvastatin at a concentration of about 0 μM, 4 μM, 8 μM, and 12 μM, and about 0 μM, 12.5 μM, 25 μM, and 50 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 104 illustrates the dose response plot of increasing concentrations of Atorvastatin (0 μM-50 μM) alone or in combination with Crizotinib at a concentration of 4 μM, 8 μM, or 12 μM in HCT116 cells. Crizotinib increased the effectiveness of Atorvastatin.

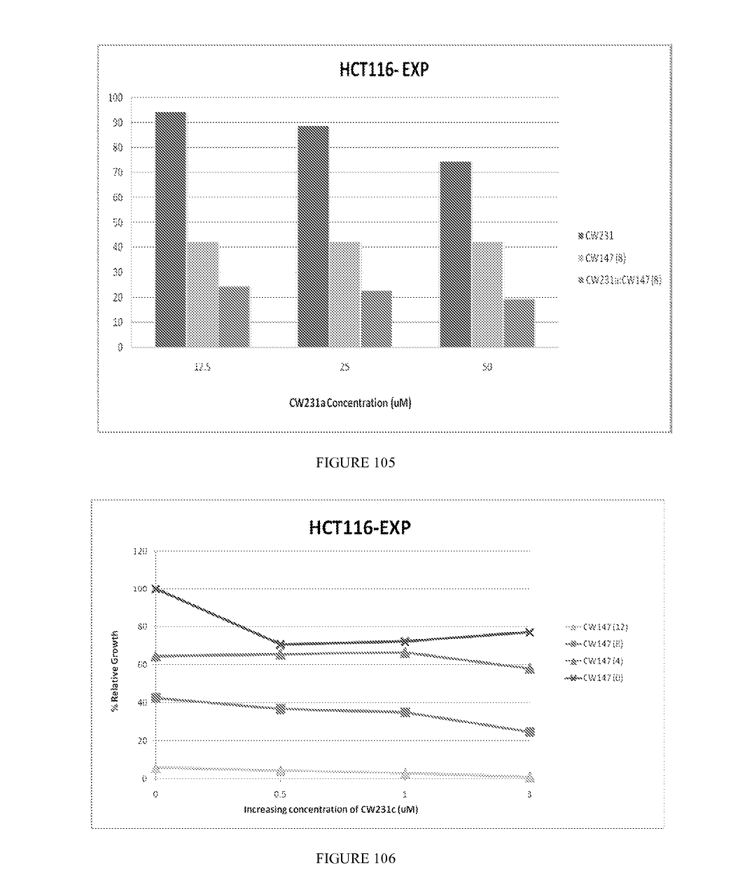

FIG. 105 illustrates that 12.5 μM of Atorvastatin alone showed less than 10% decrease in relative growth, while 8 μM of Crizotinib alone showed about a 60% decrease in relative growth (FIG. 104). Additionally, the combination of Atorvastatin (12.5 μM) and Crizotinib (8 μM) showed greater than a 75% decrease in relative growth in the HCT116 cell line.

### Example 27

### Effect of Crizotinib-Tipifamib on the HCT116 Human Colon Cancer Cell Line

The HCT116 human colon cancer cell line was procured from the ATCC (American Type Culture Collection, Manassas, Va.). The cells were resuspended in media containing 10% FBS (Gibco) and 4× Gentamicin followed by transferring about 100 μl of the resuspension to each well in an assay plate (3,000 cells/well; passage 3). DMSO, digitoxin, and the compounds (Crizotinib and Tipifamib at a concentration of about 0 μM, 4 μM, 8 μM, and 12 μM, and about 0 μM, 0.5 μM, 1 μM, and 3 μM, respectively) were serially diluted in assay media. 100 μl of the diluted sample was added to each well of the assay plate containing the resuspended cells. The final assay volume of each well was about 200 μl containing 10% FBS, 2× Gentamicin, DMSO, digitoxin, and drugs. The assay plate was incubated at 37° C. for about 66 hours followed by the addition of 20 μl of CellTiter 96 Aqueous One Solution Reagent (Promega) to each well. The absorbance of the sample was read at 490 nm.

FIG. 106 illustrates the dose response plot of increasing concentrations of Tipifamib (0 μM-3 μM) alone or in combination with Crizotinib at fixed doses of 4 μM, 8 μM, or 12 μM in HCT116 cells. Crizotinib increased the effectiveness of Tipifamib.

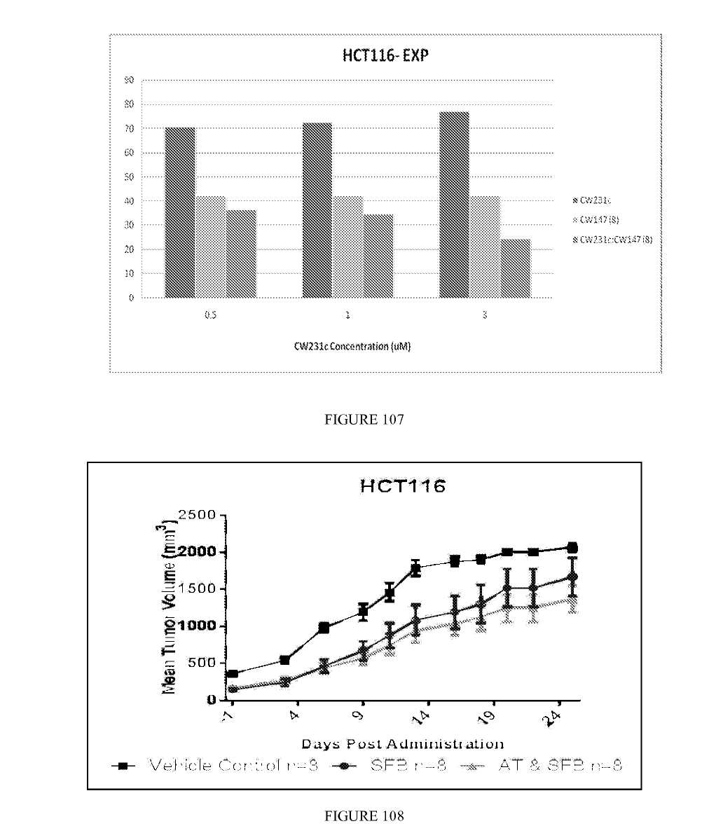

FIG. 107 illustrates that 3 μM of Tipifamib alone showed about a 22% decrease in relative growth, while 8 μM of Crizotinib alone showed about a 58% decrease in relative growth (FIG. 104). Additionally, the combination of Tipifamib (3 μM) and Crizotinib (8 μM) showed about a 75% decrease in relative growth in the HCT116 cell line.

### Example 28

### Effect of Sorafenib-Atorvastatin on the Mouse Xenograft Model of HCT116

The following experiment was carried out in immunocompromised mice (Harlan Athymic Nude) between 4-6 weeks of age. The HCT116 cell line used for this experiment is identical to the cell line used for previous in vitro analyses.

The mouse was injected unilaterally on the right flank with 1 million tumor cells (HCT116) and mixed with an equal volume of BD matrigel. Pre-study tumor volumes were recorded for each experiment one week prior to the start date and every third day thereafter through the duration of the experiment. Administration of the Sorafenib and Atorvastatina, individually or in combination, was started when the tumor volume was about 100-150 mm³.

FIG. 108 illustrates the effect of Sorafenib and Atorvastatin alone and in combination on the HCT116 mouse xenograft model. The inhibition of tumor volume by Sorafenib alone was 16% and the combination of Sorafenib and Atorvastatin was able to inhibit tumor growth by about 34% at 19 days. A significant difference was seen among the combination-treated cohort, the untreated cohort, and the cohort treated with only Sorafenib. This result indicates that the combination treatment can be more effective in reducing tumor volume.

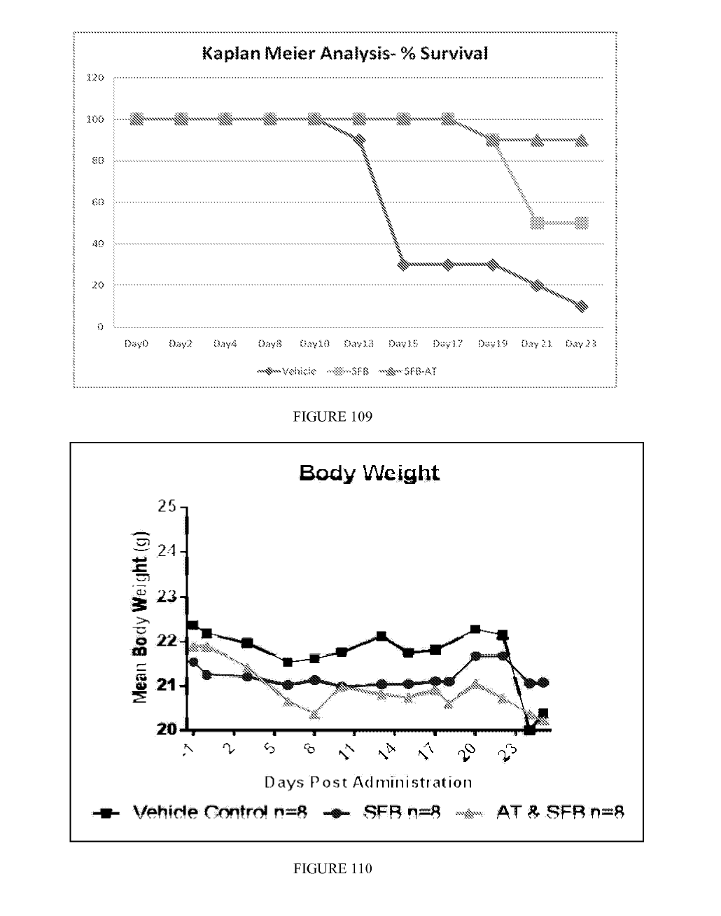

FIG. 109 illustrates the survival curve for the xenografted mice from FIG. 108. The curve indicates that mice treated with either Sorafenib, alone or in combination with Atorvastatin, had prolonged survival rates.

FIG. 110 illustrates the body weight of the xenografted mice from FIG. 108. The plot indicates that the body weight of the mice was not significantly affected by treatment with either Sorafenib or the combination of Sorafenib and Atorvastatin. This result further implies lack of toxicity during the duration of the study.

### Example 29

### Effect of Sorafenib-Atorvastatin on a Mouse Xenograft Model of CAPAN1

The following experiment was carried out in immunocompromised mice (Harlan Athymic Nude) between 4-6 weeks of age. The CAPAN1 cell line used for this experiment was identical to the cell line used for previous in vitro analyses.

The mouse was injected unilaterally on the right flank with 1 million tumor cells (CAPAN1) and mixed with an equal volume of BD matrigel. Pre-study tumor volumes were recorded for each experiment one week prior to the start date and every third day thereafter through the duration of the experiment. Administration of the Sorafenib and Atorvastatin, individually or in combination, was started when the tumor volume was about 100-150 mm³.

FIG. 111 illustrates the effect of Sorafenib and Atorvastatin alone and in combination on the CAPAN1 mouse xenograft model. The combination of Sorafenib and Atorvastatin inhibited tumor growth by about 42% at 19 days. A significant difference was seen between the combination-treated cohort and the untreated cohort, indicating that the combination treatment can be effective in reducing tumor volume.

FIG. 112 illustrates the survival curve for the xenografted mice from FIG. 111. The curve indicates that mice treated with either Sorafenib, alone or in combination with Atorvastatin, had slightly prolonged survival rates.

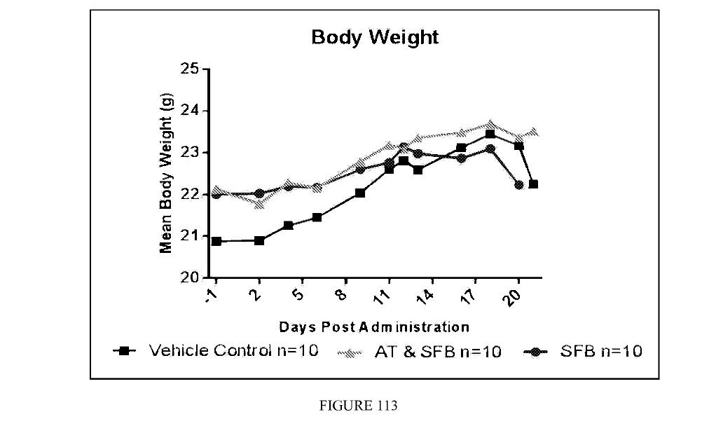

FIG. 113 illustrates the body weight of the xenografted mice from FIG. 111. The plot indicates that the body weight of the mice was not significantly affected by treatment with either Sorafenib or the combination of Sorafenib and Atorvastatin. This result further implies a lack of toxicity during the duration of the study.

In the examples illustrated above, the concentration of drugs (CW137, CW147, CW231a, CW231b and CW231c) was gradually increased individually or in combination during the assay to analyse the effect of different drug dosages on the cell line. A similar protocol was followed for different two drug combinations of the compounds during the assay stage to analyse the effect of the combination of drugs at different dosage on the KRAS mutant and wild type cell lines, respectively.

1-132. (canceled)

133. A pharmaceutical composition comprising:
a) i) c-MET inhibitor or RAF inhibitor or a pharmaceutically-acceptable salt of any of the foregoing; and
ii) prenylation inhibitor or a pharmaceutically-acceptable salt thereof; and
b) a pharmaceutically-acceptable excipient,
wherein the composition is a unit dosage form, and exerts a substantially enhanced effect on treatment of cancer having RAS mutation, when compared to treatment with either (i) or (ii) individually.

134. The pharmaceutical composition of claim 133, wherein the prenylation inhibitor is selected from a group comprising HMG-CoA reductase inhibitor, ATP citrate lyase inhibitor, farnesyl diphosphate synthase inhibitor, farnesyl transferase inhibitor, and geranylgeranyl transferase inhibitor;
wherein the c-MET inhibitor is selected from a group comprising crizotinib, ARQ-197, PF-04217903, JNJ38877605, PHA-665752, SU11274, INCB28060, AMG-208, NVP-BVU972, BMS-777607, SGX-523 and pharmaceutically-acceptable salt of any of the foregoing, or any combination thereof; and
wherein the RAF inhibitor is selected from a group comprising vemurafenib, regorafenib, sorafenib, GDC-0879, PLX-4720, RAF265, NVP-BHG712, SB590885, ZM 336372, AZ628 and a pharmaceutically-acceptable salt of any of the foregoing, or any combination thereof.

135. The pharmaceutical composition of claim 133, wherein the prenylation inhibitor is HMG-CoA reductase inhibitor selected from a group comprising simvastatin, atorvastatin, rosuvastatin, cerivastatin, fluvastatin, lovastatin, mevastatin, pitavastatin, pravastatin and a pharmaceutically-acceptable salt of any of the foregoing, or any combination thereof; or
wherein the prenylation inhibitor is ATP citrate lyase inhibitor selected from a group comprising BMS-303141, SB 204990, SB 201076, ETC-I002 and a pharmaceutically-acceptable salt of any of the foregoing, or any combination thereof; or
wherein the prenylation inhibitor is farnesyl diphosphate synthase inhibitor selected from a group comprising risedronate, zoledronate, ibandronate, alendronate, pamidronate, etidronate, clodronate, neridronate, tiludronate, incadronate, olpadronate, EB-1053, minodronate, and a pharmaceutically-acceptable salt of any of the foregoing, or any combination thereof; or
wherein the prenylation inhibitor is farnesyl transferase inhibitor selected from a group comprising tipifarnib, lonafarnib, BMS-214662, L778123, L-731734, B1086, L-744832, BIM-46228, FTI-276, RPR-130401, FTI-2148, FTI-2628, FTI-277, FTase Inhibitor I, FTase Inhibitor II, FTase Inhibitor III, L-745631, L-739749 and a pharmaceutically-acceptable salt of any of the foregoing, or any combination thereof; or
wherein the prenylation inhibitor is geranylgeranyl transferase inhibitor selected from a group comprising GGTI-298, GGTI-2418, GGTI-2133, GGTI-2147, GGTI-2154, GGTI-2166, GGTI-286, GGTI-287, GGTI-297 and a pharmaceutically-acceptable salt of any of the foregoing, or any combination thereof.

136. The pharmaceutical composition of claim 133, wherein the unit dosage form is formulated for oral administration for cancer having RAS mutation.

137. The pharmaceutical composition of claim 133, wherein each of the c-MET inhibitor or the RAF inhibitor or the pharmaceutically-acceptable salt thereof and the prenylation inhibitor or the pharmaceutically-acceptable salt thereof is present in an amount from about 10% to about 50% of a maximum tolerated dose or in an amount from about 1 mg to about 2000 mg for cancer having RAS mutation.

138. The pharmaceutical composition of claim 133, wherein the cancer having RAS mutation is selected from a group comprising pancreatic cancer, colorectal cancer, lung cancer, breast cancer, brain cancer, head and neck cancer, multiple myeloma, acute non-lymphocytic leukemia and myelodysplasia, or any combination thereof.

139. A kit for cancer having RAS mutation comprising:
a) i) c-MET inhibitor or RAF inhibitor or a pharmaceutically-acceptable salt thereof; and
ii) prenylation inhibitor or a pharmaceutically-acceptable salt thereof; and
b) written instructions on use of the kit for treatment of a condition.

140. The kit of claim 139, wherein the written instructions describe use of the kit for treatment of a cancer having RAS mutation.

141. The kit of claim 139, wherein the kit comprises a unit dosage form; and
wherein the unit dosage form contains the c-MET inhibitor or RAF inhibitor or the pharmaceutically-acceptable salt thereof and the prenylation inhibitor or the pharmaceutically-acceptable salt thereof for cancer having RAS mutation; or
wherein the unit dosage form contains the c-MET inhibitor or the RAF inhibitor or the pharmaceutically-acceptable salt thereof; and wherein the kit further comprises a second unit dosage form containing the prenylation inhibitor or the pharmaceutically-acceptable salt thereof for cancer having RAS mutation.

142. The kit of claim 139, wherein the prenylation inhibitor is selected from a group comprising HMG-CoA reductase inhibitor, ATP citrate lyase inhibitor, farnesyl diphosphate synthase inhibitor, farnesyl transferase inhibitor, and geranylgeranyl transferase inhibitor;
wherein the c-MET inhibitor is selected from a group comprising crizotinib, ARQ-197, PF-04217903, JNJ38877605, PHA-665752, SU11274, INCB28060, AMG-208, NVP-BVU972, BMS-777607, SGX-523 and pharmaceutically-acceptable salt of any of the foregoing, or any combination thereof; and
wherein the RAF inhibitor is selected from a group comprising vemurafenib, regorafenib, sorafenib, GDC-0879, PLX-4720, RAF265, NVP-BHG712, SB590885, ZM 336372, AZ628 and a pharmaceutically-acceptable salt of any of the foregoing, or any combination thereof.

143. The kit of claim 139, wherein the prenylation inhibitor is HMG-CoA reductase inhibitor selected from a group comprising simvastatin, atorvastatin and rosuvastatin, cerivastatin, fluvastatin, lovastatin, mevastatin, pitavastatin, pravastatin and a pharmaceutically-acceptable salt of any of the foregoing, or any combination thereof; or
wherein the prenylation inhibitor is ATP citrate lyase inhibitor selected from a group comprising BMS-303141, SB 204990, SB 201076, ETC-I002 and a pharmaceutically-acceptable salt of any of the foregoing, or any combination thereof; or
wherein the prenylation inhibitor is farnesyl diphosphate synthase inhibitor selected from a group comprising risedronate, zoledronate, ibandronate, alendronate, pamidronate, etidronate, clodronate, neridronate, tiludronate, incadronate, olpadronate, EB-1053, minodronate, and a pharmaceutically-acceptable salt of any of the foregoing, or any combination thereof; or
wherein the prenylation inhibitor is farnesyl transferase inhibitor selected from a group comprising tipifarnib, lonafarnib, BMS-214662, L778123, L-731734, B1086, L-744832, BIM-46228, FTI-276, RPR-130401, FTI-2148, FTI-2628, FTI-277, FTase Inhibitor I, FTase Inhibitor II, FTase Inhibitor III, L-745631, L-739749 and a pharmaceutically-acceptable salt of any of the foregoing, or any combination thereof; or
wherein the prenylation inhibitor is geranylgeranyl transferase inhibitor selected from a group comprising GGTI-298, GGTI-2418, GGTI-2133, GGTI-2147, GGTI-2154, GGTI-2166, GGTI-286, GGTI-287, GGTI- and a pharmaceutically-acceptable salt of any of the foregoing, or any combination thereof.

144. The kit of claim 139, wherein the c-MET inhibitor or RAF inhibitor or the pharmaceutically-acceptable salt thereof; and the prenylation inhibitor or the pharmaceutically-acceptable salt thereof are administered sequentially for treating cancer having RAS mutation; or
wherein the c-MET inhibitor or RAF inhibitor or the pharmaceutically-acceptable salt thereof; and the prenylation inhibitor or the pharmaceutically-acceptable salt thereof are administered simultaneously for treating cancer having RAS mutation.

145. The kit of claim 139, wherein the c-MET inhibitor or RAF inhibitor or the pharmaceutically-acceptable salt thereof is from about 10% to about 50% of a maximum tolerated dose, and the prenylation inhibitor or the pharmaceutically-acceptable salt thereof is from about 10% to about 50% of a maximum tolerated dose for treating cancer having RAS mutation; or
wherein the c-MET inhibitor or RAF inhibitor or the pharmaceutically-acceptable salt thereof is from about 1 mg to about 2000 mg, and the prenylation inhibitor or the pharmaceutically-acceptable salt thereof is from about 1 mg to about 2000 mg for treating cancer having RAS mutation.

146. The kit of claim 139, wherein the cancer having RAS mutation is selected from a group comprising pancreatic cancer, colorectal cancer, lung cancer, breast cancer, brain cancer, head and neck cancer, multiple myeloma, acute non-lymphocytic leukemia and myelodysplasia, or any combination thereof.

147. A method for treating cancer having RAS mutation in a subject in need thereof, the method comprising administering to the subject a composition comprising:
i) a therapeutically-effective amount of c-MET inhibitor or RAF inhibitor or a pharmaceutically-acceptable salt thereof; and
ii) a therapeutically-effective amount of prenylation inhibitor or a pharmaceutically-acceptable salt thereof;
wherein the composition exerts a substantially enhanced effect on treatment of cancer having RAS mutation, when compared to treatment with either (i) or (ii) individually.

148. The method of claim 147, wherein the prenylation inhibitor is selected from a group comprising HMG-CoA reductase inhibitor, ATP citrate lyase inhibitor, farnesyl diphosphate synthase inhibitor, farnesyl transferase inhibitor, and geranylgeranyl transferase inhibitor;
wherein the c-MET inhibitor is selected from a group comprising crizotinib, ARQ-197, PF-04217903, JNJ38877605, PHA-665752, SU11274, INCB28060, AMG-208, NVP-BVU972, BMS-777607, SGX-523 and pharmaceutically-acceptable salt of any of the foregoing, or any combination thereof; and
wherein the RAF inhibitor is selected from a group comprising vemurafenib, regorafenib, sorafenib, GDC-0879, PLX-4720, RAF265, NVP-BHG712, SB590885, ZM 336372, AZ628 and a pharmaceutically-acceptable salt of any of the foregoing, or any combination thereof.

149. The method of claim 147, wherein the prenylation inhibitor is HMG-CoA reductase inhibitor selected from a group comprising simvastatin, atorvastatin and rosuvastatin, cerivastatin, fluvastatin, lovastatin, mevastatin, pitavastatin, pravastatin and a pharmaceutically-acceptable salt of any of the foregoing, or any combination thereof; or
wherein the prenylation inhibitor is ATP citrate lyase inhibitor selected from a group comprising BMS-303141, SB 204990, SB 201076, ETC-I002 and a pharmaceutically-acceptable salt of any of the foregoing, or any combination thereof; or
wherein the prenylation inhibitor is farnesyl diphosphate synthase inhibitor selected from a group comprising risedronate, zoledronate, ibandronate, alendronate, pamidronate, etidronate, clodronate, neridronate, tiludronate, incadronate, olpadronate, EB-1053, minodronate, and a pharmaceutically-acceptable salt of any of the foregoing, or any combination thereof; or
wherein the prenylation inhibitor is farnesyl transferase inhibitor selected from a group comprising tipifarnib, lonafarnib, BMS-214662, L778123, L-731734, B1086, L-744832, BIM-46228, FTI-276, RPR-130401, FTI-2148, FTI-2628, FTI-277, FTase Inhibitor I, FTase Inhibitor II, FTase Inhibitor III, L-745631, L-739749 and a pharmaceutically-acceptable salt of any of the foregoing, or any combination thereof; or
wherein the prenylation inhibitor is geranylgeranyl transferase inhibitor selected from a group comprising GGTI-298, GGTI-2418, GGTI-2133, GGTI-2147, GGTI-2154, GGTI-2166, GGTI-286, GGTI-287, GGTI- and a pharmaceutically-acceptable salt of any of the foregoing, or any combination thereof.

150. The method of claim 147, wherein the c-MET inhibitor or RAF inhibitor or the pharmaceutically-acceptable salt thereof; and the prenylation inhibitor or the pharmaceutically-acceptable salt thereof are administered sequentially for treating cancer having RAS mutation; or
wherein the c-MET inhibitor or RAF inhibitor or the pharmaceutically-acceptable salt thereof; and the prenylation inhibitor or the pharmaceutically-acceptable salt thereof are administered simultaneously for treating cancer having RAS mutation.

151. The method of claim 147, wherein the c-MET inhibitor or the RAF inhibitor or the pharmaceutically-acceptable salt thereof; and the prenylation inhibitor or the pharmaceutically-acceptable salt thereof are administered in a unit dosage form for treating cancer having RAS mutation.

152. The method of claim 147, wherein the c-MET inhibitor or RAF inhibitor or the pharmaceutically-acceptable salt thereof is from about 10% to about 50% of a maximum tolerated dose, and the prenylation inhibitor or the pharmaceutically-acceptable salt thereof is from about 10% to about 50% of a maximum tolerated dose for treating cancer having RAS mutation; or
wherein the c-MET inhibitor or RAF inhibitor or the pharmaceutically-acceptable salt thereof is from about 1 mg to about 2000 mg, and the prenylation inhibitor or the pharmaceutically-acceptable salt thereof is from about 1 mg to about 2000 mg for treating cancer having RAS mutation.

153. The method of claim 147, wherein the cancer having RAS mutation is selected from a group comprising pancreatic cancer, colorectal cancer, lung cancer, breast cancer, brain cancer, head and neck cancer, multiple myeloma, acute non-lymphocytic leukemia and myelodysplasia, or any combination thereof.

154. A process for preparing the composition of claim 133, said process comprising acts of combining the c-MET inhibitor or the RAF inhibitor or the pharmaceutically-acceptable salt thereof and the prenylation inhibitor or the pharmaceutically-acceptable salt thereof, along with the pharmaceutically-acceptable excipient for treating cancer having RAS mutation.
